(19) United States
(12) Patent Application Publication
(10) Pub. No.: US 2026/0014240 A1
(43) Pub. Date: Jan. 15, 2026

(54) RATIONALLY DESIGNED MYCOPLASMA GALLISEPTICUM SUBUNIT VACCINE

(71) Applicant: University of Connecticut, Farmington, CT (US)

(72) Inventors: Steven J. Geary, Storrs, CT (US); Edan R. Tulman, Storrs, CT (US); Steven M. Szczepanek, Coventry, CT (US)

(21) Appl. No.: 18/994,954

(22) PCT Filed: Jul. 20, 2023
(86) PCT No.: PCT/US2023/070600

§ 371 Date: Jan. 15, 2025

Related U.S. Application Data
(60) Provisional application No. 63/390,756, filed on Jul. 20, 2022.

(51) Int. Cl. A61K 39/02; A61K 39/00

(52) U.S. Cl. CPC A61K 39/0241; A61K 2039/54; A61K 2039/545; A61K 2039/552; A61K 2039/55566

(57) ABSTRACT

A novel recombinant subunit vaccine composition against avian Mycoplasma infection is described. The vaccine includes VlhA early phase antigens chosen based on their expression profile during the first 7 days of infection. The vaccine composition is shown to induce a protective immune response in chickens. Vaccine compositions and methods of making and using the vaccine compositions are described.

### CROSS-REFERENCE TO RELATED APPLICATION

This application claims the benefit of U.S. Provisional Application No. 63/390,756 filed on Jul. 20, 2022, which is incorporated herein by reference in its entirety.

### FEDERAL RESEARCH STATEMENT

This invention was made with government support under grant numbers R01 AI17321-01 and AI057552 awarded by the National Institutes of Health. The government has certain rights in the invention.

### INCORPORATION-BY-REFERENCE OF MATERIAL ELECTRONICALLY SUBMITTED

Incorporated by reference in its entirety is a computer readable nucleotide/amino acid sequence listing submitted concurrently herewith as an 176,128 bytes XML file named “UCT0292PCT Sequence Listing Jul. 20, 2023.XML”, created on Jul. 20, 2023.

### BACKGROUND

Mycoplasma gallisepticum (MG) is the primary etiologic agent of avian mycoplasmosis, a respiratory disease of significance and which places infected poultry at a significantly increased risk of developing co-infections with other pathogens. This can lead to a pathological condition known as chronic respiratory disease (CRD), which substantially compromises the integrity and function of the respiratory tract. Significant economic losses in poultry from chronic MG infection occur due to reduced egg production and hatchability, as well as downgrading of carcasses.

MG is readily transmitted via inhalation of aerosolized respiratory secretions. Currently, MG control strategies include strict biosecurity to maintain MG-free breeder flocks and vaccination programs on large-scale poultry farms. The two types of commercially available vaccines for MG include bacterins, which are an inactivated suspension of whole bacteria, and live-attenuated vaccines (LAV). Current bacterins do not protect well against infection, especially when birds are exposed to heterologous strains. However, bacterin use in commercial pullets can reduce declines in egg production associated with MG infection. MG-Bac® (manufactured by Zoetis) provides limited protection against field strains but doesn't reduce horizontal transmission between laying hens. It also provides minimal long-term MG control in multiple-age commercial layers. This is likely due to the “snapshot” of antigens presented by MG at the time when it is inactivated in the vaccine formulation process. There is a great deal of room for improvement over MG-Bac®. LAVs express variable antigens throughout their persistence in the host, providing the opportunity for exposure to a broader range of the protective antigens, resulting in greater protection. Despite this benefit the issue of safety remains. Unfortunately, the literature is full of reports of LAVs reverting to virulence.

There are three commercially available LAVs: F strain, Vaxsafe MG (ts-11), and 6/85. The F strain vaccine (derived from a naturally attenuated strain) elicits a strong serological response, is reported to provide good protection for layers has been shown to displace the pathogenic R strain from infected flocks and can be readily distinguished from field strains. However, F strain is pathogenic for turkeys and young chickens and has been shown to mildly impair the reproductive performance of layers. The vaccine strains ts-11 (a temperature sensitive mutant) and 6/85 (generated by serial passage in-vitro) do not afford the same level of protection as the F strain vaccine, but they are safer in commercial poultry, particularly turkeys. In addition, ts-11 and 6/85 have no discernable impact on egg production.

A modified Vaxsafe® MG (strain ts-304) vaccine has recently been reported. In contrast to the original Vaxsafe® MG (ts-11), it possesses the primary cytadhesin molecule GapA, which has been shown to be essential for its increased efficacy. Although the 6/85 vaccine is difficult to recover after vaccination, 6/85-like isolates have been recovered from unvaccinated birds, raising the possibility of reversion to virulence. The molecular nature of the attenuating mutations in the existing MG vaccines are not defined, and despite the fact that these MG vaccines are available, still, none to date provide adequate protection for the effective control of MG. Given these shortcomings (inadequate protection, residual virulence, and the potential for reversion to wild-type virulence), the poultry industry continues to lack a suitable, safe, and effective vaccine to prevent avian respiratory mycoplasmosis.

### BRIEF SUMMARY

In an aspect, a Mycoplasma gallisepticum (MG) subunit vaccine comprises recombinant gallisepticum adhesion protein A (GapA) antigen, recombinant cytadhesin-related molecule A (CrmA) protein antigens, and one or more recombinantly produced early phase variable lipoprotein and hemagglutinin (VlhA) antigens; and an immune-enhancing amount of an adjuvant. In one aspect, VlhA antigens comprise VlhA 3.03, VlhA 3.06, VlhA 4.07, VlhA 4.08 and VlhA 5.05 antigens. In yet another aspect, a method of immunizing an avian against infection with Mycoplasma gallisepticum comprising administering to the avian the subunit vaccine in an amount sufficient to induce a protective immune response.

In another aspect, a method of making a Mycoplasma gallisepticum (MG) subunit vaccine composition comprises determining an early phase variable lipoprotein hemagglutinin (VlhA) antigen expression profile from MG infected birds, selecting a first early phase VhlA antigen with a maximum expression at a first time post-infection, selecting a second early phase VlhA antigen with a maximum expression at a second time post-infection, recombinantly expressing the first and second early phase VlhA antigens, recombinantly expressing the primary MG adhesion protein A (GapA) antigen, recombinantly expressing the cytadherence related protein MG molecule A (CrmA) protein antigen, and combining the recombinantly expressed first and second early phase VhlA antigens, the GapA antigen, the CrmA antigen, and an immune enhancing amount of an adjuvant to provide the vaccine.

### BRIEF DESCRIPTION OF THE DRAWINGS

FIGS. 1A-B show a model of ordered VlhA switching of MG. (1A) The current working model of ordered VlhA switching of MG in response to the changing respiratory epithelial phenotype during infection. Loose attachment to ciliary cells with VlhA proteins transitions to tight attachment on the cell membrane with both VlhA proteins and the cytadhesins GapA and CrmA, utilizing gliding motility mechanisms. As epithelial phenotypes change, so do their surface molecules, and vlhA gene expression in the bacterium changes to allow for continued adhesion to host cells. (1B) In the presence of a vaccine that contains all of the VlhA antigens that are expressed early in MG infection, phase variation is not allowed to progress (as would happen after vaccination with a bacterin vaccine that only expressed the first VlhA protein when the vaccine is produced). Even the rare bacterial cell that manages to evade antibodies and attach to host cells will be targeted when VlhA switching occurs, as antibodies are present against these subsequent VlhA proteins as well. The addition of GapA and CrmA antigens further helps to ensure that attachment of MG bacteria to host respiratory epithelium is minimized.

FIG. 2 shows the vlhA expression profile of MG extracted directly from tracheas of experimentally infected birds over the course of the 7-day infection as determined through RNA sequencing. Each data point represents an average RPKM value from the results determined for five animals (with the exception of the broth sample). Error bars show standard errors of the means (SEM). Key statistically significant changes between two time points are indicated by paired upper- and lowercase letters for the following genes: vlhA 3.03 (A/a and A′/a′) and vlhA 4.07.1/4.08 (B/b).

FIG. 3A-C show subunit vaccine reduces pathology and bacterial loads after MG challenge. Vaccination/Challenge, HB=Hayflick's Broth, FM1=unadjuvanted subunit vaccine, FM2=adjuvanted vaccine, asterisks (*) indicate statistical significance between indicated groups. (3A) Tracheal thickness. (3B) M. gallisepticum endpoint viable recovery in CCU (Color Changing Unit, a liquid-phase, limiting dilution assessment of bacterial concentration). (3C) M. gallisepticum daily dynamic viable recovery measures in Mean CCU.

FIG. 4A-D show subunit vaccine induces antibody against subunits and to MG. Pooled sera are from negative control (Saline), FM1 (unadjuvanted subunit vaccine), FM2 (adjuvanted vaccine) groups. Asterisks (*) indicate statistical significance between indicated groups. (4A) gapA/crmA coated (IgY 1:1000). (4B) gapA/crmA/vlhA Coated (IgY 1:1000). (4C) VLHA coated (IgY 1:1000). (4D) M. gallisepticum lysate coated (IgY 1:1000).

FIG. 5A-D show subunit vaccine induces antibody against individual phase variable proteins (VlhA). Pooled sera are from negative control (Saline), FM1 (unadjuvanted subunit vaccine), FM2 (adjuvanted vaccine) groups. Asterisks (*) indicate statistical significance between groups. (5A) VLHA 3.03 coated (IgY 1:1000). (5B) VLHA 3.06 coated (IgY 1:1000). (5C) VLHA 4.07 coated (IgY 1:1000). (5D) VLHA 5.05 coated (IgY 1:1000).

The above-described and other features will be appreciated and understood by those skilled in the art from the following detailed description, drawings, and appended claims.

### DETAILED DESCRIPTION

Described herein is a subunit MG vaccine based on the recent assessment of global transcriptomic vlhA gene expression directly from MG populations present on the tracheal mucosa during a 7-day experimental infection in chickens which showed that at given time points, specific vlhA genes were dominant, suggesting a nonstochastic and temporal progression of dominant vlhA gene expression in the colonizing bacterial population. Early-phase VlhA proteins were selected and expressed in E. coli, purified and used in a standard vaccination/challenge experiment in chickens. The vaccine comprises a combination of antigens selected from individual and combinatorial attachment proteins GapA and CrmA, and any one or more early-phase VlhA antigens, such as for example, VlhA 3.03, 3.06, 4.07, 5.05. Additional VlhA antigens, such as 4.08 and tandem repeat variants 4.07.1 and 4.07.6 can optionally be included. The vaccine induced antibody responses against all major components of the subunit vaccine, showing that the vaccine induces immunity, and conferred measures of protective efficacy against virulent MG challenge, e.g., reductions in the tracheal thickness characteristic of MG pathology, and reduction of isolated bacterial loads from the trachea.

Without being bound to a theory, it is believed that immunodominant proteins encoded by members of the variable lipoprotein and hemagglutinin (vlhA) gene family are believed to be important for MG-host interaction, pathogenesis, and immune evasion. As infection progresses, host cells experience denuding of cilia, squamous cell metaplasia, and eventual destruction of the host cell membranes, which triggers a change in response to these alterations and the pathogen expressing a specific set of vlhA genes to best persist in the current environment. We show that chickens vaccinated with the vaccine described herein comprising the selected early phase VlhA antigens, as well as GapA and CrmA generate anti-attachment and anti-VlhA neutralizing antibodies to each of the specific VlhAs dominantly expressed during the initial phase of infection. In the face of such a repertoire of preformed anti-GApA, anti-CrmA and anti-VlhA antibodies resulting from vaccination, MG attempting to infect the host will be assaulted by specific antibodies that will minimize their ability to attach and those that do manage to attach will be met by anti-VlhA antibodies each time it attempts to switch to a successive VlhA. Any residual MG that may possibly remain will be eliminated by the host immune system.

Advantageously, the vaccine maintains a high safety profile, without the possibility of reversion to virulence that can occur with live attenuated vaccines. Further, the vaccine is amenable to rapid modification via the incorporation of additional MG virulence determinants if needed, or protective antigens from other significant avian pathogens such as Mycoplasma synoviae (MS), Mycoplasma meleagridis (MM), and Mycoplasma iowae. The vaccine can be modified very rapidly with relevant gene sequence information from outbreaks or field strains of Mycoplasma gallisepticum.

In an aspect, a subunit Mycoplasma gallisepticum (MG) vaccine comprises one or more recombinant early-phase VlhA antigens 1.01, 1.02, 1.03, 1.04, 1.05, 1.06, 1.07, 2.01, 2.02, 3.02, 3.03, 3.04, 3.05, 3.06, 3.07, 3.08, 3.09, 4.01, 4.02, 4.03, 4.04, 4.05, 4.06, 4.07. 4.07.1. 4.07.3, 4.07.4, 4.07.5, 4.07.6, 4.08, 4.09, 4.10, 4.11, 4.12, 5.02, 5.03, 5.04, 5.05, 5.06, 5.07, 5.08, 5.09, 5.11, 5.12, 5.13, in combination with one or more of GapA, CrmA, or another individual or combinatorial attachment protein. In one aspect, the antigens are from virulent MG strain R_{low} (Genomic sequence GenBank accession no. AE015450), virulent field strain, VA1994, attenuated strain F, or other virulent field strains, or a combination thereof. In another aspect the vaccine is a squalene-based oil-in-water adjuvanted mixture. In one aspect, the adjuvant is a Montanide ISA adjuvant, for example Montanide™ ISA 71R. In another aspect, the vaccine comprises an antigen from another avian pathogen such as Mycoplasma synoviae (MS), Mycoplasma meleagridis (MM), and Mycoplasma iowae.

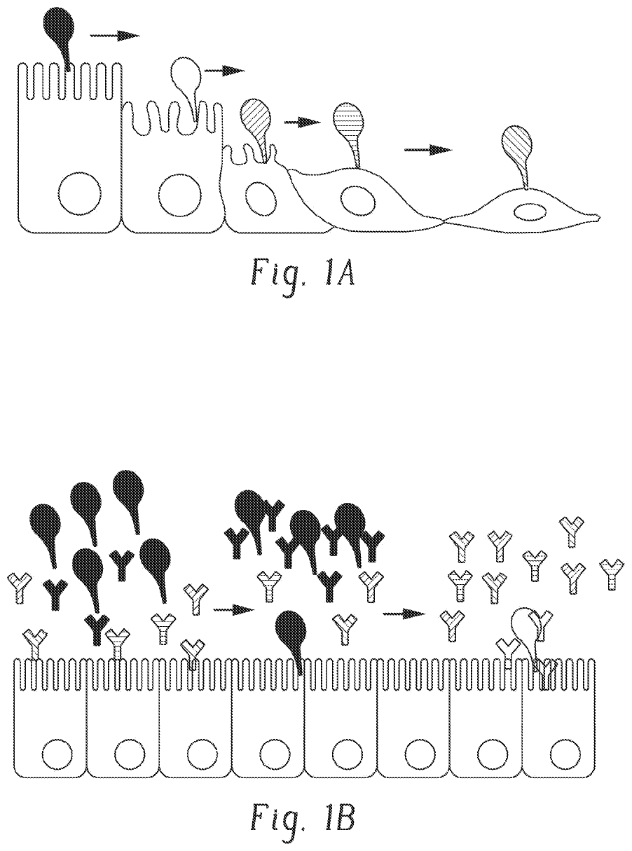

vlhA (variable lipoprotein and hemagglutinin) is a large multigene family that encodes the hemagglutinins/adhesins in MG. The VlhAs expressed from this gene family have been reported to contribute to the pathogen binding to epithelial surfaces. They are phase-variably expressed, antigenically heterogeneous, and are the immunodominant antigens in MG-infected birds. Detailed analysis of the expression of the vlhAs in vivo shows that vlhA phase variation is dynamic throughout the earliest stages of infection and that the pattern of dominant vlhA expression is nonrandom and regulated by previously unrecognized mechanism(s). The current working model of ordered VlhA switching of MG in response to the changing respiratory epithelial phenotype during infection is shown in FIG. 1A, B. Loose attachment to ciliary cells with VlhA proteins transitions to tight attachment on the cell membrane with both VlhA proteins and the cytoadhesins GapA and CrmA, utilizing gliding motility mechanisms. As epithelial phenotypes change, so do their surface molecules, and vlhA gene expression in the bacterium changes to allow for continued adhesion to host cells.

Nucleotide sequences encoding VlhA proteins and amino acid of the native M. gallisepticum VlhA proteins are known and are encompassed in this invention. Presented in Table 1 are the native nucleotide sequences encoding M. gallisepticum VlhA antigens GapA, CrmA, VlhA 3.03, 3.06, 4.07, 4.08, and 5.05, amino acid sequences corresponding to M. gallisepticum VlhA antigens GapA, CrmA, VlhA 3.03, 3.06, 4.07, 4.08, and 5.05, and the E. coli optimized sequences of these antigens used to prepare recombinant M. gallisepticum VlhA antigens for use in a M. gallisepticum vaccine. M. gallisepticum vaccine compositions comprising any of the M. gallisepticum VlhA antigens, or any combination of M. gallisepticum VlhA antigens, are provided herein and can be prepared using the methods provided herein.

In an aspect, and to potentially improve protein expression, proteins can be modified to lack transmembrane domains and express as soluble ‘extracellular’ proteins (EC) in the cell, for example, extracellular-domain GapA and CrmA can be expressed (GapA-EC and CrmA-EC, respectively) in E. coli. For each protein, this entails removal of sequences encoding predicted amino-terminal and carboxyl-terminal intracellular and transmembrane domains, codon optimization for expression in E. coli, and addition of sequences encoding a 10× Histidine tag (His-Tag, SEQ ID NO:50) to the carboxyl terminus.

TABLE 1
SEQUENCES OF M. GALLISEPTICUM ANTIGENS

| | GM native | GM native | DNA sequence | Amino acid seq |
|---|---|---|---|---|
| | nucleotide | amino acid | of recombinant | of recombinant |
| Antigen | sequence | sequence | protein | protein |
| VlhA 3.03 | SEQ ID NO: 1 | SEQ ID NO: 2 | SEQ ID NO: 3 | SEQ ID NO: 4 |
| VlhA 3.06 | SEQ ID NO: 5 | SEQ ID NO: 6 | SEQ ID NO: 7 | SEQ ID NO: 8 |
| VlhA 4.07 | SEQ ID NO: 9 | SEQ ID NO: 10 | SEQ ID NO: 11 | SEQ ID NO: 12 |
| VlhA 4.08 | SEQ ID NO: 13 | SEQ ID NO: 14 | SEQ ID NO: 15 | SEQ ID NO: 16 |
| VlhA 5.05 | SEQ ID NO: 17 | SEQ ID NO: 18 | SEQ ID NO: 19 | SEQ ID NO: 20 |
| CrmA | SEQ ID NO: 21 | SEQ ID NO: 22 | SEQ ID NO: 23 | SEQ ID NO: 24 |
| GapA | SEQ ID NO: 25 | SEQ ID NO: 26 | SEQ ID NO: 27 | SEQ ID NO: 28 |
| CrmA-EC | SEQ ID NO: 21 | SEQ ID NO: 22 | SEQ ID NO: 38 | SEQ ID NO: 39 |
| GapA-EC | SEQ ID NO: 25 | SEQ ID NO: 26 | SEQ ID NO: 40 | SEQ ID NO: 41 |
| VlhA 4.07.1 | SEQ ID NO: 42 | SEQ ID NO: 43 | SEQ ID NO: 44 | SEQ ID NO: 45 |
| VlhA 4.07.6 | SEQ ID NO: 46 | SEQ ID NO: 47 | SEQ ID NO: 48 | SEQ ID NO: 49 |

In one aspect, a recombinant VlhA protein or antigen or a CrmA or a GapA protein or antigen is provided by cloning the nucleic acid sequence, or a variant thereof with 90% or 95% sequence identity, encoding the desired antigen in a vector for expression in a host cell. Examples of expression plasmids with M. gallisepticum antigens are presented in Table 1.1. When the host cell is E. coli, an E. coli modified nucleic acid optimized for expression in E. coli can be used. In another aspect, as long as the antigenic determinant formed by folding of a peptide encoded by said nucleic acid sequence is maintained, a fusion of two or more VlhA proteins and/or a CrmA or a GapA antigens is provided.

TABLE 1.1
Sequence of vectors with M. gallisepticum antigens

| | Cloning vector with | | |
|---|---|---|---|
| Antigen | insert sequence | Inserted gene name and length | Cloning vector |
| VlhA 3.03 | SEQ ID NO: 30 | vlhA_3.03_SJG (1988 bp) | pET-28b(+) |
| VlhA 3.06 | SEQ ID NO: 31 | vlhA_3.06_SJG (2111 bp) | pET-28b(+) |
| VlhA 4.07 | SEQ ID NO: 32 | vlhA_4.07_SJG (2048 bp) | pET-28b(+) |
| VlhA 4.08 | SEQ ID NO: 33 | vlhA_4.08_SJG (2111 bp) | pET-28b(+) |
| VlhA 5.05 | SEQ ID NO: 34 | vlhA_5.05_SJG (1979 bp) | pET-28b(+) |
| CrmA | SEQ ID NO: 35 | crmA_SIG (3221 bp) | pET-28b(+) |
| GapA | SEQ ID NO: 36 | gapA_SJG (3401 bp) | pET-28b(+) |

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques.

Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “% identity”, as used throughout the specification, may for example be calculated as follows. The query sequence is aligned to the target sequence using the CLUSTAL W algorithm (Thompson et al, Nucleic Acids Research, 22:4673-4680 (1994)). A comparison is made over the window corresponding to one of the aligned sequences, for example the shortest. The window may in some instances be defined by the target sequence. In other instances, the window may be defined by the query sequence. The amino acid residues at each position are compared, and the percentage of positions in the query sequence that have identical correspondences in the target sequence is reported as % identity.

The term “vector” as used herein, can refer to a vehicle for carrying or transferring a nucleic acid. Non-limiting examples of vectors include plasmids and viruses (for example, AAV viruses). Vectors for expressing a VlhA antigen, a CrmA or a GapA antigens or expression vectors, are known in the art. The vectors can be used in an E. coli gene expression system for producing the recombinant antigens or proteins. Different gene expression vectors and compatible cell expression systems, for example for expression in SF9 insect cells, can be used and are known in the art. The vector comprises one or more nucleotide sequence encoding the recombinant protein(s) operably linked to an expression control element, such as a promoter, an operator, an enhancer, or other transcriptional or translational control element known to one of skill in the art to influence the expression of the RNA and/or protein products encoded by the polynucleotide within cells. Example promoters include phage T7, SP6 promoter, lactose operon-promoter, tac promoter, SV40 late promoter, SV40 early phase promoter, CMV IE promoter, among others.

In one aspect, the antigen's native signal sequence or leader sequence is removed and exchanged for a signal sequence that allows secretion of the recombinant protein from the host cell. Signal sequences are N-terminal extensions of newly synthesized secretory and membrane proteins. They are usually 16 to 30 amino acid residues in length and comprised of a hydrophilic, usually positively charged N-terminal region, a central hydrophobic domain and a C-terminal region with the cleavage site for signal peptidase. The mycoplasma lipoprotein signal sequence may be removed and replaced with the E. coli lipoprotein signal sequence to ensure soluble and robust expression in E. coli. Other host cells, such as for example Lactococcus lactis, Streptomyces lividans, or Corynebacterium glutamicum, and Bacillus subtilis can be used to produce the recombinant proteins, and secretion signals known in the art for use in a specific expression host cell can be used.

Optionally, a peptide sequence comprises a sequence not present in the antigen as found in nature, such as a tag for purification or a detectable label. The tag may comprise a His tag (e.g., 6 his-tag, SEQ ID NO:29 or a 10 his-tag, SEQ ID NO:50), a maltose binding protein (MBP), a small ubiquitin-like modifier (SUMO), a Glutathione S-transferase (GST), a Streptococcal G protein (SGp), or combinations and/or portions thereof. In some aspects, a histidine tag is added to the N-terminus of the encoded protein with enables easy purification of the expressed protein by affinity purification. The histidine tag can be modified to include a Tobacco Etch Virus (TEV) protease site such to enable removal of the fused histidine tag after protein purification. Other methods for affinity purification and labeling of the expressed protein known to skilled artisans can be used.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)). Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The subunit vaccine can additionally comprise other antigens, such as a gallisepticum adhesion protein A (GapA) and a cytoadhesin-related molecule A (CrmA) as discussed above, or a VhlA antigen from any other selected phases of infection, for example as second early-phase, and any other newly identified M. gallisepticum adhesion, adhesion-related, or surface protein found to interact with host substrates.

In one aspect, a method of making a Mycoplasma gallisepticum (MG) subunit vaccine composition, comprising determining an early phase variable lipoprotein hemagglutinin (VhlA) antigen expression profile from MG infected birds, selecting a first early-phase variable VhlA antigen with a maximum expression at a first time post-infection, selecting a second early-phase VhlA antigen with a maximum expression at a second time post-infection, recombinantly expressing the first and second early-phase VhlA antigens, recombinantly expressing a primary MG adhesion protein A (GapA) antigen, recombinantly expressing a cytoadhesin-related protein MG molecule A (CrmA) protein antigen, and combining the recombinantly expressed first and second early-phase VhlA antigens, the GapA antigen, the CrmA antigen, and an immune-enhancing amount of an adjuvant to provide the vaccine.

In another aspect, a subunit vaccine composition comprises a combination of recombinant early phase VlhA proteins, one or more GapA protein, and/or one or more CrmA protein from one or more mycoplasma strains, from one or more of the four species of mycoplasma infectious in birds, for example, MG, Mycoplasma synoviae (MS), Mycoplasma meleagridis (MM), and Mycoplasma iowae for protection against infection from one or more field strains of mycoplasma. Therefore, a vaccine composition is provided for protection against infection with any of MG, Mycoplasma synoviae (MS), Mycoplasma meleagridis (MM), and Mycoplasma iowae, or a combination thereof.

In yet another aspect, a method for inducing an immune response in birds against infection with a mycoplasma, such as MG, MS, MM, and/or Mycoplasma iowae, or a method of immunizing an avian against infection with MG, MS, MM, and/or Mycoplasma iowae, comprising administering to the bird a vaccine composition described herein comprising antigens selected from one or more of the infectious mycoplasma in an amount sufficient or effective to induce a protective immune response against all of the one or more mycoplasma represented in the vaccine.

In the presence of a vaccine composition containing one or more of the VlhA antigens that are expressed early during mycoplasma infection, phase variation is not allowed to progress (as would happen after vaccination with a bacterin vaccine that only expressed the first VlhA protein when the vaccine is produced). Even the rare mycoplasma bacterial cell that manages to evade antibodies and attach to host cells will be targeted when VlhA switching occurs, as antibodies are present against these subsequent VlhA proteins as well. The addition of one or more of a GapA and/or one or more of a CrmA antigen further helps to ensure that attachment of mycoplasma bacteria to host respiratory epithelium is minimized.

Therefore, in another aspect, a method for protecting birds from the natural progression of mycoplasma infection, e.g. infection by any of MG, MS, MM, and/or Mycoplasma iowae, comprising altering the natural expression pattern of the mycoplasma early phase vlhA genes during early infection stages by administering to the bird a vaccine composition comprising one or more recombinant VlhA antigens, selected based on expression during a phase of infection, e.g. early first or second phase; and an immune-effective amount of an adjuvant, wherein an immune response against the VlhA antigens is mounted in the bird, wherein the expression pattern of the mycoplasma vlhA genes is altered and the progression of mycoplasma disease is halted. In one aspect, the VlhA antigens are early phase antigen selected from VlhA 3.03, VlhA 3.06, VlhA 4.07, VlhA 5.05. Additional VlhA antigens, such as 4.08 and tandem repeat variants 4.07.1 and 4.07.6 can optionally be included.

According to the present invention, a “dose” of a vaccine composition, is a quantity of vaccine composition that is administered at a particular point in time. A “dose” may also be a quantity of vaccine composition that is gradually administered to a bird. According to the present invention, a dose is typically within the range of about 0.25 mL to about 2.0 mL. A dose of subunit vaccine composition that can be administered to a bird in the context of the present invention can be a volume of vaccine composition of about, e.g., 0.25 mL, 0.30 mL, 0.35 mL, 0.40 mL, 0.45 mL, 0.50 mL, 0.55 mL, 0.60 mL, 0.65 mL, 0.70 mL, 0.75 mL, 0.80 mL, 0.85 mL, 0.90 mL, 0.95 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, etc.

In certain embodiments of the present invention, two or more doses of the vaccine composition are administered to a bird at different time points. For example, the present invention includes vaccination methods in which a first dose of vaccine composition is administered to the bird at a first time point, and then a second dose of vaccine composition is administered to the bird at a second time point (e.g., booster vaccination). The second time point may be between 1 and 90 days after the first time point. For example, in methods that involve multiple administrations of the vaccine composition to the animal, the second time point may be 5, 6, 7, 8, 9, 10, 11, 12, 13, 14, 15, 16, 17, 18, 19, 20, 21, 22, 23, 24, 25, 26, 27, 28 or more days after the first time point.

According to the present invention, an amount to induce a protective immune response or “immunologically-effective amount” of the subunit vaccine composition which will induce complete or partial immunity in a treated bird against subsequent challenge with a virulent strain of mycobacteria. Complete or partial immunity can be assessed by observing, either qualitatively or quantitatively, the clinical symptoms of avian mycoplasma infection in a vaccinated bird, such as the tracheal thickness characteristic of MG pathology, and measure of bacterial loads from the trachea as compared to an uninfected bird. Where the clinical symptoms of mycoplasma infection in a vaccinated bird after challenge are reduced, lessened or eliminated as compared to the symptoms observed in an unvaccinated bird after a similar or identical challenge, such as significant reductions in the tracheal thickness characteristic of MG pathology, and also in reduction of isolated bacterial loads from the trachea the amount of vaccine that was administered to the vaccinated, is regarded as an “immunologically-effective amount.”

Exemplary amounts of subunit vaccine that may be regarded as “immunologically-effective amounts”, depending on the size of the bird to which the vaccine is administered, an “immunologically-effective amount” of vaccine may be, e.g., 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 ug/subunit per dose.

In addition to an immunogenically-effective amount of the vaccine compositions of the present invention, the composition may also comprise a pharmacologically acceptable carrier. Exemplary pharmacologically acceptable carriers include water, saline, or phosphate or other suitable buffers. The vaccine compositions may also comprise an oil component. For example, when an oil component is included, the vaccine composition can be formulated as a water-in-oil or oil-in-water emulsion. Also contemplated are double emulsions, often characterized as water-in-oil-in-water emulsions. The oil may help to stabilize the formulation and further function as an adjuvant or enhancer. Suitable oils include, without limitation, white oil, Drakeoil, squalane or squalene, as well as other animal, vegetable or mineral oils, whether naturally-derived or synthetic in origin.

Other exemplary adjuvants include, but are not limited to, aluminum hydroxide or Alum, aluminum phosphate or other metal salts, MPLA, Montanide™ ISA 71R, Montanide™ ISA 71R an oil-based water-in-oil emulsion or oil-in-water emulsion, composed of mineral oil, non-mineral oil, or a composition of both as well as surfactants, cationic liposomes with Toll-like receptor (TLR) 1, 2, 6, 7, and 9/21 ligands, microparticles plus TLR ligands, squalene-in-water emulsion plus TLR ligands, dimethyl dioctadecyl ammonium bromide (DDA), or DDA/trehalose 6,6,9-dibehenate (TDB) liposomes, cationic liposomes with TLR 1, 2, 6, 7, and 9/21 ligands, microparticles plus TLR ligands, squalene-in-water emulsion plus TLR ligands, or Carbopol.

The vaccine compositions of the present invention may, in certain embodiments, contain a lipopolysaccharide, e.g., a bacterial lipopolysaccharide. Bacterial lipopolysaccharide-derived adjuvants may be purified and processed from bacterial sources, or alternatively they may be synthetic, such as 3-O-Deacylated monophosphoryl or diphosphoryl lipid A derived from Salmonella sp., or 3-O-Deacylated monophosphoryl lipid A (3D-MPL). An exemplary form of 3D-MPL is in the form of an emulsion having a small particle size less than 0.2 μm in diameter.

The vaccine compositions of the present invention may, in certain embodiments, also contain saponins. Saponins are steroid or triterpene glycosides widely distributed in the plant and marine animal kingdoms. Saponins are noted for forming colloidal solutions in water which foam on shaking, and for precipitating cholesterol. When saponins are near cell membranes, they create pore-like structures in the membrane which cause the membrane to burst. Haemolysis of erythrocytes is an example of this phenomenon, which is a property of certain, but not all, saponins. Saponins are known as adjuvants in vaccines for systemic administration. The adjuvant and haemolytic activity of individual saponins has been studied in the art. For example, Quil A (derived from the bark of the South American tree Quillaja saponaria Molina), and fractions thereof, particulate structures, termed Immune Stimulating Complexes (ISCOMS), comprising fractions of Quil A are haemolytic and have been used in the manufacture of vaccines. The haemolytic saponins QS21 and QS17 (HPLC purified fractions of Quil A) have been described as potent systemic adjuvants. Other saponins which have been used in systemic vaccination studies include those derived from other plant species such as gypsophila and saponaria.

Additional excipients may also be included in the vaccine compositions of the present invention, including, e.g., surfactants or other wetting agents or formulation aids. Surfactants can include the sorbitan mono-oleate esters (TWEEN® series), as well as the ethylene oxide/propylene oxide block copolymers (PLURONIC® series), as well as others available in the art. Additional non-ionic surfactants include Triton X-45, t-octylphenoxy polyethoxyethanol (Triton™ X-100), Triton™ X-102, Triton™ X-114, Triton™ X-165, Triton™ X-205, Triton™ X-305, Triton™ N-57, Triton™ N-101, Triton™ N-128, Brej 35, polyoxyethylene-9-lauryl ether (laureth 9) and polyoxyethylene-9-stearyl ether (steareth 9).

Other compounds recognized as stabilizers or preservatives may also be included in the vaccine compositions of the present invention. These compounds include, without limitation, carbohydrates such as sorbitol, mannitol, starch, sucrose, dextrin or glucose and the like, as well the preservative formalin, for example.

The vaccine compositions of the present invention may also be formulated as a dry powder, substantially free of exogenous water, which may then be reconstituted by an end user prior to administration. The formulation of the vaccine composition is made to suit the mode of administration.

According to the methods of the invention, any method of administration can be used to administer the vaccine composition to the bird. An exemplary method of administration is subcutaneous delivery. Any device may be used for delivery in the context of the present invention. Intradermal vaccines may also be administered by devices which limit the effective penetration length of a needle into the skin or using a jet injection device which delivers liquid vaccines to the dermis via a liquid jet injector or via a needle which pierces the stratum corneum and produces a jet which reaches the dermis. Also exemplary are ballistic powder/particle delivery devices which use compressed gas to accelerate vaccine in powder form through the outer layers of the skin to the dermis. Additionally, conventional syringes may be used in the classical mantoux method of intradermal administration.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the subunit vaccine compositions to reach their targets.

Suitable birds include, but are not limited to, birds, turkey, chicken, e.g., a 10-week-old chicken, a commercial egg layer or a broiler, pheasant, chukar partridge, peafowl, guinea fowl, pigeon, quail, duck, goose, and psittacine bird. The methods can include in ovo delivery of the composition to an embryo or chick in need thereof.

The invention is further illustrated by the following non-limiting examples.

### EXAMPLES

### Methods

Animals. Four-week-old female specific-pathogen-free White Leghorn chickens (SPAFAS, North Franklin, CT, USA) were received and divided randomly into groups, placed in HEPA-filtered isolators, and allowed to acclimate for 1 week. Non-medicated feed and water were provided ad libitum throughout the experiment. All animal studies were performed in accordance with approved University of Connecticut (UConn) IACUC protocol number A13-001.

Chicken infection. Stocks of M. gallisepticum strain R_{low} (passage 17) were grown overnight at 37° C. in complete Hayflick's medium until mid log phase was reached as indicated by a color shift from red to orange. Bacterial concentrations were determined by the optical density at 620 nm (OD620), and 10-fold serial dilutions were conducted to confirm viable color-changing-unit titers. Bacteria were pelleted by centrifugation at 10,000×g for 10 min and resuspended in Hayflick's complete medium. Chickens were challenged intratracheally as previously described with 1×10⁸ CFU/200 ul.

RNA extraction. Five infected chickens were humanely sacrificed each day for a total of 7 days. After sacrifice, tracheas were excised and total RNA was extracted from each individual trachea by washing the lumen with 1 ml TRIzol reagent (Invitrogen™ Carlsbad, CA, USA). Total RNA was then purified using a Zymo Direct-zol RNA Miniprep kit (Zymo Research Corporation, Irvine, CA, USA), and standard PCR was conducted to ensure that the RNA preparations were free of any DNA. RNA was quality checked using an Agilent 2100 Bioanalyzer (Agilent Technologies, Santa Clara, CA, USA), and high-quality samples with RNA integrity numbers (RIN) of >8 were utilized to construct cDNA libraries. To enrich for bacterial RNA, total RNAs were subjected to a poly(A) depletion step to remove the eukaryotic mRNA using a NEBNext® poly(A) mRNA magnetic isolation module (New England BioLabs®, Ipswich, MA, USA). Briefly, 5 μg of total RNA combined with an equal volume of bead binding buffer was bound to poly(T) oligonucleotide-attached magnetic beads at 65° C. for 5 min. The remaining supernatant was collected, cleaned, and concentrated using a Zymo Clean & Concentrator-25 Kit™ (Zymo Research Corporation®) and eluted in 25 ul RNase-free water. Both prokaryotic rRNA and eukaryotic rRNA were removed from 2.5 μg of poly(A)-depleted RNA using a RiboZero Magnetic Gold Kit™ (Epidemiology) (Illumina™ Inc., San Diego, CA, USA) following the manufacturer's instructions. Each rRNA-depleted RNA sample obtained after cleaning and concentrating with the Zymo Clean&Concentrator-25 Kit™ (ZymoResearch Corporation®) was eluted in 25 ul of RNase-free water, and the samples were used to create a cDNA library.

Illumina sequencing. cDNA libraries were created using an Illumina TruSeq™ stranded mRNA library preparation kit (Illumina Inc.™, San Diego, CA, USA) according to the manufacturer's instructions starting at “Make RFP” (step 14; p. 56) of the Illumina TruSeq™ RNA Sample Preparationv2 (HT) protocol. Briefly, 10 to 400 ng of purified mRNA was fragmented and used to synthesize first-strand cDNA using reverse transcriptase and random hexamer primers. Second-strand cDNA synthesis was performed using dUTP, DNA polymerase, and RNase. The products were amplified by PCR and purified after end repair and adaptor ligations. The cDNA libraries were assessed for quantity using a Qubit 2.0 fluorometer (Invitrogen™) and for correct fragment size (approximately 260 bp) using an Agilent TapeStation 2200® instrument (Agilent Technologies). Libraries were then normalized to 2 nM, pooled, denatured, and sequenced on a NextSeq500™ Sequencing platform (Illumina™ Inc.) using a 75-bp paired-end approach.

RNA-seq analysis. Fastq data were assembled and mapped, and differential gene expression levels were assessed using Rockhopper, an RNA sequencing (RNA-seq) analysis program with algorithms specifically designed for bacterial gene structures and transcriptomes (http://cs.wellesley.edu/˜btjaden/Rockhopper/). Bowtie2 parameters allowing 0 mismatches were used to map sequence reads to the Rlow genomic template. The data were normalized by the standard method of determining the ratio of reads per kilobase per million (RPKM) mapped, allowing for comparisons both within and between samples. The fold change data were determined from the log 2 transformation of RPKM data between two samples. The differential levels of gene expression were determined by pairwise comparisons between the normalized values of expression of a given vlhA gene from two different days. The program-generated P value was used to determine significance of the differential levels of gene expression by calculating q values based on the Benjamini-Hochberg correction with a false-discovery rate of <1%. Differences in expression values were considered significant when the q value was <0.02.

Expression of M. pneumoniae and M. gallisepticum Proteins in E. coli BL21 (DE3) Cells Using Pet-28b(+) Plasmids

Preparation of Calcium Chloride Competent Cells: E. coli BL21 (DE3) cells were streaked on antibiotic free agar plate and grown overnight at 37° C. The next evening, a single colony was selected and used to inoculate 5 mL of antibiotic free liquid broth (ex. LB or SOC) and grown overnight at 37° C. with shaking at 250 rpm on a 14 mL culture tube. The next morning, 2 mL of overnight culture was transferred to 200 mL pre-warmed (37° C.), antibiotic-free liquid broth (ex. LB or SOC) in a sterile 1 L conical flask covered loosely with aluminum foil. The culture was allowed to grow at 37° C., 250 rpm until OD600 is between 0.4 and 0.6 for LB and between 0.8-1 for SOC. (The bacteria are still in their log phase at these OD readings.) Cells were transferred to 4 50-mL centrifuge tubes and placed on ice for 30 minutes to chill. Bacterial cells were centrifuged at 4° C. for 10 minutes at 3000×g (rcf) to pellet them. Supernatant was poured off/aspirated, and all 4 pellets were resuspended in 30 mL of ice cold 0.1 M CaCl₂) and place on ice for 30 minutes. Cells were pelleted by centrifugation for 10 minutes at 4° C. at 3000×g (rcf). Supernatant was removed and the pellet was resuspended in 3 mL of ice cold 0.1M CaCl₂) containing 15% glycerol. Tubes were kept on ice.

Heat Shock Transformation: To a 50 μL aliquot, 1-5 uL (or 10 pg-100 ng) of plasmid was added. Cells were incubated on ice for 30 minutes. Bacteria was heat shocked by placing tubes in the thermocycler block at 45° C. for 45 seconds and then tubes were placed on ice for 2 minutes. Cells were transferred to 950 uL pre-warmed antibiotic-free SOC broth in 14 mL culture tube and incubated at 37° C. with shaking at 200 rpm for 1 hour for outgrowth, plated at 1:10 and 1:100 dilutions (100 μL and 10 uL) on pre-warmed agar plates containing appropriate antibiotic at the appropriate working concentration (ex. 100 μg/mL ampicillin, 60 μg/mL kanamycin) and incubated at 37° C. overnight (12-16 hrs).

Transformation Efficiency was calculated by using the following formula:

$$TE = ( \# of transformants ) / ( amount of DNA ( ug ) spread on agar plate )$$

$$TE = ( \# of colonies on plate ) / ( ng of DNA {plated}^{*} ) \times ( 1000 ng ) / 1 ug = CFU / ug of DNA$$

the measurement “ng of DNA plated” refers to amount of DNA plated onto each agar plate, calculated using the following equation:

$$ng of DNA plated = ( volume of plasmid used in uL \times the concentration of DNA in ng / ul ) \times ( ( volume plated ) / ( total reaction volumn ) )$$

Protein expression was assayed by SDS-PAGE gel fractionation. For quick and dirty SDS-PAGE sample prep: 1 mL uninduced and 1 mL induced culture of the same clone was pelleted, resuspend in 100 μL of 2×Laemlii Buffer with BME and boiled for 10 minutes at 99° C. Samples were loaded onto gel and lysates compared for protein expression

### Sample Large Scale Induction Protocol—Using LB Medium

A positive clone was selected and used it to inoculate a starter culture of 10 mL LB containing appropriate antibiotic which was allowed to grow overnight at 37° C. with shaking at 250 rpm. The next morning, 5 mL of starter culture was transferred into pre-warmed 100 mL of LB containing appropriate antibiotic to expand the culture by growth at 37° C. with shaking at 250 rpm until OD 600 is roughly 0.55-0.6. Once the culture has reached appropriate OD600, the bacteria was pelleted by centrifugation at 3500×g (rcf) for 20 minutes and the supernatant was discarded.

Room Temperature (20° C.) induction: Pellet was resuspended in 100 mL room temperature, antibiotic-free expression medium. IPTG was added to a final concentration of 0.1 to 1.0 mM and incubated overnight (12-18 hours) at 20° C. with shaking at 250 rpm.

Cold Induction: Pellet was resuspended in 100 mL room temperature, antibiotic-free expression medium. IPTG was added to a final concentration of 0.1 to 1.0 mM and induced for 24-48 hours at 16° C. with shaking at 250 rpm.

Alternatively, the induction culture was grown in the presence of 3% ethanol until desired OD 600 value. Resuspend pellets in induction/expression medium containing 1%, 2% or 3% ethanol) were induce for 48-72 hours at 16° C.

37° C. Induction: Pellet was resuspended in 100 mL room temperature, antibiotic-free expression medium. IPTG was added to a final concentration of 0.1 to 1.0 mM and induced for 3-4 hours at 37° C. with shaking at 250 rpm. After induction, cells were collected by centrifuging at 10000×g (rcf) for 15 minutes at 4° C.

### Protein Extraction—Using BPER Reagent for a 50 mL Pelleted Induced Culture

Cells were resuspended in 5 mL B-PER reagent and mixed well to homogenize the solution. 100 uL PMSF protease inhibitor and protease inhibitor cocktail was added, along with 100 uL lysozyme. The solution was mixed well and incubated at RT for 15 minutes with gentle agitation (rocking or shaking). The cells were lysed by running the solution through a 24 G needle 20 times. DNA was sheared to make solution less viscous by sonicating 3× for 30 second increments making sure to keep solution cool in the process. The solution was centrifuged at 16000×g (rcf) for 30 minutes to pellet insoluble proteins and inclusion bodies. The supernatant containing clarified lysate with soluble proteins was collected.

Purifying insoluble inclusion bodies: For inclusion body purification, 5 mL of B-PER reagent was added to the pellet (insoluble fraction generated in step above), resuspended and then lysozyme (100 ul of 10 mg/ml stock) was added to the suspension and incubated for 15 minutes at RT. 15 mL of 1:10 diluted B-PER reagent was added to the suspension and inclusion bodies were collected by centrifugation at 160000×g for 45 minutes. The pellet was resuspended in 20 mL of 1:10 diluted B-PER reagent and sonicated for 30 seconds and centrifuged as above to collect inclusion bodies. This step was repeated two more times. After discarding the supernatant from the wash steps above, the purified inclusion bodies were dissolved in denaturing agents (ie. 1 M-8 M urea in PBS pH 7.4) and the proteins further purified on Ni-NTA column.

### HisPur Ni-NTA Purification of His-Tagged Recombinant Proteins (3 mL Columns)

Material Preparation: Depending on the specific protein, buffers may require optimization. The table below provides buffers with different imidazole concentrations. The total volume can be adjusted depending on the resin volume used. For most proteins, the following buffer concentrations can be used.

Equilibration Buffer: 10 mM imidazole in PBS; Wash Buffer: 25 mM imidazole in PBS; Elution Buffer: 250 mM imidazole in B-PER.

Regeneration Buffer: MES Buffer: 20 mM 2-(N-morpholine)-ethanosulfonic acid, 0.1 M NaCl; pH 5.0; or MOPS Buffer: 20 mM 2-(N-morpholine)-propanosulfonic acid, 0.1 M NaCL; pH 5.0.

TABLE 2

| Imidazole Final | 10X PBS | 2M imidazole | Water |
|---|---|---|---|
| Concentration | (mL) | (uL) | (mL) |
| 10 | 1 | 50 | 8.95 |
| 25 | 1 | 125 | 8.875 |
| 40 | 1 | 200 | 8.8 |
| 60 | 1 | 300 | 8.7 |
| 75 | 1 | 375 | 8.625 |
| 150 | 1 | 750 | 8.25 |
| 200 | 1 | 1000 | 8 |
| 250 | 1 | 1250 | 7.75 |
| 500 | 1 | 2500 | 6.5 |

Purifications are performed at RT or in a cold room at 4° C. Sample was prepared by mixing protein extract with Equilibration buffer at a 1:1 ratio. The bottom tab was removed from the HisPur Ni-NTA column by gently twisting and the column placed into a 50 mL centrifuge tube and centrifuged at 700×g (rcf) for 2 minutes to remove storage buffer. The column was equilibrated with two resin-bed volumes of Equilibration buffer (6 mL). The prepared protein extract was added to the column (6 mL at a time) and allowed to enter the resin bed. The sample was incubated with gentle rocking/shaking for 30 minutes, after which, 6 more mL of sample was added, and incubation steps were repeated. At the end of the last incubation step, the column was centrifuged at 700×g for 2 minutes and flow-through collected for further analysis and purification. The resin was washed with two resin bed volumes of Wash buffer. The column was then placed into a clean 50 mL tube and two resin bed volumes of Elution buffer were added and allowed to gravity flow until the column stopped dripping, making sure to collect the eluate and collected fractions were analyzed via SDS-PAGE. Imidazole was removed for downstream applications using gel filtration desalting columns (e.g., Thermo Scientific Zeba™ Spin Desalting Columns).

Mycoplasma sequences were codon-optimized for expression in E. coli. Certain codons were further modified to remove the premature UGA stop codons that would otherwise encode tryptophan. UGA is a stop codon in other bacteria but encodes for tryptophan in mycoplasmas. Cloning restriction sites within the gene sequences were removed. The mycoplasma lipoprotein signal sequence of the VlhAs was removed and replaced with the E. coli Lpp signal sequence to ensure soluble and robust expression in E. coli. A TEV protease cleavage site was added to each protein just prior to its C-terminus hexa-histidine tag.

Mycoplasma gallisepticum Subunit Vaccine Preparation

Expression of Recombinant Proteins in E. coli: Recombinant M. gallisepticum gapA, crmA and vlhA proteins were generated in E. coli BL21 (DE3) following their transformation with the pET28b(+) plasmid containing the appropriate constructs (Table 1). The M. gallisepticum strain R (low) complete genome is available at GenBank AE015450.2 For plasmid generation, the DNA sequences for M. gallisepticum strain R (low) proteins gapA (MGA_0934, SEQ ID NO:25), crmA (MGA_0939, SEQ ID NO:21), vlhA 3.03 (MGA_0380, SEQ ID NO:1), vlhA 3.06 (MGA_0388, SEQ ID NO:5), vlhA 4.07 (MGA_0977, SEQ ID NO: 9), vlhA 4.08 (MGA_0979, SEQ ID NO:13), and vlhA 5.05 (MGA_1245, SEQ ID NO: 17) were codon optimized for E. coli expression. Additionally, DNA sequences encoding the M. gallisepticum lipoprotein signal sequence were replaced with the lipoprotein signal sequence of the E. coli Lpp lipoprotein in order to enhance expression and improve solubility in E. coli BL21 (DE3). The codon optimized DNA sequences were also screened for recognition sequences of XhoI and NcoI restriction enzymes, and adjustments were made to the sequence to remove these sites while retaining the appropriate amino acid codon. The sequences were then submitted to Biomatik-Gene and Peptide Synthesis (Wilmington, Delaware, USA) for synthesis and subcloning into the pET-28b(+) plasmid using NcoI as the 5′ restriction site, and XhoI as the 3′ restriction site to avoid the addition of a N-terminus His-Tag while retaining a TEV protease cleavable C-terminus His-Tag. Chemically competent E. coli BL21 (DE3) cells were transformed with the constructs using a standard heat-shock transformation protocol and were plated on LB-agar plates containing 60 μg/mL Kanamycin to select for successfully transformed bacteria. The plates were grown overnight at 37° C., and 5 clonal colonies per construct were selected, proliferated and stockpiled for further analysis. Individual clones were streaked on LB plates containing 60 μg/mL kanamycin and grown overnight at 37° C. Clonal colonies were selected, inoculated onto LB Broth with 60 μg/mL Kanamycin, and grown until OD 600=0.8. At this point, the bacteria were pelleted by centrifugation at 5000×g for 10 minutes then resuspended in fresh antibiotic free medium and incubated for another hour at 37° C. with shaking at 250 rpm on an orbital shaker/incubator. After the one-hour incubation, recombinant protein expression was induced according to the conditions outlined in the table below:

TABLE 3
RECOMBINANT PROTEIN EXPRESSION

| | | | Induction | |
|---|---|---|---|---|
| | Recombinant | IPTG | Temperature | |
| Construct | Protein | Concentration | (° C.) | Additives |
| pEgapA | r-gapA | 0.5 mM | 24 | 3% EtOH |
| pEcrmA | r-crmA | 0.5 mM | 18 | 3% EtOH |
| pEvlhA303 | r-vlhA 3.03 | 0.5 mM | 16 | N/A |
| pEvlhA306 | r-vlhA 3.06 | 0.5 mM | 16 | N/A |
| pEvlhA407 | r-vlhA 4.07 | 0.5 mM | 16 | N/A |
| pEvlhA505 | r-vlhA 5.05 | 0.5 mM | 16 | N/A |

Following confirmation of expression via SDS-PAGE analysis, large scale cultures were induced overnight to maximize protein production. After overnight induction of protein expression, the cultures were pelleted via centrifugation at 12,000×g (rcf) for 15 minutes and frozen at −80° C. for at least 24 hours. The frozen pellets were then resuspended and lysed using B-PER Bacterial Protein Extraction Reagent (Thermo Fisher Scientific™, Waltham, MA, USA), containing lysozyme and Halt Protease Inhibitor Cocktail (Thermo Fisher Scientific™, Waltham, MA, USA), vigorously vortexed, sonicated on ice for four 30 second intervals to shear the DNA, then centrifuged at 15,000×g (rcf) to pellet insoluble proteins. Recombinant proteins were purified from the clarified lysates (supernatants) which were run through pre-equilibrated HisPur Ni-NTA spin columns (Thermo Fisher Scientific™ Waltham, MA, USA) to bind 6×His-tagged recombinant proteins, washed 3× and eluted with 250 mM imidazole. The proteins were then precipitated via Ammonium Sulfate precipitation and desalted using the Zeba Spin Desalting columns (Thermo Fisher Scientific™, Waltham, MA, USA). Proteins were quantified using the protein assay kit on a Qubit 2.0 Fluorometer (Thermo Fisher Scientific™, Waltham, MA, USA) according to manufacturer instructions, and frozen back at −20° C. until vaccine formulation.

Vaccine Formulations: Three vaccine formulations (FM0, FM1 and FM2) were prepared. FM0 consisted solely of 200 uL physiological saline per dose that is used as a fluid vehicle for vaccine antigen delivery. FM1 consisted of 10 ug recombinant gapA+10 ug recombinant crmA proteins for a total of 20 ug recombinant M. gallisepticum proteins and 10 μg of each recombinant vlhA (vlhA 3.03, 3.06, 4.07 and 4.08) for a total of 60 μg of recombinant M. gallisepticum proteins in 200 uL physiological saline per vaccine dose. FM2 consisted of FM1 protein components (60 ug) in 100 uL physiological saline and 100 uL Addavax™ adjuvant (Invivogen, San Diego, CA, USA) per vaccine dose.

Animals: Four-week-old, female, specific pathogen free, white leghorn chickens (SPAFAS, North Franklin, CT) were used for this pilot experiment. Birds were divided into groups of 10 birds and housed in separate 120×60 in pens. Birds were acclimated for 1 week prior to experimentation in accordance with the approved Geary IACUC protocol A18-057. Non-medicated feed (Blue Seal, Waltham, MA) and water was provided ad libitium via automatic feeders throughout the experimental protocol.

Vaccination/Challenge experiments: Following the one-week acclimation period, chickens were bled via the wing vein on day-2, then subcutaneously vaccinated with each vaccine formulation on day 0 (prime) and day 21 (boost). Chickens were then bled again on day 40, then intratracheally inoculated with 200 ul of sterile Hayflick's Broth (Negative Control) or 200 μL of Hayflick's Broth containing 1×10⁸ CFU (8.34×107 viable CCU) M. gallisepticum Rlow on day 42. Two weeks after challenge (day 56), the chickens were humanely sacrificed and tracheal segments taken for M. gallisepticum isolation, histological processing, and tracheal washes in order to assess the protective efficacy of the vaccine constructs.

Gross and histopathological examination: Following humane euthanasia, chicken tracheas were excised from the glottis and larynx to the syrinx at the level of bifurcation, then bisected. From the resulting bi-segments, 1 cm thick sections were taken for subsequent mycoplasma culture/isolation, histological processing, and the remnant tracheal segments were washed for immunological examination (FIG. 1A, B). Airsacs and visceral organs were closely examined for gross lesions during the necropsies. For histopathology, three 1 cm thick annular segments from the cranial, middle and caudal thirds of the trachea were sampled and fixed in 10% neutral buffered formalin, giving three representative segments that were used for histopathological assessment. These tissues were fixed for 48 hours, trimmed, embedded in paraffin blocks, sectioned at 5 μm then stained with hematoxylin and eosin according to standard histological protocols. The slides were then blinded, tracheal mucosal thickness measured, and further histopathological examinations performed by a Board-Certified Anatomic Pathologist.

Collection of sera and tracheal washes for immunoglobulin detection: After collection of blood, samples were incubated at room temperature for 45 minutes to allow for clotting, then centrifuged at 1700×g (rcf) for 20 minutes in serum separation tubes (BD, Franklin Lakes, NJ). Serum was collected from the clotted blood and gel separator using a P1000 micropipettor fitted with filter tips. The serum was heat inactivated by incubation at 56° C. for 30 minutes, then frozen at −20° C. until further analysis.

Collection of samples for mycoplasma isolation: One-centimeter-thick rings from the cranial, medial and caudal regions of the trachea were pooled into 3 mL of Hayflicks's broth, vortexed vigorously, and incubated for 3 hours at 37° C. Following incubation, the cultures were resuspended by gentle pipetting, and filtered into 3 mLs of fresh Hayflick's broth through 0.45 μm filters to remove contaminating eubacteria. The filtrates were then used to perform serial dilutions in 96-well plates and incubated to estimate titers as endpoint color changing units.

Enzyme-linked Immunosorbent Assays (ELISAs) for Immunoglobulin Characterization: 96-well Nunc Polysorp (Penfield, NY, USA), were coated with 0.5 ug of antigen per well overnight at 4° C. Following coating incubation, ELISA plates were blocked with 0.2 ug Bovine Serum Albumin in 0.2% PBS-Tween-20 (PBST) for 1 hour at room temperature. Following blocking, plates were washed 3 times with 0.2% PBST, and probed with serum from study animals at a 1:1000 dilution for 1 hour at room temperature. Following incubation with serum, the plates were washed 3 times with 0.2% PBST, and probed with (1:30,000 diluted) Horseradish Peroxidase Conjugated Rabbit Anti-Chicken IgY (whole molecule) (Millipore Sigma, St. Louis, MO, USA) for 1 hour at room temperature. Plates were washed 3 times with 0.2% PBST, then developed with 1-Step Ultra TMB-ELISA Substrate Solution (Thermo Fisher Scientific™, Waltham, MA, USA) for 11 minutes, then the reaction stopped by the addition of TMB Stop Solution (Thermo Fisher Scientific™, Waltham, MA, USA). Plates were then read at OD 450 nm using a Cytation™ 5 Plate Reader (Biotek™, Winooski, VT, USA).

Dot-Blot Immunoassay: Dot-blots were performed to assess the reactivity of sera from vaccinated animals with native M. gallisepticum proteins. Briefly, 0.12 ug in 20 μL of Tris-NaCl buffer of M. gallisepticum protein derived from whole cell lysate were spotted onto 0.45 μm Amersham Protran Nitrocellulose membranes which were then allowed to dry at room temperature for 30 minutes. After drying, the membranes were blocked with 0.2 ug Bovine Serum Albumin in 0.2% PBS-Tween-20 (PBST) for 1 hour at room temperature with gentle shaking at 50 rpm on an orbital shaker. Following blocking, the membrane was washed 3× for 5 minutes with PBST, then probed with a 1:3000 dilution of pre-immunization or post-immunization study animal serum (animals from individual groups were pooled) for 1 hour at room temperature with gentle shaking. Following incubation with animal sera, the membranes were washed 3× for 5 minutes with PBST, then probed with (1:30,000 diluted) Horseradish Peroxidase Conjugated Rabbit Anti-Chicken IgY (whole molecule) (Millipore Sigma™, St. Louis, MO, USA) for 1 hour at room temperature with gentle shaking. The membranes were then washed 3× for 5 minutes with PBST, then developed with the Chromogenic Pierce DAB substrate Kit (Thermo Fisher Scientific™, Waltham, MA, USA) that forms an insoluble brown-colored precipitate when reacting with HRP conjugated antibodies. Membranes were allowed to develop for 10 minutes, the reaction stopped, and membranes photographed.

### Example 1: vlhA Gene Family Expression During Infection in Chickens

Our previous work demonstrated that both of the primary cytadhesin molecules, gallisepticum adhesion protein A (GapA) and the cytadhesin-related molecule A (CrmA) are required for effective MG cytadherence. Importantly, antibodies to GapA can reduce cytadherence to host cells in vitro by approximately 80%. Our previous data has shown that GapA and CrmA are consistently expressed at high levels, both in vitro and in chickens in vivo (data not shown). Both GapA and CrmA are conserved across all of the poultry strains sequenced to date, making the inclusion of both of these proteins in a subunit vaccine essential.

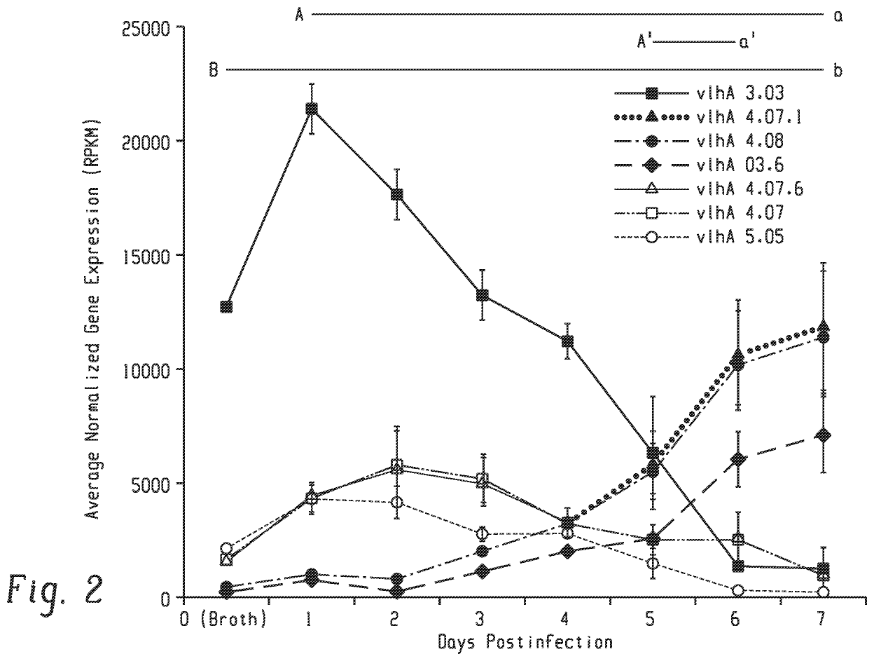

Immunodominant proteins encoded by members of the variable lipoprotein and hemagglutinin (vlhA) gene family are believed to be important for MG-host interaction, pathogenesis, and immune evasion. We assessed global transcriptomic vlhA gene expression directly from MG populations present on the tracheal mucosa during a 7-day experimental infection in chickens and noted that, at given time points, specific vlhA genes were dominant, suggesting a nonstochastic and temporal progression of dominant vlhA gene expression in the colonizing bacterial population (FIG. 2, and data not shown).

Data shows that early phase variation is driven, in part, by the alterations in the cellular architecture of the trachea during infection. As the infection progresses, host cells experience denuding of cilia, squamous cell metaplasia, and eventual destruction of the host cell membranes. It is possible that MG vlhA expression is changing in response to these alterations and the pathogen is expressing a specific set of vlhA genes to best persist in the current environment. Over the course of infection, there was a 34-fold total decrease in vlhA 3.03 expression, with the largest change being a 4.5-fold decrease between days 5 and 6 post-infection (FIG. 2). While expression of vlhA 3.03 decreased early in infection, expression of vlhA 4.07 and its tandem repeat variant, vlhA 4.07.6, began to increase as early as 1 day post-infection. These expression levels peaked at 2 days post-infection (3.7-fold higher than input culture), before decreasing over the remainder of the 7-day infection. Similarly, as vlhA 4.07 and 4.07.6 decreased in expression, vlhA 4.08 and its tandem repeat, vlhA 4.07.1, showed increased expression until peaking at 7 days post-infection with 31-fold-higher expression than the levels of input cultures. Finally, there was an initial increase in vlhA 5.05 expression on 1 day post-infection, followed by decreasing expression through the time course. The vlhA expression profile observed here at day 7 post-infection was consistent with that of a previous experiment (data not shown). Collectively, these changes in vlhA gene expression were not random and appeared to be coordinated, as similar vlhA profiles were seen in independently infected birds at each time point postinfection.

The consistent pattern of vlhA expression provides the basis for inclusion of specific VlhAs in a subunit vaccine and may be the reason that current MG bacterin vaccines are poorly protective (given their lack of vlhA switching). Not to be bound by a theory, it is believed that the vaccinated chickens will generate anti-VlhA neutralizing antibodies to each of the specific VlhAs dominantly expressed during the initial phase of infection. In the face of such a repertoire of preformed anti-VlhA antibodies, MG will be assaulted by specific antibodies each time it attempts to switch to a successive VlhA. With a combination of these and the anti-GapA and anti-CrmA antibodies, any residual MG will be eliminated by the host innate immune system.

Importantly, the phase variable and immunodominant VlhA proteins of MG too have been shown to be protective antigens. Prior to the discovery of the vlhAs in MG, Saito et al. demonstrated that chicken anti-29 kDa peptide serum inhibited the growth of MG in vitro and partial protection to chickens from challenge with virulent MG. It was later determined that this peptide is a portion of a VlhA, demonstrating the role for VlhAs as protective antigens.

### Example 2: Vaccination/Challenge in Chickens

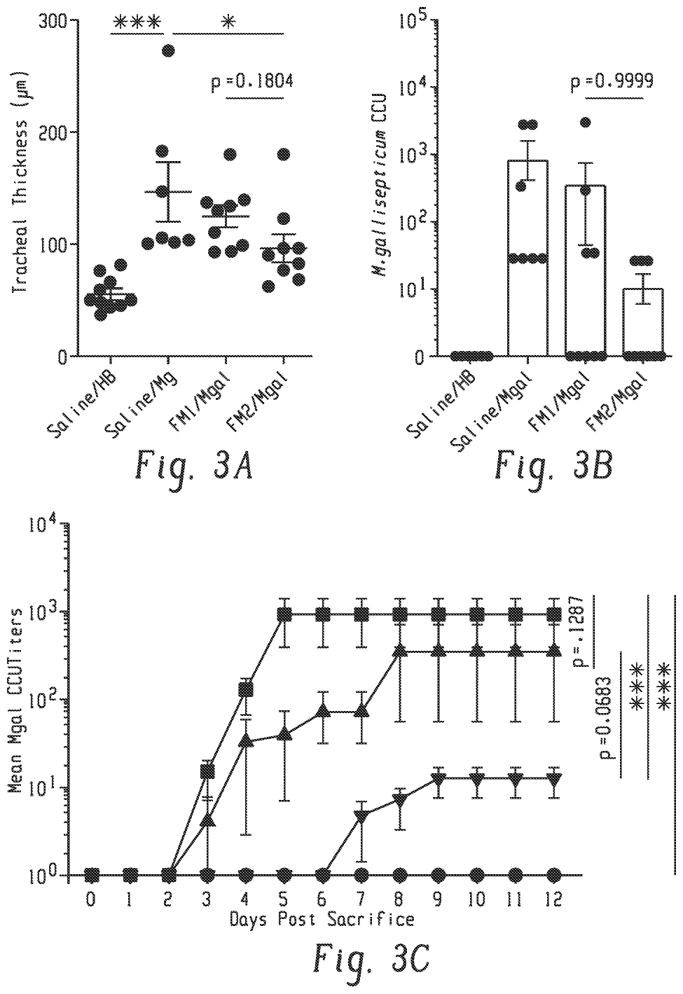

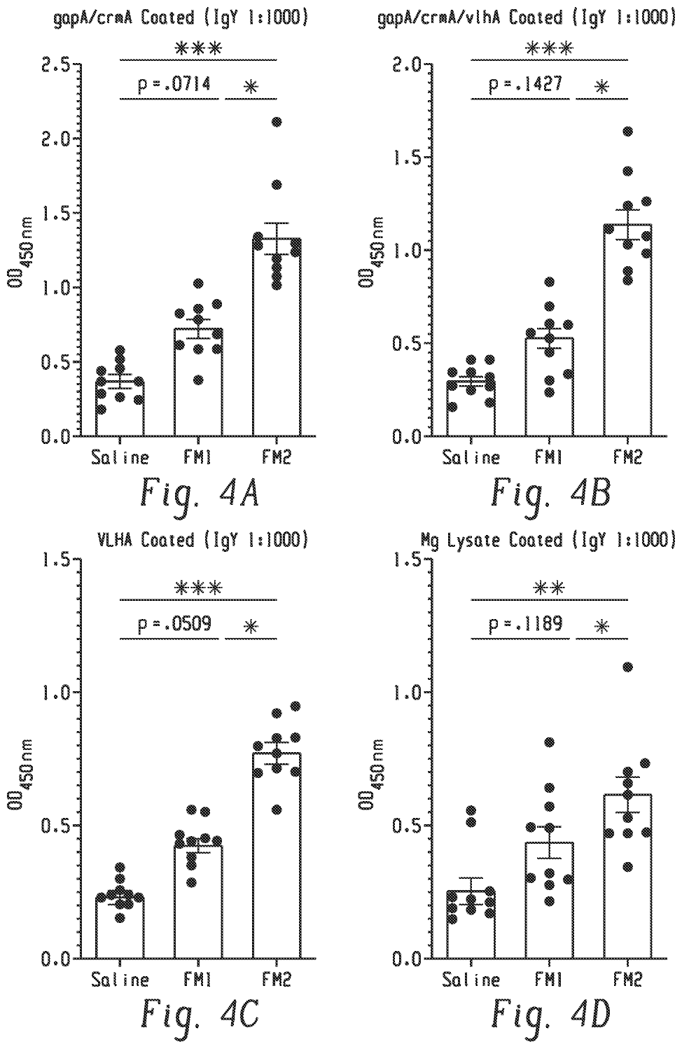

Key attachment proteins (GapA and CrmA) and early-phase VlhA proteins were expressed in E. coli, purified, and used in a standard vaccination/challenge experiment in chickens. Subunit mixtures (10 ug/subunit=60 ug total) were injected SQ either as pure protein or in conjunction with commercial adjuvant (Addavax™). Overall, subunit (FM1) and adjuvanted subunit (FM2) formulations demonstrated protective effects relative to non-vaccinated (‘Saline”) controls. FIG. 3A indicated that subunit vaccines approached or reached significant reductions in the tracheal thickness characteristic of MG pathology, and also in reduction of isolated bacterial loads from the trachea, taken at either endpoint (FIG. 3B) or dynamic timepoints (FIG. 3C, titer indicating growth in vitro in days post-isolation). In addition, our vaccine induced antibody responses against all major components of the subunit vaccine and notably against whole-MG lysates (FIG. 4A-D).

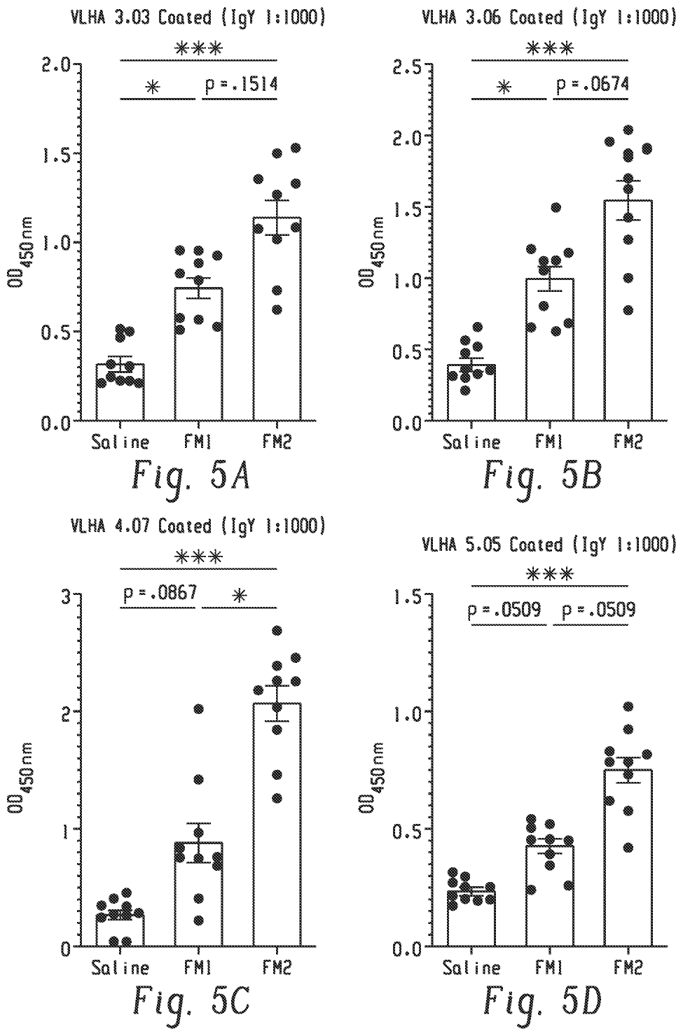

Antibody responses were detected against each of the specific VlhaA protein subunits used in the formulation (FIG. 5A-D). Together, the antibody, pathological, and microbiological data indicate that formulations of an attachment protein/VlhA subunit vaccine induces immunity and confers measures of protective efficacy against virulent MG challenge.

### Example 3: Vaccine Optimization

Assays for optimizing the vaccine, such as dose adjustments, adjuvants, route of administration, schedule of administration, or varying combinations of antigens are in progress.

A subunit MG vaccine based on recent knowledge of essential virulence determinants and the vlhA gene family expression profile is provided. The vaccine has superior efficacy and design flexibility not available with the commercially available bacterin (MG-Bac®) and maintains a high safety profile and without the possibility of reversion to virulence that can occur with live attenuated vaccines. The vaccine is amenable to rapid modification via the incorporation of additional MG virulence determinants if needed, or protective antigens from other significant avian pathogens.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A Mycoplasma gallisepticum (MG) subunit vaccine comprising recombinant gallisepticum adhesion protein A (GapA) antigen, recombinant cytoadhesin-related molecule A (CrmA) protein antigen, one or more recombinantly produced early phase variable lipoprotein hemagglutinin (VhlA) antigen; and an immune enhancing amount of an adjuvant.

2. The vaccine of claim 1, wherein the one or more recombinantly produced VhlA antigen is VlhA 3.03, VlhA 3.06, VlhA 4.07, or VlhA 5.05.

3. The vaccine of claim 1, comprising all of the recombinantly produced VhlA antigens, VlhA 3.03, VlhA 3.06, VlhA 4.07, and VlhA 5.05.

4. The vaccine of claim 1, further comprising one or more of VlhA 4.08, VlhA 4.07.1 and VlhA 4.07.6 antigens.

5. The vaccine of claim 1, wherein one or more of the recombinant gallisepticum GapA, CrmA, or VlhA antigen comprises a peptide sequence not present in the antigen as found in nature, such as a deletion, a tag for purification or a detectable label.

6. The vaccine of claim 5, wherein the tag comprises a His tag, a maltose binding protein (MBP), a small ubiquitin-like modifier (SUMO), a Glutathione S-transferase (GST), a Streptococcal G protein (SGp), or combinations and/or portions thereof.

7. The vaccine of claim 6, wherein the His tag comprises SEQ ID NO 29.

8. The vaccine of claim 1, wherein the adjuvant is a squalene-based oil-in water nano-emulsion.

9. The vaccine of claim 1, wherein the adjuvant is Alum, monophospholipid A, an oil-based water-in-oil emulsion or oil-in-water emulsion, cationic liposomes with Tolllike receptor (TLR) 1, 2, 6, 7, and 9/21 ligands, microparticles plus TLR ligands, squalene-in-water emulsion plus TLR ligands, or dimethyl dioctadecyl ammonium (DDA) bromide, or DDA/trehalose 6,6,9-dibehenate (DDA/TDB) liposomes.

10. The vaccine of claim 2, wherein the GapA antigen comprises a sequence having 90% identity to amino acid SEQ ID NO:28 or comprises a sequence having 90% identity to amino acid SEQ ID NO:41; the CrmA antigen comprises a sequence having 90% identity to amino acid SEQ ID NO:24 or comprises a sequence having 90% identity to amino acid SEQ ID NO: 39; the VlhA 3.03 antigen comprises a sequence having 90% sequence identity to amino acid SEQ ID NO:4; the VlhA 3.06 antigen comprises a sequence having 90% sequence identity to SEQ ID NO:8; and the VhlA 5.05 antigen comprises a sequence having 90% identity to SEQ ID NO:20.

11. The vaccine of claim 4, wherein the VlhA 4.08 antigen comprises a sequence having 90% sequence identity to SEQ ID NO: 16, the VlhA 4.07 antigen comprises a sequence having 90% sequence identity to SEQ ID NO: 12, the VlhA 4.07.1 antigen comprises a sequence having 90% identity to SEQ ID NO:45, and the VlhA 4.07.6 antigen comprises a sequence having 90% identity to SEQ ID NO:49.

12. A method of immunizing an avian against infection with Mycoplasma gallisepticum comprising administering to the avian the vaccine of claim 1, in an amount sufficient to induce a protective immune response.

13. The method of claim 12, wherein the administrating is subcutaneous.

14. The method of claim 13, wherein the subcutaneous administration is at day 0 and repeated on day 28.

15. The method of claim 12, wherein the avian is chicken, turkey, pheasant, chukar partridge, peafowl, guinea fowl, pigeon, quail, duck, goose, or psittacine bird.

16. The method of claim 15, wherein the chicken is a commercial egg layer or a broiler.

17. The method of claim 15, wherein the chicken is 10 weeks old.

18. A method for protecting birds from the natural progression of Mycoplasma gallisepticum (MG) infection, comprising altering the natural expression pattern of the MG early phase vlhA genes during early infection stages by administering to the bird a composition comprising one or more recombinant VlhA antigens selected from VlhA 3.03, VlhA 3.06, VlhA 4.07, and VlhA 5.05; and an immune-effective amount of an adjuvant, wherein an immune response against the VlhA antigens is mounted in the bird, wherein the expression pattern of the MG Vlha genes is altered and the progression of MG disease is halted.

19. The method according to claim 18, wherein the composition further comprises one or more of VlhA 4.08, VlhA 4.07.1 and VlhA 4.07.6 antigens.

20. A method of making a Mycoplasma gallisepticum (MG) subunit vaccine, comprising
determining an early phase variable lipoprotein hemagglutinin (VlhA) antigen expression profile from MG infected birds,
selecting a first early phase variable VlhA antigen with a maximum expression at a first time post-infection,
selecting a second early phase VlhA antigen with a maximum expression at a second time post-infection,
recombinantly expressing the first and second early phase VlhA antigens, recombinantly expressing a primary MG adhesion protein A (GapA) antigen, recombinantly expressing a cytadherence-related protein MG molecule A (CrmA) protein antigen, and
combining the recombinantly expressed first and second early phase VhlA antigens, the GapA antigen, the CrmA antigen, and an immune enhancing amount of an adjuvant to provide the vaccine.


SEQUENCE LISTING (50 sequences)

SEQ ID NO: 1 - DNA, 1938 bp, Mycoplasma gallisepticum
  source 1..1938: mol_type = genomic DNA; note = vlhA 3.03 native DNA sequence
gtgaagagaaaaaacatattaaagtttgttagtttattaggtattggttcgtttgtaatgttagctgcagctagttgtactacaccaacacctaatccaaatcctcctagtggtggtatgaatggtggagatactaatccaggtgatggacaaggtatgatgaatgctgcatctcaagaattagctgctgcaagaatggggttaactactgtatttgattctaaagctaagaatcttggattgtatgttgactacaaaaaaacacaggacactctaacaaaagcatatgatgctgctaagactgttcttgataattcatcatcaacaacacaaaatcttaatgaggctaagactagattagaaactgcaattagaacagctgctactagtaaacaaacctttgatgaacaacatgccgagttagtaaaagtttatgaagaattaaaaacgactttaagtaatgaaactgctactttagctccatatgcagattcgcaatatgctggaattaaaatgcatttaagtgggctatatgatgcaggtaaagccattactactaaaacgttagagccagttgaaggggatccacttacagcagatgttgtaatgatggctaatactaagattgttgaagcaattaaagatgaagtattaaatcctcaaaaagaaaacgcaacaaaactagctgatagttttgttaagcaagtattagtaaaagaaaaaattacaggagttgaagaagctcataataaagcacaacctgctaactacagttttgtaggttacagcgttgatataactggaactgttactggacaaacaagtattcctaactgagattatgctcagagaaccattttcacaaacggtgatgaacctagaagcatttctaatactcctgctgatggtcaaactatggtacaacctttatctaatgtgtcttgaatttatagtttagctggcactggtgctaaatacactttagagtttacttattatggaccatcaacaggttatttatatttcccttataagttagttaatactagtgatcaaatgaaactaggtttagaatataaattaaatgatgcgactgaaccaagcgcgatcacttttggtagtgagcaaacaatgaatggtaaaactccaactgttaatgatattaatgtagctaaagttactttagctaacttaaaatttggttcaaacaaaattgagtttagtgttccagcagaaaaagtaagtccgatgatcggtaatatgtatctttcttctagtcctaataactggaataagatctatgatgatatttttggtaatagtgttacaactgaaaataatagaacaataatttcagttgatgctttaaatggttatagtttagcttcagattgatcaacatatattgctgaatacagtggtgcaggtttaacattaaacgaccaagcaaaacctaatgaaaaatactatttaattgggtatgtgggtggaactggtgctcgtaatgatatgatggttccaaagaacaatgttcaaaaattcccattagcaaacaacacaagtaatagaaactacgtattctatgtaaatgctccaaaagcaggtgattattatattaaaggagtgttcgcttcaggagttcatagtgatcttaaattcagtaccggagatatgtcttctaataatgtaactgtaaaacaattgtttacaggtaatttaactactacattaagaacctttgatacctctgcaacaacagaatctacccgagttacaactgatcctactaataaaaagacgttaactctagtagaaggattaaataagatagtagttagtggaactactgaaaatattggtgctccaaattttggatatttagaatttattcttaatgaaacccaacctgaaactactaatgtttcaaatccatcttag

SEQ ID NO: 2 - protein, 645 aa, Mycoplasma gallisepticum
  source 1..645: note = vlhA 3.03 native amino acid sequence
MKRKNILKFVSLLGIGSFVMLAAASCTTPTPNPNPPSGGMNGGDTNPGDGQGMMNAASQELAAARMGLTTVFDSKAKNLGLYVDYKKTQDTLTKAYDAAKTVLDNSSSTTQNLNEAKTRLETAIRTAATSKQTFDEQHAELVKVYEELKTTLSNETATLAPYADSQYAGIKMHLSGLYDAGKAITTKTLEPVEGDPLTADVVMMANTKIVEAIKDEVLNPQKENATKLADSFVKQVLVKEKITGVEEAHNKAQPANYSFVGYSVDITGTVTGQTSIPNWDYAQRTIFTNGDEPRSISNTPADGQTMVQPLSNVSWIYSLAGTGAKYTLEFTYYGPSTGYLYFPYKLVNTSDQMKLGLEYKLNDATEPSAITFGSEQTMNGKTPTVNDINVAKVTLANLKFGSNKIEFSVPAEKVSPMIGNMYLSSSPNNWNKIYDDIFGNSVTTENNRTIISVDALNGYSLASDWSTYIAEYSGAGLTLNDQAKPNEKYYLIGYVGGTGARNDMMVPKNNVQKFPLANNTSNRNYVFYVNAPKAGDYYIKGVFASGVHSDLKFSTGDMSSNNVTVKQLFTGNLTTTLRTFDTSATTESTRVTTDPTNKKTLTLVEGLNKIVVSGTTENIGAPNFGYLEFILNETQPETTNVSNPS

SEQ ID NO: 3 - DNA, 1974 bp, synthetic construct
  source 1..1974: mol_type = other DNA; note = DNA sequence of recombinant VlhA 3.03
atgaaagcgaccaaactggtgctgggcgcggtgattctgggcagcaccctgctggcgggctgcagcagcaacgatggcattcgcggcagcattcgcaccaccccgaccccgaacccgaacccgccgagcggcggcatgaacggcggcgataccaacccgggcgatggccagggcatgatgaacgcggcgagccaggaactggcggcggcgcgcatgggcctgaccaccgtgtttgatagcaaagcgaaaaacctgggcctgtatgtggattataaaaaaacccaggataccctgaccaaagcgtatgatgcggcgaaaaccgtgctggataacagcagcagcaccacccagaacctgaacgaagcgaaaacccgcctggaaaccgcgattcgcaccgcggcgaccagcaaacagacctttgatgaacagcatgcggaactggtgaaagtgtatgaagaactgaaaaccaccctgagcaacgaaaccgcgaccctggcgccgtatgcggatagccagtatgcgggcattaaaatgcatctgagcggcctgtatgatgcgggcaaagcgattaccaccaaaaccctggaaccggtggaaggcgatccgctgaccgcggatgtggtgatgatggcgaacaccaaaattgtggaagcgattaaagatgaagtgctgaacccgcagaaagaaaacgcgaccaaactggcggatagctttgtgaaacaggtgctggtgaaagaaaaaattaccggcgtggaagaagcgcataacaaagcgcagccggcgaactatagctttgtgggctatagcgtggatattaccggcaccgtgaccggccagaccagcattccgaactgggattatgcgcagcgcaccatttttaccaacggcgatgaaccgcgcagcattagcaacaccccggcggatggccagacgatggtgcagccgctgagcaacgtgagctggatttatagcctggcgggcaccggcgcgaaatataccctggaatttacctattatggcccgagcaccggctatctgtattttccgtataaactggtgaacaccagcgatcagatgaaactgggcctggaatataaactgaacgatgcgaccgaaccgagcgcgattacctttggcagcgaacagaccatgaacggcaaaaccccgaccgtgaacgatattaacgtggcgaaagtgaccctggcgaacctgaaatttggcagcaacaaaattgaatttagcgtgccggcggaaaaagtgagcccgatgattggcaacatgtatctgagcagcagcccgaacaactggaacaaaatttatgatgatatttttggcaacagcgtgaccaccgaaaacaaccgcaccattattagcgtggatgcgctgaacggctatagcctggcgagcgattggagcacctatattgcggaatatagcggcgcgggcctgaccctgaacgatcaggcgaaaccgaacgaaaaatattatctgattggctatgtgggcggcaccggcgcgcgcaacgatatgatggtgccgaaaaacaacgtgcagaaatttccgctggcgaacaacaccagcaaccgcaactatgtgttttatgtgaacgcgccgaaagcgggcgattattatattaaaggcgtgtttgcgagcggcgtgcatagcgatctgaaatttagcaccggcgatatgagcagcaacaacgtgaccgtgaaacagctgtttaccggcaacctgaccaccaccctgcgcacctttgataccagcgcgaccaccgaaagcacccgcgtgaccaccgatccgaccaacaaaaaaaccctgaccctggtggaaggcctgaacaaaattgtggtgagcggcaccaccgaaaacattggcgcgccgaactttggctatctggaatttattctgaacgaaacccagccggaaaccaccaacgtgagcaacccgagcgaaaacctgtattttcagagc

SEQ ID NO: 4 - protein, 658 aa, synthetic construct
  source 1..658: note = AA sequence of recombinant VlhA 3.03 protein
MKATKLVLGAVILGSTLLAGCSSNDGIRGSIRTTPTPNPNPPSGGMNGGDTNPGDGQGMMNAASQELAAARMGLTTVFDSKAKNLGLYVDYKKTQDTLTKAYDAAKTVLDNSSSTTQNLNEAKTRLETAIRTAATSKQTFDEQHAELVKVYEELKTTLSNETATLAPYADSQYAGIKMHLSGLYDAGKAITTKTLEPVEGDPLTADVVMMANTKIVEAIKDEVLNPQKENATKLADSFVKQVLVKEKITGVEEAHNKAQPANYSFVGYSVDITGTVTGQTSIPNWDYAQRTIFTNGDEPRSISNTPADGQTMVQPLSNVSWIYSLAGTGAKYTLEFTYYGPSTGYLYFPYKLVNTSDQMKLGLEYKLNDATEPSAITFGSEQTMNGKTPTVNDINVAKVTLANLKFGSNKIEFSVPAEKVSPMIGNMYLSSSPNNWNKIYDDIFGNSVTTENNRTIISVDALNGYSLASDWSTYIAEYSGAGLTLNDQAKPNEKYYLIGYVGGTGARNDMMVPKNNVQKFPLANNTSNRNYVFYVNAPKAGDYYIKGVFASGVHSDLKFSTGDMSSNNVTVKQLFTGNLTTTLRTFDTSATTESTRVTTDPTNKKTLTLVEGLNKIVVSGTTENIGAPNFGYLEFILNETQPETTNVSNPSENLYFQS

SEQ ID NO: 5 - DNA, 2067 bp, Mycoplasma gallisepticum
  source 1..2067: mol_type = genomic DNA; note = Mycoplasma gallisepticum VlhA 3.06 nucleotide sequence
gtgaaaagaaaaaacattttaaagtttgttagtttattaggtattggttcgtttgtaatgttagcagctgctagctgtactcaagctagcactccaactccaactccaactcctaccccaactcctaccccaacacctaaccctgaaccaaagccaaatcctggtggtggtgacacgaataatcctcctagtggtggtatgaatggcgtagatactaatccaggtggtggacaaggcatgatgaatgctactaaccaggaattagtgaatgctaaaaaagctttaagtgatttaattggtggagaatctaagactgttgaattatatgcagactatgcaaaaattaaagctgacttgacttctgcgtacgcagttgctaaaacgacttcagatagttcaacatcaactttagatcaagttaaaacagcaacatcaactttacaaactgctattaatactgctgctagtgacaaagaaaaatttgatcaacaaaatagccaattattaatggcatacaaagttttaaaagacactcttaataaaaaagaagctatagtaatgtcattaaatcaagaaaaatatagcgcaattttgagtgaaattaatgcagcttcttcaacagctgaagaaatcgttaaacaaacattgaatccagtgaatggtaatcttccagttgtagctacacttaatgctgaaaacaccaaaatcctagaagcaataaaagaagaaaaaattaattcagaaaaaagtaatgctgatctgtttgcaaattatcaattgtataaattggatagaactaaattaatgagtgaaggttctaataatacgaaacaaccaggaaattatagctttgtagcttatgctagtgacatagcaagtccaaactgaaattttgcacaaagaacagtttgaacagcagatagtagaacttgaaccagccctttacctaataatttacaaaactctgctcctttaacagatgtgtcatgaatctacactttaagcgggacaggagctaaatatacactaacatttgattattatggcccacaaactggttatttatactttccttataagttggttaaaactagtgataaagtcgggcttcaatataaattaaatcaagcagatcctgtagcgattcagtttagtgaagcggctgctactgcttcagctccagctgaaacagatggtagacaagaatcagctgaaactacaactgctaatgagaaagttaacccaatgccatcagttaatactattaatgttgctaaagtgactttatctaatttaaagtttggttctaacacaattgaatttagtgttccaatggatcaagacaatatgaataaagttgcgccaatgattggtaatatctatattacttcatctaatgatgaagcaaataaaaagcaaatttacgatagtatttttggaaacacttcatcacaaactgctagccaaacatctgttagtgttgatctattaaaaggatatagtcttacaactagttgaagcacatatattcgtcaatttactggtttaacaggtaatggtgtacaaacctctgaccttgtttatttaattggtttgattgatggtagccagaatcgtacagttgcaagcggtccaatgaatattaaaaatattcctaatatgaatggcgctacaagaacattcacaatatatgcaaatgcaccagtaaacgggaactatcacataagtggtgcatatttacaaggagcgaatcaagcaagaagtctaaaattctcaaccggtacaagtggcagtaataatgaagttacagtcactggtttaaaacaaaaaaattgaaccacattaggtcactttgatactaagatgactagcactactgtttcaggaacaggtacaccaatgaaaagaactctaaccttaaatcaaggtttaaacaagattgttgtatcaggagtaaacaatggcgatacaccatttataggtaatctaacatttactttagaaagcaatcaaacgattagtgaacaagttgagcctagttctgctactgcagaaaagaatatataa

SEQ ID NO: 6 - protein, 688 aa, Mycoplasma gallisepticum
  source 1..688: note = Mycoplasma gallisepticum VlhA 3.06 protein amino acid sequence
MKRKNILKFVSLLGIGSFVMLAAASCTQASTPTPTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNLQNSAPLTDVSWIYTLSGTGAKYTLTFDYYGPQTGYLYFPYKLVKTSDKVGLQYKLNQADPVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLTTSWSTYIRQFTGLTGNGVQTSDLVYLIGLIDGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNI

SEQ ID NO: 7 - DNA, 2103 bp, synthetic construct
  source 1..2103: mol_type = other DNA; note = Recombinant VlhA 3.06 nucleotide sequence
atgaaagcgaccaaactggtgctgggcgcggtgattctgggcagcaccctgctggcgggctgcagcagcaaccatggcattcgcggcagcattcgcacccaggcgagcaccccgaccccgaccccgaccccgaccccgaccccgaccccgaccccgaacccggaaccgaaaccgaacccgggcggcggcgataccaacaacccgccgagcggcggcatgaacggcgtggataccaacccgggcggcggccagggcatgatgaacgcgaccaaccaggaactggtgaacgcgaaaaaagcgctgagcgatctgattggcggcgaaagcaaaaccgtggaactgtatgcggattatgcgaaaattaaagcggatctgaccagcgcgtatgcggtggcgaaaaccaccagcgatagcagcaccagcaccctggatcaggtgaaaaccgcgaccagcaccctgcagaccgcgattaacaccgcggcgagcgataaagaaaaatttgatcagcagaacagccagctgctgatggcgtataaagtgctgaaagataccctgaacaaaaaagaagcgattgtgatgagcctgaaccaggaaaaatatagcgcgattctgagcgaaattaacgcggcgagcagcaccgcggaagaaattgtgaaacagaccctgaacccggtgaacggcaacctgccggtggtggcgaccctgaacgcggaaaacaccaaaattctggaagcgattaaagaagaaaaaattaacagcgaaaaaagcaacgcggatctgtttgcgaactatcagctgtataaactggatcgcaccaaactgatgagcgaaggcagcaacaacaccaaacagccgggcaactatagctttgtggcgtatgcgagcgatattgcgagcccgaactggaactttgcgcagcgcaccgtgtggaccgcggatagccgcacctggaccagcccgctgccgaacaacctgcagaacagcgcgccgctgaccgatgtgagctggatttataccctgagcggcaccggcgcgaaatataccctgacctttgattattatggcccgcagaccggctatctgtattttccgtataaactggtgaaaaccagcgataaagtgggcctgcagtataaactgaaccaggcggatccggtggcgattcagtttagcgaagcggcggcgaccgcgagcgcgccggcggaaaccgatggccgccaggaaagcgcggaaaccaccaccgcgaacgaaaaagtgaacccgatgccgagcgtgaacaccattaacgtggcgaaagtgaccctgagcaacctgaaatttggcagcaacaccattgaatttagcgtgccgatggatcaggataacatgaacaaagtggcgccgatgattggcaacatttatattaccagcagcaacgatgaagcgaacaaaaaacagatttatgatagcatttttggcaacaccagcagccagaccgcgagccagaccagcgtgagcgtggatctgctgaaaggctatagcctgaccaccagctggagcacctatattcgccagtttaccggcctgaccggcaacggcgtgcagaccagcgatctggtgtatctgattggcctgattgatggcagccagaaccgcaccgtggcgagcggcccgatgaacattaaaaacattccgaacatgaacggcgcgacccgcacctttaccatttatgcgaacgcgccggtgaacggcaactatcatattagcggcgcgtatctgcagggcgcgaaccaggcgcgcagcctgaaatttagcaccggcaccagcggcagcaacaacgaagtgaccgtgaccggcctgaaacagaaaaactggaccaccctgggccattttgataccaaaatgaccagcaccaccgtgagcggcaccggcaccccgatgaaacgcaccctgaccctgaaccagggcctgaacaaaattgtggtgagcggcgtgaacaacggcgataccccgtttattggcaacctgacctttaccctggaaagcaaccagaccattagcgaacaggtggaaccgagcagcgcgaccgcggaaaaaaacattgaaaacctgtattttcagagc

SEQ ID NO: 8 - protein, 701 aa, synthetic construct
  source 1..701: note = Amino acid sequence of VlhA 3.06 recombinant protein
MKATKLVLGAVILGSTLLAGCSSNHGIRGSIRTQASTPTPTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNLQNSAPLTDVSWIYTLSGTGAKYTLTFDYYGPQTGYLYFPYKLVKTSDKVGLQYKLNQADPVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLTTSWSTYIRQFTGLTGNGVQTSDLVYLIGLIDGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNIENLYFQS

SEQ ID NO: 9 - DNA, 2004 bp, Mycoplasma gallisepticum
  source 1..2004: mol_type = genomic DNA; note = Mycoplasma gallisepticum vlhA 4.07 native DNA sequence
gtgaagagaaaaaacattttaaagtttgttagtttattaggtattggttcgtttgtgatgttggctgcagctagttgtacttcagcaactacaccaacacctaacccaacaccaaagccagatccaatgccaaatcctggtggtggtatgaatggtggaaatactaatcgagggaatggtggtggtatgatgggggataatcctaatcctgggaacactacaccagaacaacaattagcagctgctagaaaaacgctaacggatctacttggtacagaaaataccaacgttgccttttatgctgattatgccaagattcaaagcactttaaacgctgcttatatgacagctaaaactgcatcagaaaatacaagtgcatctttagaaaatctaagatctgcatcaactacactacaagcagctattgataaagctgctagtgataaacgtgcatttgatagtgctaatcaatctttagtaactgcatataataatttaaaaactacactgcaatttaaaacaactagtttagaagaattgtctgaaaataaatatagtggtattaaaaatcacttaagtaaactgtttgatacaggtagtgcaataacagctaaaacattagatcctactagtggtgaaagacctactttagagaaagtgaacgaagctaacaatagcattaaaatggctattagtcctgaatcgttgaagaaatgaaaagataatgcagacaaatttaatgagtttgagaaaaacccgttgtctaaagagaagttatctacaggaacagattcagctcataatcaagaacaaccagcaaactatagttttgtaggttatagcgttgatgtaactggaactactacagctcctgcttcagaaaaagctcttcttaactgaaactatgctcagagaacgatttttacaagtggtgatcaaccaactaagattgatactcctgcagcttcagacgaagctccagctcaacctttatcaaatgtttcttgaatttatagtttagcaggtaatggagctaaatatacgctagatttcacctactatggaccatcaacaggttatttatatttcccttataagttagttaaaaatagtgatagtgttggtttacaatataagttaaataatagtcctactgaaacagcaatcgcttttggtaatatgcaaaatgccaacggtccaactgcaacagtggatagtattaatatagctaaagttactttatctaatttaaattttggagcaaacaaaattgaattcagtgttccttcagaaaaagtagctccgatgattggtaatatgtatcttacttcatcagctgatgaagctaataaacaaaaaatcgaaaatagtatttttggtaatagtgttacaactgaaaataatagaacaataatttcagttgatgctttaagtggttatagtttagcttctgattgatcaacatttattggaaggtatgcagaccaacagttaaccttagatggaaaaatgatgcgtgatcaaaaatattatctaatcggttatgttggtggtaatactggcgctcgtaatgttactggaataacagaaaaaacaaacgagcaaagatctccagtagttagtactccaaaaaaaagaacttatacattctatgtaaatgctccaaaagcaggtgcttattatattaaaggagtgttcgcttcaaaggttcgtagagatcttaaattcagtaccggagatatgtcttctaataatgtaactatacaacaattgtctacaaataatttaactacattaagaacctttgacacctctgcaacaacaggagctacccgagttacaactgtttctgatagaaagacattaactctagtagagggattaaataagatagtagttagtggagctactgcagataatggtaatgctccaaattttggatatttagaatttattcttaatgaaacccaacctgaaactactaatgtttcaaatccatcttag

SEQ ID NO: 10 - protein, 667 aa, Mycoplasma gallisepticum
  source 1..667: note = mycoplasma gallisepticum VlhA 4.07 native aa sequence
MKRKNILKFVSLLGIGSFVMLAAASCTSATTPTPNPTPKPDPMPNPGGGMNGGNTNRGNGGGMMGDNPNPGNTTPEQQLAAARKTLTDLLGTENTNVAFYADYAKIQSTLNAAYMTAKTASENTSASLENLRSASTTLQAAIDKAASDKRAFDSANQSLVTAYNNLKTTLQFKTTSLEELSENKYSGIKNHLSKLFDTGSAITAKTLDPTSGERPTLEKVNEANNSIKMAISPESLKKWKDNADKFNEFEKNPLSKEKLSTGTDSAHNQEQPANYSFVGYSVDVTGTTTAPASEKALLNWNYAQRTIFTSGDQPTKIDTPAASDEAPAQPLSNVSWIYSLAGNGAKYTLDFTYYGPSTGYLYFPYKLVKNSDSVGLQYKLNNSPTETAIAFGNMQNANGPTATVDSINIAKVTLSNLNFGANKIEFSVPSEKVAPMIGNMYLTSSADEANKQKIENSIFGNSVTTENNRTIISVDALSGYSLASDWSTFIGRYADQQLTLDGKMMRDQKYYLIGYVGGNTGARNVTGITEKTNEQRSPVVSTPKKRTYTFYVNAPKAGAYYIKGVFASKVRRDLKFSTGDMSSNNVTIQQLSTNNLTTLRTFDTSATTGATRVTTVSDRKTLTLVEGLNKIVVSGATADNGNAPNFGYLEFILNETQPETTNVSNPS

SEQ ID NO: 11 - DNA, 2040 bp, synthetic construct
  source 1..2040: mol_type = other DNA; note = Recombinant VlhA 4.07 DNA sequence
atgaaagcgaccaaactggtgctgggcgcggtgattctgggcagcaccctgctggcgggctgcagcagcaatcatggcattcgcggcagcattcgcaccagcgcgaccaccccgaccccgaacccgaccccgaaaccggatccgatgccgaacccgggcggcggcatgaacggcggcaacaccaaccgcggcaacggcggcggcatgatgggcgataacccgaacccgggcaacaccaccccggaacagcagctggcggcggcgcgcaaaaccctgaccgatctgctgggcaccgaaaacaccaacgtggcgttttatgcggattatgcgaaaattcagagcaccctgaacgcggcgtatatgaccgcgaaaaccgcgagcgaaaacaccagcgcgagcctggaaaacctgcgcagcgcgagcaccaccctgcaggcggcgattgataaagcggcgagcgataaacgcgcgtttgatagcgcgaaccagagcctggtgaccgcgtataacaacctgaaaaccaccctgcagtttaaaaccaccagcctggaagaactgagcgaaaacaaatatagcggcattaaaaaccatctgagcaaactgtttgataccggcagcgcgattaccgcgaaaaccctggatccgaccagcggcgaacgcccgaccctggaaaaagtgaacgaagcgaacaacagcattaaaatggcgattagcccggaaagcctgaaaaaatggaaagataacgcggataaatttaacgaatttgaaaaaaacccgctgagcaaagaaaaactgagcaccggcaccgatagcgcgcataaccaggaacagccggcgaactatagctttgtgggctatagcgtggatgtgaccggcaccaccaccgcgccggcgagcgaaaaagcgctgctgaactggaactatgcgcagcgcaccatttttaccagcggcgatcagccgaccaaaattgataccccggcggcgagcgatgaagcgccggcgcagccgctgagcaacgtgagctggatttatagcctggcgggcaacggcgcgaaatataccctggattttacctattatggcccgagcaccggctatctgtattttccgtataaactggtgaaaaacagcgatagcgtgggcctgcagtataaactgaacaacagcccgaccgaaaccgcgattgcgtttggcaacatgcagaacgcgaacggcccgaccgcgaccgtggatagcattaacattgcgaaagtgaccctgagcaacctgaactttggcgcgaacaaaattgaatttagcgtgccgagcgaaaaagtggcgccgatgattggcaacatgtatctgaccagcagcgcggatgaagcgaacaaacagaaaattgaaaacagcatttttggcaacagcgtgaccaccgaaaacaaccgcaccattattagcgtggatgcgctgagcggctatagcctggcgagcgattggagcacctttattggccgctatgcggatcagcagctgaccctggatggcaaaatgatgcgcgatcagaaatattatctgattggctatgtgggcggcaacaccggcgcgcgcaacgtgaccggcattaccgaaaaaaccaacgaacagcgcagcccggtggtgagcaccccgaaaaaacgcacctataccttttatgtgaacgcgccgaaagcgggcgcgtattatattaaaggcgtgtttgcgagcaaagtgcgccgcgatctgaaatttagcaccggcgatatgagcagcaacaacgtgaccattcagcagctgagcaccaacaacctgaccaccctgcgcacctttgataccagcgcgaccaccggcgcgacccgcgtgaccaccgtgagcgatcgcaaaaccctgaccctggtggaaggcctgaacaaaattgtggtgagcggcgcgaccgcggataacggcaacgcgccgaactttggctatctggaatttattctgaacgaaacccagccggaaaccaccaacgtgagcaacccgagcgaaaacctgtattttcagagc

SEQ ID NO: 12 - protein, 680 aa, synthetic construct
  source 1..680: note = Recombinant VlhA 4.07 protein aa sequence
MKATKLVLGAVILGSTLLAGCSSNHGIRGSIRTSATTPTPNPTPKPDPMPNPGGGMNGGNTNRGNGGGMMGDNPNPGNTTPEQQLAAARKTLTDLLGTENTNVAFYADYAKIQSTLNAAYMTAKTASENTSASLENLRSASTTLQAAIDKAASDKRAFDSANQSLVTAYNNLKTTLQFKTTSLEELSENKYSGIKNHLSKLFDTGSAITAKTLDPTSGERPTLEKVNEANNSIKMAISPESLKKWKDNADKFNEFEKNPLSKEKLSTGTDSAHNQEQPANYSFVGYSVDVTGTTTAPASEKALLNWNYAQRTIFTSGDQPTKIDTPAASDEAPAQPLSNVSWIYSLAGNGAKYTLDFTYYGPSTGYLYFPYKLVKNSDSVGLQYKLNNSPTETAIAFGNMQNANGPTATVDSINIAKVTLSNLNFGANKIEFSVPSEKVAPMIGNMYLTSSADEANKQKIENSIFGNSVTTENNRTIISVDALSGYSLASDWSTFIGRYADQQLTLDGKMMRDQKYYLIGYVGGNTGARNVTGITEKTNEQRSPVVSTPKKRTYTFYVNAPKAGAYYIKGVFASKVRRDLKFSTGDMSSNNVTIQQLSTNNLTTLRTFDTSATTGATRVTTVSDRKTLTLVEGLNKIVVSGATADNGNAPNFGYLEFILNETQPETTNVSNPSENLYFQS

SEQ ID NO: 13 - DNA, 2067 bp, Mycoplasma gallisepticum
  source 1..2067: mol_type = genomic DNA; note = VlhA 4.08 native DNA sequence
gtgaaaagaaaaaacattttaaagtttgttagtttattaggtattggttcgtttgtaatgttagcagctgctagctgtactcaagctagcactccaactccaactccaactccaactccaactccaaccccaacacctaaccctgaaccaaagccaaatcctggtggtggtgacacgaataatcctcctagtggtggtatgaatggcgtagatactaatccaggtggtggacaaggcatgatgaatgctactaaccaggaattagtgaatgctaaaaaagctttaagtgatttaattggtggagaatctaagactgttgaattatatgcagactatgcaaaaattaaagctgacttgacttctgcgtacgcagttgctaaaacgacttcagatagttcaacatcaactttagatcaagttaaaacagcaacatcaacgttacaaactgctattaatactgctgctagtgacaaagaaaaatttgatcaacaaaatagccaattattaatggcatacaaagttttaaaagacactcttaataaaaaagaagctatagtaatgtcattaaatcaagaaaaatatagcgcaattttgagtgaaattaatgcagcttcttcaacagctgaagaaatcgttaaacaaacattgaatccagtgaatggtaatcttccagttgtagctacacttaatgctgaaaacaccaaaatcctagaagcaataaaagaagaaaaaattaattcagaaaaaagtaatgctgatctgtttgcaaattatcaattgtataaattggatagaactaaattaatgagtgaaggttctaataatacgaaacaaccaggaaattatagctttgtagcttatgctagtgacatagcaagtccaaactgaaattttgcacaaagaacagtttgaacagcagatagtagaacttgaaccagccctttacctaataattcacaaaactctgctcctttaacagatgtgtcatgaatctacactttaagcgggacaggagctaaatatacactaacatttgattattatggcccacaaactggttatttatactttccttataagttggctaaaaccagtgataaagtcgggcttcaatataaattaaatcaagcagattctgtagcgattcagtttagtgaagcggctgctactgcttcagctccagctgaaacagatggtagacaagaatcagctgaaactacaactgctaatgagaaagttaacccaatgccatcagttaatactattaatgttgctaaagtgactttatctaatttaaagtttggttctaacacaattgaatttagtgttccaatggatcaagacaatatgaataaagttgcgccaatgattggtaatatctatattacttcatctaatgatgaagcaaataaaaagcaaatttacgatagtatttttggaaacacttcatcacaaactgctagccaaacatctgttagtgttgatctattaaaaggatatagtcttgcaactagttgaagcacatatattcgtcaatttactggtttaacaggtaatggtgtacaaacctctgaccttgtttatttaattggtttgattggtggtagccagaatcgtacagttgcaagcggtccaatgaatattaaaaatattcctaatatgaatggcgctacaagaacattcacaatatatgcaaatgcaccagtaaacgggaactatcacataagtggtgcatatttacaaggagcgaatcaagcaagaagtctaaaattctcaaccggtacaagtggcagtaataatgaagttacagtcactggtttaaaacaaaaaaattgaaccacattaggtcactttgatactaagatgactagcactactgtttcaggaacaggtacaccaatgaaaagaactctaaccttaaatcagggtttaaacaagattgttgtatcaggagtaaacaatggcgatacaccatttataggtaatctaacatttactttagaaagcaatcaaacgattagtgaacaagttgagcctagttctgctactgcagaaaagaatatataa

SEQ ID NO: 14 - protein, 688 aa, Mycoplasma gallisepticum
  source 1..688: note = VlhA 4.08 native amino acid sequence
MKRKNILKFVSLLGIGSFVMLAAASCTQASTPTPTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNSQNSAPLTDVSWIYTLSGTGAKYTLTFDYYGPQTGYLYFPYKLAKTSDKVGLQYKLNQADSVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLATSWSTYIRQFTGLTGNGVQTSDLVYLIGLIGGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNI

SEQ ID NO: 15 - DNA, 2103 bp, synthetic construct
  source 1..2103: mol_type = other DNA; note = Recombinant VlhA 4.08 protein DNA sequence
atgaaagcgaccaaactggtgctgggcgcggtgattctgggcagcaccctgctggcgggctgcagcagcaaccatggcattcgcggcagcattcgcacccaggcgagcaccccgaccccgaccccgaccccgaccccgaccccgaccccgaccccgaacccggaaccgaaaccgaacccgggcggcggcgataccaacaacccgccgagcggcggcatgaacggcgtggataccaacccgggcggcggccagggcatgatgaacgcgaccaaccaggaactggtgaacgcgaaaaaagcgctgagcgatctgattggcggcgaaagcaaaaccgtggaactgtatgcggattatgcgaaaattaaagcggatctgaccagcgcgtatgcggtggcgaaaaccaccagcgatagcagcaccagcaccctggatcaggtgaaaaccgcgaccagcaccctgcagaccgcgattaacaccgcggcgagcgataaagaaaaatttgatcagcagaacagccagctgctgatggcgtataaagtgctgaaagataccctgaacaaaaaagaagcgattgtgatgagcctgaaccaggaaaaatatagcgcgattctgagcgaaattaacgcggcgagcagcaccgcggaagaaattgtgaaacagaccctgaacccggtgaacggcaacctgccggtggtggcgaccctgaacgcggaaaacaccaaaattctggaagcgattaaagaagaaaaaattaacagcgaaaaaagcaacgcggatctgtttgcgaactatcagctgtataaactggatcgcaccaaactgatgagcgaaggcagcaacaacaccaaacagccgggcaactatagctttgtggcgtatgcgagcgatattgcgagcccgaactggaactttgcgcagcgcaccgtgtggaccgcggatagccgcacctggaccagcccgctgccgaacaacagccagaacagcgcgccgctgaccgatgtgagctggatttataccctgagcggcaccggcgcgaaatataccctgacctttgattattatggcccgcagaccggctatctgtattttccgtataaactggcgaaaaccagcgataaagtgggcctgcagtataaactgaaccaggcggatagcgtggcgattcagtttagcgaagcggcggcgaccgcgagcgcgccggcggaaaccgatggccgccaggaaagcgcggaaaccaccaccgcgaacgaaaaagtgaacccgatgccgagcgtgaacaccattaacgtggcgaaagtgaccctgagcaacctgaaatttggcagcaacaccattgaatttagcgtgccgatggatcaggataacatgaacaaagtggcgccgatgattggcaacatttatattaccagcagcaacgatgaagcgaacaaaaaacagatttatgatagcatttttggcaacaccagcagccagaccgcgagccagaccagcgtgagcgtggatctgctgaaaggctatagcctggcgaccagctggagcacctatattcgccagtttaccggcctgaccggcaacggcgtgcagaccagcgatctggtgtatctgattggcctgattggcggcagccagaaccgcaccgtggcgagcggcccgatgaacattaaaaacattccgaacatgaacggcgcgacccgcacctttaccatttatgcgaacgcgccggtgaacggcaactatcatattagcggcgcgtatctgcagggcgcgaaccaggcgcgcagcctgaaatttagcaccggcaccagcggcagcaacaacgaagtgaccgtgaccggcctgaaacagaaaaactggaccaccctgggccattttgataccaaaatgaccagcaccaccgtgagcggcaccggcaccccgatgaaacgcaccctgaccctgaaccagggcctgaacaaaattgtggtgagcggcgtgaacaacggcgataccccgtttattggcaacctgacctttaccctggaaagcaaccagaccattagcgaacaggtggaaccgagcagcgcgaccgcggaaaaaaacattgaaaacctgtattttcagagc

SEQ ID NO: 16 - protein, 701 aa, synthetic construct
  source 1..701: note = Recombinant VlhA 4.08 amino acid sequence
MKATKLVLGAVILGSTLLAGCSSNHGIRGSIRTQASTPTPTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNSQNSAPLTDVSWIYTLSGTGAKYTLTFDYYGPQTGYLYFPYKLAKTSDKVGLQYKLNQADSVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLATSWSTYIRQFTGLTGNGVQTSDLVYLIGLIGGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNIENLYFQS

SEQ ID NO: 17 - DNA, 1935 bp, Mycoplasma gallisepticum
  source 1..1935: mol_type = genomic DNA; note = vlhA 5.05 native DNA sequence
gtgaagagaaaaaacatattaaagtttgttagtttattaggtattggttcgtttgtgatgttagcagctgctagttgtactacaccaacacctaatccaaatcctcctagtggtggtatgaatggtggagatactaatccaggtgatggacaaggtatgatgaatgctgcatctcaagaattagctgctgcaagaatggggttaactactgtatttgattctaaagctaagaatcttggattgtatgttgactacaaaaaaacacaggacactctaacaaaagcatatgatgctgctaagactgttcttgataattcatcatcaacaacacaaaatcttaatgaggctaagactagattagaaactgcaattagaacagctgctactagtaaacaaacctttgatgaacaacatgccgagttagtaaaagtttatgaagaattaaaaacgactttaagtaatgaaactgctactttagttccatatgcagctgcacaatatgctggaattaagatgcatttaagtgggttatatgatgcaggtaaagcaattactactaaaacgttagaaccagttgagggagatccacttacagcatctgctgtaatgatggctaatactaagattgttgaagcaattaaagatgaagtattaaatcctcaaaaagaaaacgcaacaaaactagctgatagttttgttaagcaagtattagtaaaagaaaaaattacaggagttgaagaagctcataataaatcacaacctgctaactacagttttgtaggttacagcgttgatataactggaactgctaatggacaaacaagtattcctaactgaaattatgctcagagaaccattttcacaaacggtgatgaacctagaagtgtttctaatactcctgttgatggtcaaactatggcacaacctttatctaatgtgtcttgaatttatagtttagctggtaccggtgctaaatatactttagagtttacttactatggaccatcaacaggttatttatatttcccttataagttagttaatactagtgatcaagtgaaactaggtctagaatataaattaaatgatgcgactaaaccaagtgcgatcacttttggtagtgagcaaacaatgaatggtaaaactccaactgttaatgatattaatgtagctaaagttactttagctaacttaaattttggttcaaacaaaattgagtttagtgttccagtagaaaaagtaagtccgatgatcggtaatatgtatctttcttctagtcctaataactgaaataagatctatgatgatatttttgggaatagtgttacaactgaaaataatagaacaataatttcagttgatgctttaaacggttatagtttagcttcagattgatcaacatttatagctgaatatagtggcacaggtctaacattagataatcaaaatgtttcggaccaaaaatattatttaattgggtatgttggtggaactagttctcgtaatgatatgatgattccaacgaacaatgttcaaaaattcccattagcaagcaacataagtaatagaaactacatattttatgtaaatgctccaaaagcaggtgcttattatattaaaggagtgttcgcttcacgagatcctagagatcttaaattcagtactggagatatgtcttctaataatgtaactataagacaattgtctacagaaaatcttactacattaagaacctttgacacctctgcaacaacaggagctactcgagatacaactgtttctgatagaaagacattaactctagtagagggattaaataagatagtagttagtggagctactgaaaatacgggtaatgctccaaattttggatatttaaaatttattcttaatgaaacccaacctgaaactactaatgattcaaatccatcttag

SEQ ID NO: 18 - protein, 644 aa, Mycoplasma gallisepticum
  source 1..644: note = vlhA 5.05 native amino acid sequence
MKRKNILKFVSLLGIGSFVMLAAASCTTPTPNPNPPSGGMNGGDTNPGDGQGMMNAASQELAAARMGLTTVFDSKAKNLGLYVDYKKTQDTLTKAYDAAKTVLDNSSSTTQNLNEAKTRLETAIRTAATSKQTFDEQHAELVKVYEELKTTLSNETATLVPYAAAQYAGIKMHLSGLYDAGKAITTKTLEPVEGDPLTASAVMMANTKIVEAIKDEVLNPQKENATKLADSFVKQVLVKEKITGVEEAHNKSQPANYSFVGYSVDITGTANGQTSIPNWNYAQRTIFTNGDEPRSVSNTPVDGQTMAQPLSNVSWIYSLAGTGAKYTLEFTYYGPSTGYLYFPYKLVNTSDQVKLGLEYKLNDATKPSAITFGSEQTMNGKTPTVNDINVAKVTLANLNFGSNKIEFSVPVEKVSPMIGNMYLSSSPNNWNKIYDDIFGNSVTTENNRTIISVDALNGYSLASDWSTFIAEYSGTGLTLDNQNVSDQKYYLIGYVGGTSSRNDMMIPTNNVQKFPLASNISNRNYIFYVNAPKAGAYYIKGVFASRDPRDLKFSTGDMSSNNVTIRQLSTENLTTLRTFDTSATTGATRDTTVSDRKTLTLVEGLNKIVVSGATENTGNAPNFGYLKFILNETQPETTNDSNPS

SEQ ID NO: 19 - DNA, 1971 bp, Mycoplasma gallisepticum
  source 1..1971: mol_type = genomic DNA; note = Recombinant VlhA 5.05 nucleotide sequence
atgaaagcgaccaaactggtgctgggcgcggtgattctgggcagcaccctgctggcgggctgcagcagcaaccatggcattcgcggcagcattcgcaccaccccgaccccgaacccgaacccgccgagcggcggcatgaacggcggcgataccaacccgggcgatggccagggcatgatgaacgcggcgagccaggaactggcggcggcgcgcatgggcctgaccaccgtgtttgatagcaaagcgaaaaacctgggcctgtatgtggattataaaaaaacccaggataccctgaccaaagcgtatgatgcggcgaaaaccgtgctggataacagcagcagcaccacccagaacctgaacgaagcgaaaacccgcctggaaaccgcgattcgcaccgcggcgaccagcaaacagacctttgatgaacagcatgcggaactggtgaaagtgtatgaagaactgaaaaccaccctgagcaacgaaaccgcgaccctggtgccgtatgcggcggcgcagtatgcgggcattaaaatgcatctgagcggcctgtatgatgcgggcaaagcgattaccaccaaaaccctggaaccggtggaaggcgatccgctgaccgcgagcgcggtgatgatggcgaacaccaaaattgtggaagcgattaaagatgaagtgctgaacccgcagaaagaaaacgcgaccaaactggcggatagctttgtgaaacaggtgctggtgaaagaaaaaattaccggcgtggaagaagcgcataacaaaagccagccggcgaactatagctttgtgggctatagcgtggatattaccggcaccgcgaacggccagaccagcattccgaactggaactatgcgcagcgcaccatttttaccaacggcgatgaaccgcgcagcgtgagcaacaccccggtggatggccagaccatggcgcagccgctgagcaacgtgagctggatttatagcctggcgggcaccggcgcgaaatataccctggaatttacctattatggcccgagcaccggctatctgtattttccgtataaactggtgaacaccagcgatcaggtgaaactgggcctggaatataaactgaacgatgcgaccaaaccgagcgcgattacctttggcagcgaacagaccatgaacggcaaaaccccgaccgtgaacgatattaacgtggcgaaagtgaccctggcgaacctgaactttggcagcaacaaaattgaatttagcgtgccggtggaaaaagtgagcccgatgattggcaacatgtatctgagcagcagcccgaacaactggaacaaaatttatgatgatatttttggcaacagcgtgaccaccgaaaacaaccgcaccattattagcgtggatgcgctgaacggctatagcctggcgagcgattggagcacctttattgcggaatatagcggcaccggcctgaccctggataaccagaacgtgagcgatcagaaatattatctgattggctatgtgggcggcaccagcagccgcaacgatatgatgattccgaccaacaacgtgcagaaatttccgctggcgagcaacattagcaaccgcaactatattttttatgtgaacgcgccgaaagcgggcgcgtattatattaaaggcgtgtttgcgagccgcgatccgcgcgatctgaaatttagcaccggcgatatgagcagcaacaacgtgaccattcgccagctgagcaccgaaaacctgaccaccctgcgcacctttgataccagcgcgaccaccggcgcgacccgcgataccaccgtgagcgatcgcaaaaccctgaccctggtggaaggcctgaacaaaattgtggtgagcggcgcgaccgaaaacaccggcaacgcgccgaactttggctatctgaaatttattctgaacgaaacccagccggaaaccaccaacgatagcaacccgagcgaaaacctgtattttcagagc

SEQ ID NO: 20 - protein, 657 aa, synthetic construct
  source 1..657: note = Recombinant VlhA 5.05 protein amino acid sequence
MKATKLVLGAVILGSTLLAGCSSNHGIRGSIRTTPTPNPNPPSGGMNGGDTNPGDGQGMMNAASQELAAARMGLTTVFDSKAKNLGLYVDYKKTQDTLTKAYDAAKTVLDNSSSTTQNLNEAKTRLETAIRTAATSKQTFDEQHAELVKVYEELKTTLSNETATLVPYAAAQYAGIKMHLSGLYDAGKAITTKTLEPVEGDPLTASAVMMANTKIVEAIKDEVLNPQKENATKLADSFVKQVLVKEKITGVEEAHNKSQPANYSFVGYSVDITGTANGQTSIPNWNYAQRTIFTNGDEPRSVSNTPVDGQTMAQPLSNVSWIYSLAGTGAKYTLEFTYYGPSTGYLYFPYKLVNTSDQVKLGLEYKLNDATKPSAITFGSEQTMNGKTPTVNDINVAKVTLANLNFGSNKIEFSVPVEKVSPMIGNMYLSSSPNNWNKIYDDIFGNSVTTENNRTIISVDALNGYSLASDWSTFIAEYSGTGLTLDNQNVSDQKYYLIGYVGGTSSRNDMMIPTNNVQKFPLASNISNRNYIFYVNAPKAGAYYIKGVFASRDPRDLKFSTGDMSSNNVTIRQLSTENLTTLRTFDTSATTGATRDTTVSDRKTLTLVEGLNKIVVSGATENTGNAPNFGYLKFILNETQPETTNDSNPSENLYFQS

SEQ ID NO: 21 - DNA, 3189 bp, Mycoplasma gallisepticum
  source 1..3189: mol_type = genomic DNA; note = Mycoplasma gallisepticum crmA native DNA sequence
atgaatatttctaaaaaacttaaaagttatacattgataggtggattagctgtatttggagctcttggttctgcaagctttggctttaagcaatcagataagagtaacgataacacgcaattagttaatcaagcaagaacgctagatgctaattctgttagacttgcaggtcttggacaaaatggttcgttgttcaatacagttcttagagatgttgatgataactttataacagcagctaatggaacaattatcaaattagatagttttactaaaccattatatggtttagatctaagtgatgattttgctggatacaaagtaaaacaaatagtttcagattacacaactagcagaaatagatttgatcaaagacaaacaagagcatattatgctctgttggttaatgatgaagctaacgttcatttaaaaagaattaatactaactcaaatagaattggtaatagaaacaacaattctaagtttgtaattggtggtgttgataatccagctcacgtaattagatttactgatgatgggactaaatttaattttacaaagcaaactcaaggtgaaattgttaatgacttcattttagatgcgccaatcttacctaaagatttacacccagattgatataacttatacattcaaagaaagatcttaccaaatgacgtaaacactgcagttgttccttgaccagtaggtagagttagtggaacaaatgctgatgatgggatgtttgattttgggaatggtcaaataactaatacagatcctattgctcaaactaaaaccactactgataatcaaaatccttcaacttttaattcaggagcaatgcctggtgcaaacaatagatacgattctcaattgaatgtcaagcatagaattaaaacatctttccaattagatgaaaaatttgtttatccagaatgaactggttctgaagagaataaaaatattacaagattagctactggaagtttgccaagcaacgaaagatattgaattcttgacataccagggactccacaagttactttaaaagaagattcagttaacgtattttcaagactatacttaaactcagttaattctttatcattcattggtgatagtatttatatttttggtacttctgaattaccatcattatgatactattcattcccaactagattatctgatctaaccgctttgaatcaagttaaaacagatgatattgaagcttcaagcactgataacggtacaacaacaaacggaacaacgacaacaactgatacatctagtggttcaacaggtgctggaacaggaaatactactaacacttctcaaacagtttctaatcctactttaaatacttatcgtagttttggaattgatagtaaaccaacttctgcaaacaaaatagatgaaactaattgagcagatcctaatgttattgaagcaagaatatatgctgaatacagattaggtattcaaaatgaaattccaataactaatgcaggaaactttatccgaaacacaattggtggtgttggttttacttcaacaggttcaagagtagttttaagagcttcttataacggtgatcaacgtccaactggaaacttccaacctttcttatacgtatttggttatttaggataccaacaaactagaacaggaactttctgatacggaacatataaactattaaacaacagcccttacgacgtattagatgctgcaagagtaggtactgaaaccaatcaatttagaagaacttcattaacataccctgttatgggtggatatctaactgaagaaggtgctagaagtttctctaatactccatatataagagcacaaggtgacacaccagaaagccgaagcatcttccaatctggctattctgataatacttatgagtacattcaatcagttttaggatttgatggaattagaaataacttaaatgttggggttaaagcatcaagcttcttaaactcaaatagaccaaatccaaacggtctagaaatgattgctgcaacaacatacttaagatcacaaattggattagctagaacatctggattaccaaaccaacaaccattcggaacaactcaccaagttatttcagtatcacctggtgatcagttctcatcaattaagaatattagaacaatcttccctggtaaccagttatgatacttcttattcacaaatgaaaataataaatctagtgtttatacattaagattagctgactcaagtaaccctgatgcgtcaagctcattcagtccaacaagtttaattgacgttaatgaaattggtgtaatcttacctttattagacaattcattctatacagtaaatgctgctggtaatgttgcattgttctcatcaaaccctggttctcctggatcatatactgctgtaaatacatttaatcagaacttatctgatattgcttttgaaggttctggtgctaaatatacatctgatttctgaggaacaatccaattcaaacccgatgagtacttaattcaaaatgggttcactagtcaagtggctagaaacttcgttacaaaccaaagcttcttaaacagtttagttgacttcactcctgctaatgctggtactaactaccgtgtagtggttgatcctgatggtaatttaacaaaccaaaacctacctctaaaagttcagatccaatacttagatggtaagtattatgatgctaaattaaagaacaataatttagtaacattctcttataacaactttgctgctttaccttcatgagtagtgcctacagcaattggtagtacattaggtattcttgcaattatgatcatcttaggattagctatcggtattcctttaagagctcaaagaaaattacaagacaaagggttcaaaacaacattcaaaaaagttgataccttgactgctgctgttggttcagtttacaagaagattattacccaaactgctaacgttaagaaaaaacctgctgctttaggtgctggtaaatctggtgataagaaacctgctgctgctgctaaacctgctgctccagctaaaccatctgcaccaaaagctagctcaccagctaaaccaactgcgcctaaatctggtgcgcctacaaaaccaactgctcctaagccagctgctccaaaaccaaccgctcccaaagaataa

SEQ ID NO: 22 - protein, 1062 aa, Mycoplasma gallisepticum
  source 1..1062: note = Mycoplasma gallisepticum CrmA native aa sequence
MNISKKLKSYTLIGGLAVFGALGSASFGFKQSDKSNDNTQLVNQARTLDANSVRLAGLGQNGSLFNTVLRDVDDNFITAANGTIIKLDSFTKPLYGLDLSDDFAGYKVKQIVSDYTTSRNRFDQRQTRAYYALLVNDEANVHLKRINTNSNRIGNRNNNSKFVIGGVDNPAHVIRFTDDGTKFNFTKQTQGEIVNDFILDAPILPKDLHPDWYNLYIQRKILPNDVNTAVVPWPVGRVSGTNADDGMFDFGNGQITNTDPIAQTKTTTDNQNPSTFNSGAMPGANNRYDSQLNVKHRIKTSFQLDEKFVYPEWTGSEENKNITRLATGSLPSNERYWILDIPGTPQVTLKEDSVNVFSRLYLNSVNSLSFIGDSIYIFGTSELPSLWYYSFPTRLSDLTALNQVKTDDIEASSTDNGTTTNGTTTTTDTSSGSTGAGTGNTTNTSQTVSNPTLNTYRSFGIDSKPTSANKIDETNWADPNVIEARIYAEYRLGIQNEIPITNAGNFIRNTIGGVGFTSTGSRVVLRASYNGDQRPTGNFQPFLYVFGYLGYQQTRTGTFWYGTYKLLNNSPYDVLDAARVGTETNQFRRTSLTYPVMGGYLTEEGARSFSNTPYIRAQGDTPESRSIFQSGYSDNTYEYIQSVLGFDGIRNNLNVGVKASSFLNSNRPNPNGLEMIAATTYLRSQIGLARTSGLPNQQPFGTTHQVISVSPGDQFSSIKNIRTIFPGNQLWYFLFTNENNKSSVYTLRLADSSNPDASSSFSPTSLIDVNEIGVILPLLDNSFYTVNAAGNVALFSSNPGSPGSYTAVNTFNQNLSDIAFEGSGAKYTSDFWGTIQFKPDEYLIQNGFTSQVARNFVTNQSFLNSLVDFTPANAGTNYRVVVDPDGNLTNQNLPLKVQIQYLDGKYYDAKLKNNNLVTFSYNNFAALPSWVVPTAIGSTLGILAIMIILGLAIGIPLRAQRKLQDKGFKTTFKKVDTLTAAVGSVYKKIITQTANVKKKPAALGAGKSGDKKPAAAAKPAAPAKPSAPKASSPAKPTAPKSGAPTKPTAPKPAAPKPTAPKE

SEQ ID NO: 23 - DNA, 3207 bp, synthetic construct
  source 1..3207: mol_type = other DNA; note = Recombinant CrmA nucleotide sequence
atgaacattagcaaaaaactgaaaagctataccctgattggcggcctggcggtgtttggcgcgctgggcagcgcgagctttggctttaaacagagcgataaaagcaacgataacacccagctggtgaaccaggcgcgcaccctggatgcgaacagcgtgcgcctggcgggcctgggccagaacggcagcctgtttaacaccgtgctgcgcgatgtggatgataactttattaccgcggcgaacggcaccattattaaactggatagctttaccaaaccgctgtatggcctggatctgagcgatgattttgcgggctataaagtgaaacagattgtgagcgattataccaccagccgcaaccgctttgatcagcgccagacccgcgcgtattatgcgctgctggtgaacgatgaagcgaacgtgcatctgaaacgcattaacaccaacagcaaccgcattggcaaccgcaacaacaacagcaaatttgtgattggcggcgtggataacccggcgcatgtgattcgctttaccgatgatggcaccaaatttaactttaccaaacagacccagggcgaaattgtgaacgattttattctggatgcgccgattctgccgaaagatctgcatccggattggtataacctgtatattcagcgcaaaattctgccgaacgatgtgaacaccgcggtggtgccgtggccggtgggccgcgtgagcggcaccaacgcggatgatggcatgtttgattttggcaacggccagattaccaacaccgatccgattgcgcagaccaaaaccaccaccgataaccagaacccgagcacctttaacagcggcgcgatgccgggcgcgaacaaccgctatgatagccagctgaacgtgaaacatcgcattaaaaccagctttcagctggatgaaaaatttgtgtatccggaatggaccggcagcgaagaaaacaaaaacattacccgcctggcgaccggcagcctgccgagcaacgaacgctattggattctggatattccgggcaccccgcaggtgaccctgaaagaagatagcgtgaacgtgtttagccgcctgtatctgaacagcgtgaacagcctgagctttattggcgatagcatttatatttttggcaccagcgaactgccgagcctgtggtattatagctttccgacccgcctgagcgatctgaccgcgctgaaccaggtgaaaaccgatgatattgaagcgagcagcaccgataacggcaccaccaccaacggcaccaccaccaccaccgataccagcagcggcagcaccggcgcgggcaccggcaacaccaccaacaccagccagaccgtgagcaacccgaccctgaacacctatcgcagctttggcattgatagcaaaccgaccagcgcgaacaaaattgatgaaaccaactgggcggatccgaacgtgattgaagcgcgcatttatgcggaatatcgcctgggcattcagaacgaaattccgattaccaacgcgggcaactttattcgcaacaccattggcggcgtgggctttaccagcaccggcagccgcgtggtgctgcgcgcgagctataacggcgatcagcgcccgaccggcaactttcagccgtttctgtatgtgtttggctatctgggctatcagcagacccgcaccggcaccttttggtatggcacctataaactgctgaacaacagcccgtatgatgtgctggatgcggcgcgcgtgggcaccgaaaccaaccagtttcgccgcaccagcctgacctatccggtgatgggcggctatctgaccgaagaaggcgcgcgcagctttagcaacaccccgtatattcgcgcgcagggcgataccccggaaagccgcagcatttttcagagcggctatagcgataacacctatgaatatattcagagcgtgctgggctttgatggcattcgcaacaacctgaacgtgggcgtgaaagcgagcagctttctgaacagcaaccgcccgaacccgaacggcctggaaatgattgcggcgaccacctatctgcgcagccagattggcctggcgcgcaccagcggcctgccgaaccagcagccgtttggcaccacccatcaggtgattagcgtgagcccgggcgatcagtttagcagcattaaaaacattcgcaccatttttccgggcaaccagctgtggtattttctgtttaccaacgaaaacaacaaaagcagcgtgtataccctgcgcctggcggatagcagcaacccggatgcgagcagcagctttagcccgaccagcctgattgatgtgaacgaaattggcgtgattctgccgctgctggataacagcttttataccgtgaacgcggcgggcaacgtggcgctgtttagcagcaacccgggcagcccgggcagctataccgcggtgaacacctttaaccagaacctgagcgatattgcgtttgaaggcagcggcgcgaaatataccagcgatttttggggcaccattcagtttaaaccggatgaatatctgattcagaacggctttaccagccaggtggcgcgcaactttgtgaccaaccagagctttctgaacagcctggtggattttaccccggcgaacgcgggcaccaactatcgcgtggtggtggatccggatggcaacctgaccaaccagaacctgccgctgaaagtgcagattcagtatctggatggcaaatattatgatgcgaaactgaaaaacaacaacctggtgacctttagctataacaactttgcggcgctgccgagctgggtggtgccgaccgcgattggcagcaccctgggcattctggcgattatgattattctgggcctggcgattggcattccgctgcgcgcgcagcgcaaactgcaggataaaggctttaaaaccacctttaaaaaagtggataccctgaccgcggcggtgggcagcgtgtataaaaaaattattacccagaccgcgaacgtgaaaaaaaaaccggcggcgctgggcgcgggcaaaagcggcgataaaaaaccggcggcggcggcgaaaccggcggcgccggcgaaaccgagcgcgccgaaagcgagcagcccggcgaaaccgaccgcgccgaaaagcggcgcgccgaccaaaccgaccgcgccgaaaccggcggcgccgaaaccgaccgcgccgaaagaagaaaacctgtattttcagagc

SEQ ID NO: 24 - protein, 1069 aa, synthetic construct
  source 1..1069: note = Recombinant CrmA amino acid sequence
MNISKKLKSYTLIGGLAVFGALGSASFGFKQSDKSNDNTQLVNQARTLDANSVRLAGLGQNGSLFNTVLRDVDDNFITAANGTIIKLDSFTKPLYGLDLSDDFAGYKVKQIVSDYTTSRNRFDQRQTRAYYALLVNDEANVHLKRINTNSNRIGNRNNNSKFVIGGVDNPAHVIRFTDDGTKFNFTKQTQGEIVNDFILDAPILPKDLHPDWYNLYIQRKILPNDVNTAVVPWPVGRVSGTNADDGMFDFGNGQITNTDPIAQTKTTTDNQNPSTFNSGAMPGANNRYDSQLNVKHRIKTSFQLDEKFVYPEWTGSEENKNITRLATGSLPSNERYWILDIPGTPQVTLKEDSVNVFSRLYLNSVNSLSFIGDSIYIFGTSELPSLWYYSFPTRLSDLTALNQVKTDDIEASSTDNGTTTNGTTTTTDTSSGSTGAGTGNTTNTSQTVSNPTLNTYRSFGIDSKPTSANKIDETNWADPNVIEARIYAEYRLGIQNEIPITNAGNFIRNTIGGVGFTSTGSRVVLRASYNGDQRPTGNFQPFLYVFGYLGYQQTRTGTFWYGTYKLLNNSPYDVLDAARVGTETNQFRRTSLTYPVMGGYLTEEGARSFSNTPYIRAQGDTPESRSIFQSGYSDNTYEYIQSVLGFDGIRNNLNVGVKASSFLNSNRPNPNGLEMIAATTYLRSQIGLARTSGLPNQQPFGTTHQVISVSPGDQFSSIKNIRTIFPGNQLWYFLFTNENNKSSVYTLRLADSSNPDASSSFSPTSLIDVNEIGVILPLLDNSFYTVNAAGNVALFSSNPGSPGSYTAVNTFNQNLSDIAFEGSGAKYTSDFWGTIQFKPDEYLIQNGFTSQVARNFVTNQSFLNSLVDFTPANAGTNYRVVVDPDGNLTNQNLPLKVQIQYLDGKYYDAKLKNNNLVTFSYNNFAALPSWVVPTAIGSTLGILAIMIILGLAIGIPLRAQRKLQDKGFKTTFKKVDTLTAAVGSVYKKIITQTANVKKKPAALGAGKSGDKKPAAAAKPAAPAKPSAPKASSPAKPTAPKSGAPTKPTAPKPAAPKPTAPKEENLYFQS

SEQ ID NO: 25 - DNA, 3369 bp, Mycoplasma gallisepticum
  source 1..3369: mol_type = genomic DNA; note = Mycoplasma gallisepticum GapA native sequence
gtgaaaaaacttatttttaaattatcagtcggaataactcctcttgccttaatcggtttaggtagttttggattagcagtttctggagctaagccaaataaccttaaacctgttaaccaagttggggaaatgaattcacaaggccaatctaatcttttagagaaagctcgtagatgaagaaactctaacttcacatcactttcaattgacggtaccaacccaggtgcattagttttaactggatcaaaatcaattagccggattgatttgtatggtaacgtgatttgaacgtttgatccaggtaacacaaacgatctaactggtaaggttggattttatgatgctaacaatagattgactgcattttctggagacgttccttttaatgtaagtgatctaagctctaaaacagttgtagaagctactcaagatcaagaagatcctaatgttttctacttattattaattccagatgcagcggttcaacaagaacaaaagactaaagatcaagtgtttgaaaactactttatgtctgatgcacctgctactggtgatactagcgctgaaggttctgcaactcctgctggtggtggttcaagtagtagtgctgctggaggaggtgctgttgctcctgctgctgcgagttcgactgctagacttgttgaagaagggaatagtgctggtatgggaacgatgactcctactgcttctacttctgaaacagttatagattataatagcgatcaaaataaaattcctaaacctaaaacactattagacagtagcgaaagttctgaaagtatcaatggtggaagaacatatgcgaacattaacactcagaataatttacaaggtgttattgtaaaagttaacgaaaatttatttaattcggaaaatccctttgcagtagaaaatatggcgttcattaagccgaaggatatggttgataattatccttctacttgaacacaaggttctgctaacggtaaaatgactaacgttcttcaattctacaaacatgataatcctaatgctgttaacaatagattctatagagcaaaatactatcctaaacgtttagaaactcaaacaactactcctctaattgatagttctttctctccatatgagcatccagaatgatatgaagataatcaatttgtaatgccgtgaatgcagtacataacaaatttaggtggtttatatgctaaagatggaatggtgtacctatttggtggtaacggtacatgagttaacaacgaaagtgcattaagtattggtgttttcagaactaaatttgaaaacagaactgctgaagctccaggaaacactaaaactgttggttatccatacggtattttattatcagcgatttcttttgatgctactagaaatggattagcgcttgctcctgcaagtcttggtcaagatgttggttatcactttgttcctcgtcttgcagtgggtggtgtaagttcacctagaggagctaacggtaatattttcttaggttcagctattacttgaggaacaaacggtggtaatttcttagatactaaatgacacagtcctgctgtcattgaagatgcacctactacttttgtgactgttaatagtagtggtgcgcttcagaatagtggaaatccacaaccaacttctactccgatgcctaatagtaacggtaatgaaagcatcccttatagatgaacgaattcttatgattacaactctgtaagatttgcagctctaattagtaagccagctggtggaaacacaaaacaagttgaatcattatttacaaccgctttaaaattagatacattaaattctttaccaaataaatttactcaagaaaataatatcttctttagttatgctatgttagatggtcgtcaatgaagtttaggtacacgaaaagacagcgcatgattaacaactaatactattaataacttcacttataatactcaacaacaattagcgtctacagtagcaggagaaaacgctaatccaagaaatatcttaaacgctttaacaactgcaaaagggtttgatcgaagagatattggtaatgtagtatatacttattctaataatactaataagtttacttattactatcaagttggtggcgcgattacaacttgaccagaagttcaagtaaattacaaaacttcggctaatattacttactacaatttaactagaactgattttggaagtactactcctgcaactcaagatgcaaataccgtatcatctaaattaaacggcgcttacttatcatcaactggcgatcaacaaggatgatacaacggttcaatctatgttaaaaaagcgagctttacaccaagtagccaaggttatacttgacaagatttcaaaggtttaacaactacagcaagtaacgcagttatttctaactgaacaaaagctggatacagtattagaccagatgatgatacagtattcaacgtttctaagattccttttgaaaaagaaatcaccgctgctgttaatgtaagatcattagatagttactatgtacaattaaatggtgaaacttcagttaatactgtagctagagtaagtcctgattctagcgctttagccctaaaccctaacagaattactaacccattgatgaatagagataacgtaatcggtcaaggtgcgttcattagtagaaatgatattccatcatcattctttgaaaacaaaattaatgatattgtaactacagaagctgatggtaaagaagtattagatagtaaatacattaattcgatctatagatatactccacctcaaaacaatcctgatattagattaagattattagtaattgatcgttctagagcaaccaatgacttcattaagttattacctcaagtattagttgatggcgaatacgttgctgttccacaagctaatagtgtgtttgtgtctgaccaagaatttactggttttgatgcgcttccaggttatgtattaccagtagcgatctcgattccgatcatcataattgccttggcattagctttaggtctaggtattggtattccaatgtctcaaaaccgtaagatgttgaaacaaggatttgcgatttcaaacaaaaaagttgatattctgacaacagccgttggtagtgtgttcaaacaaattattaatcgaacatctgtgacaaatattaagaagactccacaaatgcttcaagccaacaagaaagatggagcatcttcaccaagcaagccatcagctccagctgctaagaaaccagcaggaccaactaaaccatctgctccaggggcaaaaccaacagcaccagctaaaccaaaagctccagcaccaactaagaaaattgaataa

SEQ ID NO: 26 - protein, 1122 aa, Mycoplasma gallisepticum
  source 1..1122: note = Mycoplasma gallisepticum native GapA sequence
MKKLIFKLSVGITPLALIGLGSFGLAVSGAKPNNLKPVNQVGEMNSQGQSNLLEKARRWRNSNFTSLSIDGTNPGALVLTGSKSISRIDLYGNVIWTFDPGNTNDLTGKVGFYDANNRLTAFSGDVPFNVSDLSSKTVVEATQDQEDPNVFYLLLIPDAAVQQEQKTKDQVFENYFMSDAPATGDTSAEGSATPAGGGSSSSAAGGGAVAPAAASSTARLVEEGNSAGMGTMTPTASTSETVIDYNSDQNKIPKPKTLLDSSESSESINGGRTYANINTQNNLQGVIVKVNENLFNSENPFAVENMAFIKPKDMVDNYPSTWTQGSANGKMTNVLQFYKHDNPNAVNNRFYRAKYYPKRLETQTTTPLIDSSFSPYEHPEWYEDNQFVMPWMQYITNLGGLYAKDGMVYLFGGNGTWVNNESALSIGVFRTKFENRTAEAPGNTKTVGYPYGILLSAISFDATRNGLALAPASLGQDVGYHFVPRLAVGGVSSPRGANGNIFLGSAITWGTNGGNFLDTKWHSPAVIEDAPTTFVTVNSSGALQNSGNPQPTSTPMPNSNGNESIPYRWTNSYDYNSVRFAALISKPAGGNTKQVESLFTTALKLDTLNSLPNKFTQENNIFFSYAMLDGRQWSLGTRKDSAWLTTNTINNFTYNTQQQLASTVAGENANPRNILNALTTAKGFDRRDIGNVVYTYSNNTNKFTYYYQVGGAITTWPEVQVNYKTSANITYYNLTRTDFGSTTPATQDANTVSSKLNGAYLSSTGDQQGWYNGSIYVKKASFTPSSQGYTWQDFKGLTTTASNAVISNWTKAGYSIRPDDDTVFNVSKIPFEKEITAAVNVRSLDSYYVQLNGETSVNTVARVSPDSSALALNPNRITNPLMNRDNVIGQGAFISRNDIPSSFFENKINDIVTTEADGKEVLDSKYINSIYRYTPPQNNPDIRLRLLVIDRSRATNDFIKLLPQVLVDGEYVAVPQANSVFVSDQEFTGFDALPGYVLPVAISIPIIIIALALALGLGIGIPMSQNRKMLKQGFAISNKKVDILTTAVGSVFKQIINRTSVTNIKKTPQMLQANKKDGASSPSKPSAPAAKKPAGPTKPSAPGAKPTAPAKPKAPAPTKKIE

SEQ ID NO: 27 - DNA, 3387 bp, synthetic construct
  source 1..3387: mol_type = other DNA; note = Recombinant GapA nucleotide sequence
atgaaaaaactgatttttaaactgagcgtgggcattaccccgctggcgctgattggcctgggcagctttggcctggcggtgagcggcgcgaaaccgaacaacctgaaaccggtgaaccaggtgggcgaaatgaacagccagggccagagcaacctgctggaaaaagcgcgccgctggcgcaacagcaactttaccagcctgagcattgatggcaccaacccgggcgcgctggtgctgaccggcagcaaaagcattagccgcattgatctgtatggcaacgtgatttggacctttgatccgggcaacaccaacgatctgaccggcaaagtgggcttttatgatgcgaacaaccgcctgaccgcgtttagcggcgatgtgccgtttaacgtgagcgatctgagcagcaaaaccgtggtggaagcgacccaggatcaggaagatccgaacgtgttttatctgctgctgattccggatgcggcggtgcagcaggaacagaaaaccaaagatcaggtgtttgaaaactattttatgagcgatgcgccggcgaccggcgataccagcgcggaaggcagcgcgaccccggcgggcggcggcagcagcagcagcgcggcgggcggcggcgcggtggcgccggcggcggcgagcagcaccgcgcgcctggtggaagaaggcaacagcgcgggcatgggcaccatgaccccgaccgcgagcaccagcgaaaccgtgattgattataacagcgatcagaacaaaattccgaaaccgaaaaccctgctggatagcagcgaaagcagcgaaagcattaacggcggccgcacctatgcgaacattaacacccagaacaacctgcagggcgtgattgtgaaagtgaacgaaaacctgtttaacagcgaaaacccgtttgcggtggaaaacatggcgtttattaaaccgaaagatatggtggataactatccgagcacctggacccagggcagcgcgaacggcaaaatgaccaacgtgctgcagttttataaacatgataacccgaacgcggtgaacaaccgcttttatcgcgcgaaatattatccgaaacgcctggaaacccagaccaccaccccgctgattgatagcagctttagcccgtatgaacatccggaatggtatgaagataaccagtttgtgatgccgtggatgcagtatattaccaacctgggcggcctgtatgcgaaagatggcatggtgtatctgtttggcggcaacggcacctgggtgaacaacgaaagcgcgctgagcattggcgtgtttcgcaccaaatttgaaaaccgcaccgcggaagcgccgggcaacaccaaaaccgtgggctatccgtatggcattctgctgagcgcgattagctttgatgcgacccgcaacggcctggcgctggcgccggcgagcctgggccaggatgtgggctatcattttgtgccgcgcctggcggtgggcggcgtgagcagcccgcgcggcgcgaacggcaacatttttctgggcagcgcgattacctggggcaccaacggcggcaactttctggataccaaatggcatagcccggcggtgattgaagatgcgccgaccacctttgtgaccgtgaacagcagcggcgcgctgcagaacagcggcaacccgcagccgaccagcaccccgatgccgaacagcaacggcaacgaaagcattccgtatcgctggaccaacagctatgattataacagcgtgcgctttgcggcgctgattagcaaaccggcgggcggcaacaccaaacaggtggaaagcctgtttaccaccgcgctgaaactggataccctgaacagcctgccgaacaaatttacccaggaaaacaacattttttttagctatgcgatgctggatggccgccagtggagcctgggcacccgcaaagatagcgcgtggctgaccaccaacaccattaacaactttacctataacacccagcagcagctggcgagcaccgtggcgggcgaaaacgcgaacccgcgcaacattctgaacgcgctgaccaccgcgaaaggctttgatcgccgcgatattggcaacgtggtgtatacctatagcaacaacaccaacaaatttacctattattatcaggtgggcggcgcgattaccacctggccggaagtgcaggtgaactataaaaccagcgcgaacattacctattataacctgacccgcaccgattttggcagcaccaccccggcgacccaggatgcgaacaccgtgagcagcaaactgaacggcgcgtatctgagcagcaccggcgatcagcagggctggtataacggcagcatttatgtgaaaaaagcgagctttaccccgagcagccagggctatacctggcaggattttaaaggcctgaccaccaccgcgagcaacgcggtgattagcaactggaccaaagcgggctatagcattcgcccggatgatgataccgtgtttaacgtgagcaaaattccgtttgaaaaagaaattaccgcggcggtgaacgtgcgcagcctggatagctattatgtgcagctgaacggcgaaaccagcgtgaacaccgtggcgcgcgtgagcccggatagcagcgcgctggcgctgaacccgaaccgcattaccaacccgctgatgaaccgcgataacgtgattggccagggcgcgtttattagccgcaacgatattccgagcagcttttttgaaaacaaaattaacgatattgtgaccaccgaagcggatggcaaagaagtgctggatagcaaatatattaacagcatttatcgctataccccgccgcagaacaacccggatattcgcctgcgcctgctggtgattgatcgcagccgcgcgaccaacgattttattaaactgctgccgcaggtgctggtggatggcgaatatgtggcggtgccgcaggcgaacagcgtgtttgtgagcgatcaggaatttaccggctttgatgcgctgccgggctatgtgctgccggtggcgattagcattccgattattattattgcgctggcgctggcgctgggcctgggcattggcattccgatgagccagaaccgcaaaatgctgaaacagggctttgcgattagcaacaaaaaagtggatattctgaccaccgcggtgggcagcgtgtttaaacagattattaaccgcaccagcgtgaccaacattaaaaaaaccccgcagatgctgcaggcgaacaaaaaagatggcgcgagcagcccgagcaaaccgagcgcgccggcggcgaaaaaaccggcgggcccgaccaaaccgagcgcgccgggcgcgaaaccgaccgcgccggcgaaaccgaaagcgccggcgccgaccaaaaaaattgaagaaaacctgtattttcagagc

SEQ ID NO: 28 - protein, 1129 aa, synthetic construct
  source 1..1129: note = Recombinant GapA amino acid sequence
MKKLIFKLSVGITPLALIGLGSFGLAVSGAKPNNLKPVNQVGEMNSQGQSNLLEKARRWRNSNFTSLSIDGTNPGALVLTGSKSISRIDLYGNVIWTFDPGNTNDLTGKVGFYDANNRLTAFSGDVPFNVSDLSSKTVVEATQDQEDPNVFYLLLIPDAAVQQEQKTKDQVFENYFMSDAPATGDTSAEGSATPAGGGSSSSAAGGGAVAPAAASSTARLVEEGNSAGMGTMTPTASTSETVIDYNSDQNKIPKPKTLLDSSESSESINGGRTYANINTQNNLQGVIVKVNENLFNSENPFAVENMAFIKPKDMVDNYPSTWTQGSANGKMTNVLQFYKHDNPNAVNNRFYRAKYYPKRLETQTTTPLIDSSFSPYEHPEWYEDNQFVMPWMQYITNLGGLYAKDGMVYLFGGNGTWVNNESALSIGVFRTKFENRTAEAPGNTKTVGYPYGILLSAISFDATRNGLALAPASLGQDVGYHFVPRLAVGGVSSPRGANGNIFLGSAITWGTNGGNFLDTKWHSPAVIEDAPTTFVTVNSSGALQNSGNPQPTSTPMPNSNGNESIPYRWTNSYDYNSVRFAALISKPAGGNTKQVESLFTTALKLDTLNSLPNKFTQENNIFFSYAMLDGRQWSLGTRKDSAWLTTNTINNFTYNTQQQLASTVAGENANPRNILNALTTAKGFDRRDIGNVVYTYSNNTNKFTYYYQVGGAITTWPEVQVNYKTSANITYYNLTRTDFGSTTPATQDANTVSSKLNGAYLSSTGDQQGWYNGSIYVKKASFTPSSQGYTWQDFKGLTTTASNAVISNWTKAGYSIRPDDDTVFNVSKIPFEKEITAAVNVRSLDSYYVQLNGETSVNTVARVSPDSSALALNPNRITNPLMNRDNVIGQGAFISRNDIPSSFFENKINDIVTTEADGKEVLDSKYINSIYRYTPPQNNPDIRLRLLVIDRSRATNDFIKLLPQVLVDGEYVAVPQANSVFVSDQEFTGFDALPGYVLPVAISIPIIIIALALALGLGIGIPMSQNRKMLKQGFAISNKKVDILTTAVGSVFKQIINRTSVTNIKKTPQMLQANKKDGASSPSKPSAPAAKKPAGPTKPSAPGAKPTAPAKPKAPAPTKKIEENLYFQS

SEQ ID NO: 29 - protein, 6 aa, synthetic construct
  source 1..6: note = His tag
HHHHHH

SEQ ID NO: 30 - DNA, 7213 bp, synthetic construct
  source 1..7213: mol_type = other DNA; note = Vector pET-28b(+) with vlhA 3.03
  misc_feature 5069..7056: note = inserted vlhA 3.03 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgaaagcgaccaaactggtgctgggcgcggtgattctgggcagcaccctgctggcgggctgcagcagcaacgatggcattcgcggcagcattcgcaccaccccgaccccgaacccgaacccgccgagcggcggcatgaacggcggcgataccaacccgggcgatggccagggcatgatgaacgcggcgagccaggaactggcggcggcgcgcatgggcctgaccaccgtgtttgatagcaaagcgaaaaacctgggcctgtatgtggattataaaaaaacccaggataccctgaccaaagcgtatgatgcggcgaaaaccgtgctggataacagcagcagcaccacccagaacctgaacgaagcgaaaacccgcctggaaaccgcgattcgcaccgcggcgaccagcaaacagacctttgatgaacagcatgcggaactggtgaaagtgtatgaagaactgaaaaccaccctgagcaacgaaaccgcgaccctggcgccgtatgcggatagccagtatgcgggcattaaaatgcatctgagcggcctgtatgatgcgggcaaagcgattaccaccaaaaccctggaaccggtggaaggcgatccgctgaccgcggatgtggtgatgatggcgaacaccaaaattgtggaagcgattaaagatgaagtgctgaacccgcagaaagaaaacgcgaccaaactggcggatagctttgtgaaacaggtgctggtgaaagaaaaaattaccggcgtggaagaagcgcataacaaagcgcagccggcgaactatagctttgtgggctatagcgtggatattaccggcaccgtgaccggccagaccagcattccgaactgggattatgcgcagcgcaccatttttaccaacggcgatgaaccgcgcagcattagcaacaccccggcggatggccagacgatggtgcagccgctgagcaacgtgagctggatttatagcctggcgggcaccggcgcgaaatataccctggaatttacctattatggcccgagcaccggctatctgtattttccgtataaactggtgaacaccagcgatcagatgaaactgggcctggaatataaactgaacgatgcgaccgaaccgagcgcgattacctttggcagcgaacagaccatgaacggcaaaaccccgaccgtgaacgatattaacgtggcgaaagtgaccctggcgaacctgaaatttggcagcaacaaaattgaatttagcgtgccggcggaaaaagtgagcccgatgattggcaacatgtatctgagcagcagcccgaacaactggaacaaaatttatgatgatatttttggcaacagcgtgaccaccgaaaacaaccgcaccattattagcgtggatgcgctgaacggctatagcctggcgagcgattggagcacctatattgcggaatatagcggcgcgggcctgaccctgaacgatcaggcgaaaccgaacgaaaaatattatctgattggctatgtgggcggcaccggcgcgcgcaacgatatgatggtgccgaaaaacaacgtgcagaaatttccgctggcgaacaacaccagcaaccgcaactatgtgttttatgtgaacgcgccgaaagcgggcgattattatattaaaggcgtgtttgcgagcggcgtgcatagcgatctgaaatttagcaccggcgatatgagcagcaacaacgtgaccgtgaaacagctgtttaccggcaacctgaccaccaccctgcgcacctttgataccagcgcgaccaccgaaagcacccgcgtgaccaccgatccgaccaacaaaaaaaccctgaccctggtggaaggcctgaacaaaattgtggtgagcggcaccaccgaaaacattggcgcgccgaactttggctatctggaatttattctgaacgaaacccagccggaaaccaccaacgtgagcaacccgagcgaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 31 - DNA, 7336 bp, synthetic construct
  source 1..7336: mol_type = other DNA; note = Vector pET-28b(+) with VlhA 3.06 inserted sequence
  misc_feature 5069..7179: note = Inserted vlhA 3.06 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagcgaccaaactggtgctgggcgcggtgattctgggcagcaccctgctggcgggctgcagcagcaatcatggcattcgcggcagcattcgcacccaggcgagcaccccgaccccgaccccgaccccgaccccgaccccgaccccgaccccgaacccggaaccgaaaccgaacccgggcggcggcgataccaacaacccgccgagcggcggcatgaacggcgtggataccaacccgggcggcggccagggcatgatgaacgcgaccaaccaggaactggtgaacgcgaaaaaagcgctgagcgatctgattggcggcgaaagcaaaaccgtggaactgtatgcggattatgcgaaaattaaagcggatctgaccagcgcgtatgcggtggcgaaaaccaccagcgatagcagcaccagcaccctggatcaggtgaaaaccgcgaccagcaccctgcagaccgcgattaacaccgcggcgagcgataaagaaaaatttgatcagcagaacagccagctgctgatggcgtataaagtgctgaaagataccctgaacaaaaaagaagcgattgtgatgagcctgaaccaggaaaaatatagcgcgattctgagcgaaattaacgcggcgagcagcaccgcggaagaaattgtgaaacagaccctgaacccggtgaacggcaacctgccggtggtggcgaccctgaacgcggaaaacaccaaaattctggaagcgattaaagaagaaaaaattaacagcgaaaaaagcaacgcggatctgtttgcgaactatcagctgtataaactggatcgcaccaaactgatgagcgaaggcagcaacaacaccaaacagccgggcaactatagctttgtggcgtatgcgagcgatattgcgagcccgaactggaactttgcgcagcgcaccgtgtggaccgcggatagccgcacctggaccagcccgctgccgaacaacctgcagaacagcgcgccgctgaccgatgtgagctggatttataccctgagcggcaccggcgcgaaatataccctgacctttgattattatggcccgcagaccggctatctgtattttccgtataaactggtgaaaaccagcgataaagtgggcctgcagtataaactgaaccaggcggatccggtggcgattcagtttagcgaagcggcggcgaccgcgagcgcgccggcggaaaccgatggccgccaggaaagcgcggaaaccaccaccgcgaacgaaaaagtgaacccgatgccgagcgtgaacaccattaacgtggcgaaagtgaccctgagcaacctgaaatttggcagcaacaccattgaatttagcgtgccgatggatcaggataacatgaacaaagtggcgccgatgattggcaacatttatattaccagcagcaacgatgaagcgaacaaaaaacagatttatgatagcatttttggcaacaccagcagccagaccgcgagccagaccagcgtgagcgtggatctgctgaaaggctatagcctgaccaccagctggagcacctatattcgccagtttaccggcctgaccggcaacggcgtgcagaccagcgatctggtgtatctgattggcctgattgatggcagccagaaccgcaccgtggcgagcggcccgatgaacattaaaaacattccgaacatgaacggcgcgacccgcacctttaccatttatgcgaacgcgccggtgaacggcaactatcatattagcggcgcgtatctgcagggcgcgaaccaggcgcgcagcctgaaatttagcaccggcaccagcggcagcaacaacgaagtgaccgtgaccggcctgaaacagaaaaactggaccaccctgggccattttgataccaaaatgaccagcaccaccgtgagcggcaccggcaccccgatgaaacgcaccctgaccctgaaccagggcctgaacaaaattgtggtgagcggcgtgaacaacggcgataccccgtttattggcaacctgacctttaccctggaaagcaaccagaccattagcgaacaggtggaaccgagcagcgcgaccgcggaaaaaaacattgaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 32 - DNA, 7273 bp, synthetic construct
  source 1..7273: mol_type = other DNA; note = Vector pET-28b(+) with inserted VlhA 4.07 sequence
  misc_feature 5069..7116: note = Inserted VlhA 4.08 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagcgaccaaactggtgctgggcgcggtgattctgggcagcaccctgctggcgggctgcagcagcaatcatggcattcgcggcagcattcgcaccagcgcgaccaccccgaccccgaacccgaccccgaaaccggatccgatgccgaacccgggcggcggcatgaacggcggcaacaccaaccgcggcaacggcggcggcatgatgggcgataacccgaacccgggcaacaccaccccggaacagcagctggcggcggcgcgcaaaaccctgaccgatctgctgggcaccgaaaacaccaacgtggcgttttatgcggattatgcgaaaattcagagcaccctgaacgcggcgtatatgaccgcgaaaaccgcgagcgaaaacaccagcgcgagcctggaaaacctgcgcagcgcgagcaccaccctgcaggcggcgattgataaagcggcgagcgataaacgcgcgtttgatagcgcgaaccagagcctggtgaccgcgtataacaacctgaaaaccaccctgcagtttaaaaccaccagcctggaagaactgagcgaaaacaaatatagcggcattaaaaaccatctgagcaaactgtttgataccggcagcgcgattaccgcgaaaaccctggatccgaccagcggcgaacgcccgaccctggaaaaagtgaacgaagcgaacaacagcattaaaatggcgattagcccggaaagcctgaaaaaatggaaagataacgcggataaatttaacgaatttgaaaaaaacccgctgagcaaagaaaaactgagcaccggcaccgatagcgcgcataaccaggaacagccggcgaactatagctttgtgggctatagcgtggatgtgaccggcaccaccaccgcgccggcgagcgaaaaagcgctgctgaactggaactatgcgcagcgcaccatttttaccagcggcgatcagccgaccaaaattgataccccggcggcgagcgatgaagcgccggcgcagccgctgagcaacgtgagctggatttatagcctggcgggcaacggcgcgaaatataccctggattttacctattatggcccgagcaccggctatctgtattttccgtataaactggtgaaaaacagcgatagcgtgggcctgcagtataaactgaacaacagcccgaccgaaaccgcgattgcgtttggcaacatgcagaacgcgaacggcccgaccgcgaccgtggatagcattaacattgcgaaagtgaccctgagcaacctgaactttggcgcgaacaaaattgaatttagcgtgccgagcgaaaaagtggcgccgatgattggcaacatgtatctgaccagcagcgcggatgaagcgaacaaacagaaaattgaaaacagcatttttggcaacagcgtgaccaccgaaaacaaccgcaccattattagcgtggatgcgctgagcggctatagcctggcgagcgattggagcacctttattggccgctatgcggatcagcagctgaccctggatggcaaaatgatgcgcgatcagaaatattatctgattggctatgtgggcggcaacaccggcgcgcgcaacgtgaccggcattaccgaaaaaaccaacgaacagcgcagcccggtggtgagcaccccgaaaaaacgcacctataccttttatgtgaacgcgccgaaagcgggcgcgtattatattaaaggcgtgtttgcgagcaaagtgcgccgcgatctgaaatttagcaccggcgatatgagcagcaacaacgtgaccattcagcagctgagcaccaacaacctgaccaccctgcgcacctttgataccagcgcgaccaccggcgcgacccgcgtgaccaccgtgagcgatcgcaaaaccctgaccctggtggaaggcctgaacaaaattgtggtgagcggcgcgaccgcggataacggcaacgcgccgaactttggctatctggaatttattctgaacgaaacccagccggaaaccaccaacgtgagcaacccgagcgaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 33 - DNA, 7336 bp, synthetic construct
  source 1..7336: mol_type = other DNA; note = Vector pET-28b(+) with VlhA 4.08 sequence
  misc_feature 5069..7179: note = Inserted VlhA 4.08 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagcgaccaaactggtgctgggcgcggtgattctgggcagcaccctgctggcgggctgcagcagcaatcatggcattcgcggcagcattcgcacccaggcgagcaccccgaccccgaccccgaccccgaccccgaccccgaccccgaccccgaacccggaaccgaaaccgaacccgggcggcggcgataccaacaacccgccgagcggcggcatgaacggcgtggataccaacccgggcggcggccagggcatgatgaacgcgaccaaccaggaactggtgaacgcgaaaaaagcgctgagcgatctgattggcggcgaaagcaaaaccgtggaactgtatgcggattatgcgaaaattaaagcggatctgaccagcgcgtatgcggtggcgaaaaccaccagcgatagcagcaccagcaccctggatcaggtgaaaaccgcgaccagcaccctgcagaccgcgattaacaccgcggcgagcgataaagaaaaatttgatcagcagaacagccagctgctgatggcgtataaagtgctgaaagataccctgaacaaaaaagaagcgattgtgatgagcctgaaccaggaaaaatatagcgcgattctgagcgaaattaacgcggcgagcagcaccgcggaagaaattgtgaaacagaccctgaacccggtgaacggcaacctgccggtggtggcgaccctgaacgcggaaaacaccaaaattctggaagcgattaaagaagaaaaaattaacagcgaaaaaagcaacgcggatctgtttgcgaactatcagctgtataaactggatcgcaccaaactgatgagcgaaggcagcaacaacaccaaacagccgggcaactatagctttgtggcgtatgcgagcgatattgcgagcccgaactggaactttgcgcagcgcaccgtgtggaccgcggatagccgcacctggaccagcccgctgccgaacaacagccagaacagcgcgccgctgaccgatgtgagctggatttataccctgagcggcaccggcgcgaaatataccctgacctttgattattatggcccgcagaccggctatctgtattttccgtataaactggcgaaaaccagcgataaagtgggcctgcagtataaactgaaccaggcggatagcgtggcgattcagtttagcgaagcggcggcgaccgcgagcgcgccggcggaaaccgatggccgccaggaaagcgcggaaaccaccaccgcgaacgaaaaagtgaacccgatgccgagcgtgaacaccattaacgtggcgaaagtgaccctgagcaacctgaaatttggcagcaacaccattgaatttagcgtgccgatggatcaggataacatgaacaaagtggcgccgatgattggcaacatttatattaccagcagcaacgatgaagcgaacaaaaaacagatttatgatagcatttttggcaacaccagcagccagaccgcgagccagaccagcgtgagcgtggatctgctgaaaggctatagcctggcgaccagctggagcacctatattcgccagtttaccggcctgaccggcaacggcgtgcagaccagcgatctggtgtatctgattggcctgattggcggcagccagaaccgcaccgtggcgagcggcccgatgaacattaaaaacattccgaacatgaacggcgcgacccgcacctttaccatttatgcgaacgcgccggtgaacggcaactatcatattagcggcgcgtatctgcagggcgcgaaccaggcgcgcagcctgaaatttagcaccggcaccagcggcagcaacaacgaagtgaccgtgaccggcctgaaacagaaaaactggaccaccctgggccattttgataccaaaatgaccagcaccaccgtgagcggcaccggcaccccgatgaaacgcaccctgaccctgaaccagggcctgaacaaaattgtggtgagcggcgtgaacaacggcgataccccgtttattggcaacctgacctttaccctggaaagcaaccagaccattagcgaacaggtggaaccgagcagcgcgaccgcggaaaaaaacattgaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 34 - DNA, 7204 bp, synthetic construct
  source 1..7204: mol_type = other DNA; note = Vector pET-28b(+) with vlhA 5.05 inserted sequence
  misc_feature 5069..7047: note = inserted VlhA 5.05 sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaagcgaccaaactggtgctgggcgcggtgattctgggcagcaccctgctggcgggctgcagcagcaatcatggcattcgcggcagcattcgcaccaccccgaccccgaacccgaacccgccgagcggcggcatgaacggcggcgataccaacccgggcgatggccagggcatgatgaacgcggcgagccaggaactggcggcggcgcgcatgggcctgaccaccgtgtttgatagcaaagcgaaaaacctgggcctgtatgtggattataaaaaaacccaggataccctgaccaaagcgtatgatgcggcgaaaaccgtgctggataacagcagcagcaccacccagaacctgaacgaagcgaaaacccgcctggaaaccgcgattcgcaccgcggcgaccagcaaacagacctttgatgaacagcatgcggaactggtgaaagtgtatgaagaactgaaaaccaccctgagcaacgaaaccgcgaccctggtgccgtatgcggcggcgcagtatgcgggcattaaaatgcatctgagcggcctgtatgatgcgggcaaagcgattaccaccaaaaccctggaaccggtggaaggcgatccgctgaccgcgagcgcggtgatgatggcgaacaccaaaattgtggaagcgattaaagatgaagtgctgaacccgcagaaagaaaacgcgaccaaactggcggatagctttgtgaaacaggtgctggtgaaagaaaaaattaccggcgtggaagaagcgcataacaaaagccagccggcgaactatagctttgtgggctatagcgtggatattaccggcaccgcgaacggccagaccagcattccgaactggaactatgcgcagcgcaccatttttaccaacggcgatgaaccgcgcagcgtgagcaacaccccggtggatggccagaccatggcgcagccgctgagcaacgtgagctggatttatagcctggcgggcaccggcgcgaaatataccctggaatttacctattatggcccgagcaccggctatctgtattttccgtataaactggtgaacaccagcgatcaggtgaaactgggcctggaatataaactgaacgatgcgaccaaaccgagcgcgattacctttggcagcgaacagaccatgaacggcaaaaccccgaccgtgaacgatattaacgtggcgaaagtgaccctggcgaacctgaactttggcagcaacaaaattgaatttagcgtgccggtggaaaaagtgagcccgatgattggcaacatgtatctgagcagcagcccgaacaactggaacaaaatttatgatgatatttttggcaacagcgtgaccaccgaaaacaaccgcaccattattagcgtggatgcgctgaacggctatagcctggcgagcgattggagcacctttattgcggaatatagcggcaccggcctgaccctggataaccagaacgtgagcgatcagaaatattatctgattggctatgtgggcggcaccagcagccgcaacgatatgatgattccgaccaacaacgtgcagaaatttccgctggcgagcaacattagcaaccgcaactatattttttatgtgaacgcgccgaaagcgggcgcgtattatattaaaggcgtgtttgcgagccgcgatccgcgcgatctgaaatttagcaccggcgatatgagcagcaacaacgtgaccattcgccagctgagcaccgaaaacctgaccaccctgcgcacctttgataccagcgcgaccaccggcgcgacccgcgataccaccgtgagcgatcgcaaaaccctgaccctggtggaaggcctgaacaaaattgtggtgagcggcgcgaccgaaaacaccggcaacgcgccgaactttggctatctgaaatttattctgaacgaaacccagccggaaaccaccaacgatagcaacccgagcgaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 35 - DNA, 8446 bp, synthetic construct
  source 1..8446: mol_type = other DNA; note = Vector pET-28b(+) with inserted CrmA sequence
  misc_feature 5069..8289: note = Inserted CrmA sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgaacattagcaaaaaactgaaaagctataccctgattggcggcctggcggtgtttggcgcgctgggcagcgcgagctttggctttaaacagagcgataaaagcaacgataacacccagctggtgaaccaggcgcgcaccctggatgcgaacagcgtgcgcctggcgggcctgggccagaacggcagcctgtttaacaccgtgctgcgcgatgtggatgataactttattaccgcggcgaacggcaccattattaaactggatagctttaccaaaccgctgtatggcctggatctgagcgatgattttgcgggctataaagtgaaacagattgtgagcgattataccaccagccgcaaccgctttgatcagcgccagacccgcgcgtattatgcgctgctggtgaacgatgaagcgaacgtgcatctgaaacgcattaacaccaacagcaaccgcattggcaaccgcaacaacaacagcaaatttgtgattggcggcgtggataacccggcgcatgtgattcgctttaccgatgatggcaccaaatttaactttaccaaacagacccagggcgaaattgtgaacgattttattctggatgcgccgattctgccgaaagatctgcatccggattggtataacctgtatattcagcgcaaaattctgccgaacgatgtgaacaccgcggtggtgccgtggccggtgggccgcgtgagcggcaccaacgcggatgatggcatgtttgattttggcaacggccagattaccaacaccgatccgattgcgcagaccaaaaccaccaccgataaccagaacccgagcacctttaacagcggcgcgatgccgggcgcgaacaaccgctatgatagccagctgaacgtgaaacatcgcattaaaaccagctttcagctggatgaaaaatttgtgtatccggaatggaccggcagcgaagaaaacaaaaacattacccgcctggcgaccggcagcctgccgagcaacgaacgctattggattctggatattccgggcaccccgcaggtgaccctgaaagaagatagcgtgaacgtgtttagccgcctgtatctgaacagcgtgaacagcctgagctttattggcgatagcatttatatttttggcaccagcgaactgccgagcctgtggtattatagctttccgacccgcctgagcgatctgaccgcgctgaaccaggtgaaaaccgatgatattgaagcgagcagcaccgataacggcaccaccaccaacggcaccaccaccaccaccgataccagcagcggcagcaccggcgcgggcaccggcaacaccaccaacaccagccagaccgtgagcaacccgaccctgaacacctatcgcagctttggcattgatagcaaaccgaccagcgcgaacaaaattgatgaaaccaactgggcggatccgaacgtgattgaagcgcgcatttatgcggaatatcgcctgggcattcagaacgaaattccgattaccaacgcgggcaactttattcgcaacaccattggcggcgtgggctttaccagcaccggcagccgcgtggtgctgcgcgcgagctataacggcgatcagcgcccgaccggcaactttcagccgtttctgtatgtgtttggctatctgggctatcagcagacccgcaccggcaccttttggtatggcacctataaactgctgaacaacagcccgtatgatgtgctggatgcggcgcgcgtgggcaccgaaaccaaccagtttcgccgcaccagcctgacctatccggtgatgggcggctatctgaccgaagaaggcgcgcgcagctttagcaacaccccgtatattcgcgcgcagggcgataccccggaaagccgcagcatttttcagagcggctatagcgataacacctatgaatatattcagagcgtgctgggctttgatggcattcgcaacaacctgaacgtgggcgtgaaagcgagcagctttctgaacagcaaccgcccgaacccgaacggcctggaaatgattgcggcgaccacctatctgcgcagccagattggcctggcgcgcaccagcggcctgccgaaccagcagccgtttggcaccacccatcaggtgattagcgtgagcccgggcgatcagtttagcagcattaaaaacattcgcaccatttttccgggcaaccagctgtggtattttctgtttaccaacgaaaacaacaaaagcagcgtgtataccctgcgcctggcggatagcagcaacccggatgcgagcagcagctttagcccgaccagcctgattgatgtgaacgaaattggcgtgattctgccgctgctggataacagcttttataccgtgaacgcggcgggcaacgtggcgctgtttagcagcaacccgggcagcccgggcagctataccgcggtgaacacctttaaccagaacctgagcgatattgcgtttgaaggcagcggcgcgaaatataccagcgatttttggggcaccattcagtttaaaccggatgaatatctgattcagaacggctttaccagccaggtggcgcgcaactttgtgaccaaccagagctttctgaacagcctggtggattttaccccggcgaacgcgggcaccaactatcgcgtggtggtggatccggatggcaacctgaccaaccagaacctgccgctgaaagtgcagattcagtatctggatggcaaatattatgatgcgaaactgaaaaacaacaacctggtgacctttagctataacaactttgcggcgctgccgagctgggtggtgccgaccgcgattggcagcaccctgggcattctggcgattatgattattctgggcctggcgattggcattccgctgcgcgcgcagcgcaaactgcaggataaaggctttaaaaccacctttaaaaaagtggataccctgaccgcggcggtgggcagcgtgtataaaaaaattattacccagaccgcgaacgtgaaaaaaaaaccggcggcgctgggcgcgggcaaaagcggcgataaaaaaccggcggcggcggcgaaaccggcggcgccggcgaaaccgagcgcgccgaaagcgagcagcccggcgaaaccgaccgcgccgaaaagcggcgcgccgaccaaaccgaccgcgccgaaaccggcggcgccgaaaccgaccgcgccgaaagaagaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 36 - DNA, 8626 bp, synthetic construct
  source 1..8626: mol_type = other DNA; note = Vector pET-28b(+) with inserted GapA sequence
  misc_feature 5069..8469: note = Inserted GapA sequence
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcatgaaaaaactgatttttaaactgagcgtgggcattaccccgctggcgctgattggcctgggcagctttggcctggcggtgagcggcgcgaaaccgaacaacctgaaaccggtgaaccaggtgggcgaaatgaacagccagggccagagcaacctgctggaaaaagcgcgccgctggcgcaacagcaactttaccagcctgagcattgatggcaccaacccgggcgcgctggtgctgaccggcagcaaaagcattagccgcattgatctgtatggcaacgtgatttggacctttgatccgggcaacaccaacgatctgaccggcaaagtgggcttttatgatgcgaacaaccgcctgaccgcgtttagcggcgatgtgccgtttaacgtgagcgatctgagcagcaaaaccgtggtggaagcgacccaggatcaggaagatccgaacgtgttttatctgctgctgattccggatgcggcggtgcagcaggaacagaaaaccaaagatcaggtgtttgaaaactattttatgagcgatgcgccggcgaccggcgataccagcgcggaaggcagcgcgaccccggcgggcggcggcagcagcagcagcgcggcgggcggcggcgcggtggcgccggcggcggcgagcagcaccgcgcgcctggtggaagaaggcaacagcgcgggcatgggcaccatgaccccgaccgcgagcaccagcgaaaccgtgattgattataacagcgatcagaacaaaattccgaaaccgaaaaccctgctggatagcagcgaaagcagcgaaagcattaacggcggccgcacctatgcgaacattaacacccagaacaacctgcagggcgtgattgtgaaagtgaacgaaaacctgtttaacagcgaaaacccgtttgcggtggaaaacatggcgtttattaaaccgaaagatatggtggataactatccgagcacctggacccagggcagcgcgaacggcaaaatgaccaacgtgctgcagttttataaacatgataacccgaacgcggtgaacaaccgcttttatcgcgcgaaatattatccgaaacgcctggaaacccagaccaccaccccgctgattgatagcagctttagcccgtatgaacatccggaatggtatgaagataaccagtttgtgatgccgtggatgcagtatattaccaacctgggcggcctgtatgcgaaagatggcatggtgtatctgtttggcggcaacggcacctgggtgaacaacgaaagcgcgctgagcattggcgtgtttcgcaccaaatttgaaaaccgcaccgcggaagcgccgggcaacaccaaaaccgtgggctatccgtatggcattctgctgagcgcgattagctttgatgcgacccgcaacggcctggcgctggcgccggcgagcctgggccaggatgtgggctatcattttgtgccgcgcctggcggtgggcggcgtgagcagcccgcgcggcgcgaacggcaacatttttctgggcagcgcgattacctggggcaccaacggcggcaactttctggataccaaatggcatagcccggcggtgattgaagatgcgccgaccacctttgtgaccgtgaacagcagcggcgcgctgcagaacagcggcaacccgcagccgaccagcaccccgatgccgaacagcaacggcaacgaaagcattccgtatcgctggaccaacagctatgattataacagcgtgcgctttgcggcgctgattagcaaaccggcgggcggcaacaccaaacaggtggaaagcctgtttaccaccgcgctgaaactggataccctgaacagcctgccgaacaaatttacccaggaaaacaacattttttttagctatgcgatgctggatggccgccagtggagcctgggcacccgcaaagatagcgcgtggctgaccaccaacaccattaacaactttacctataacacccagcagcagctggcgagcaccgtggcgggcgaaaacgcgaacccgcgcaacattctgaacgcgctgaccaccgcgaaaggctttgatcgccgcgatattggcaacgtggtgtatacctatagcaacaacaccaacaaatttacctattattatcaggtgggcggcgcgattaccacctggccggaagtgcaggtgaactataaaaccagcgcgaacattacctattataacctgacccgcaccgattttggcagcaccaccccggcgacccaggatgcgaacaccgtgagcagcaaactgaacggcgcgtatctgagcagcaccggcgatcagcagggctggtataacggcagcatttatgtgaaaaaagcgagctttaccccgagcagccagggctatacctggcaggattttaaaggcctgaccaccaccgcgagcaacgcggtgattagcaactggaccaaagcgggctatagcattcgcccggatgatgataccgtgtttaacgtgagcaaaattccgtttgaaaaagaaattaccgcggcggtgaacgtgcgcagcctggatagctattatgtgcagctgaacggcgaaaccagcgtgaacaccgtggcgcgcgtgagcccggatagcagcgcgctggcgctgaacccgaaccgcattaccaacccgctgatgaaccgcgataacgtgattggccagggcgcgtttattagccgcaacgatattccgagcagcttttttgaaaacaaaattaacgatattgtgaccaccgaagcggatggcaaagaagtgctggatagcaaatatattaacagcatttatcgctataccccgccgcagaacaacccggatattcgcctgcgcctgctggtgattgatcgcagccgcgcgaccaacgattttattaaactgctgccgcaggtgctggtggatggcgaatatgtggcggtgccgcaggcgaacagcgtgtttgtgagcgatcaggaatttaccggctttgatgcgctgccgggctatgtgctgccggtggcgattagcattccgattattattattgcgctggcgctggcgctgggcctgggcattggcattccgatgagccagaaccgcaaaatgctgaaacagggctttgcgattagcaacaaaaaagtggatattctgaccaccgcggtgggcagcgtgtttaaacagattattaaccgcaccagcgtgaccaacattaaaaaaaccccgcagatgctgcaggcgaacaaaaaagatggcgcgagcagcccgagcaaaccgagcgcgccggcggcgaaaaaaccggcgggcccgaccaaaccgagcgcgccgggcgcgaaaccgaccgcgccggcgaaaccgaaagcgccggcgccgaccaaaaaaattgaagaaaacctgtattttcagagcctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 37 - protein, 7 aa, synthetic construct
  source 1..7: note = recognition site of the tobacco etch virus (TEV) protease
ENLYFQS

SEQ ID NO: 38 - DNA, 2748 bp, synthetic construct
  source 1..2748: mol_type = other DNA; note = Recombinant crm-EC nucleotide sequence
atgagtttcggttttaagcagagcgataaaagtaatgataacacccagctggtgaatcaggcccgcaccctggatgcaaatagtgttcgcctggcaggtctgggccagaatggtagcctgtttaataccgtgctgcgtgatgtggatgataattttattaccgcagcaaatggtaccattattaagctggatagttttaccaaaccgctgtatggtctggatctgagcgatgattttgcaggctataaagtgaaacagattgttagcgattacaccaccagccgcaatcgctttgatcagcgtcagacccgtgcatattatgccctgctggtgaatgatgaagcaaatgttcatctgaaacgcattaataccaatagtaatcgcattggcaatcgtaataataatagtaaattcgtgatcggcggcgtggataatccggcacatgttattcgttttaccgatgatggtaccaaattcaattttaccaaacagacccagggcgaaattgtgaatgattttattctggatgccccgattctgccgaaagatctgcatccggattggtataatctgtatattcagcgcaaaattctgccgaatgatgtgaataccgcagtggttccgtggccggttggccgtgttagcggtaccaatgcagatgatggcatgtttgattttggcaatggccagattaccaataccgatccgattgcacagaccaaaaccaccaccgataatcagaatccgagcacctttaatagcggtgcaatgccgggcgccaataatcgctatgatagccagctgaatgttaaacatcgcattaagaccagctttcagctggatgaaaaatttgtttatccggaatggaccggcagtgaagaaaataagaatattacccgcctggcaaccggtagcctgccgagtaatgaacgttattggattctggatattccgggcaccccgcaggttaccctgaaagaagatagcgtgaatgtgtttagccgtctgtatctgaatagcgttaatagcctgagctttattggtgacagtatctatattttcggtaccagcgaactgccgagtctgtggtattatagttttccgacccgtctgagtgatctgaccgcactgaatcaggttaaaaccgatgatattgaagcaagtagtaccgataatggtaccaccaccaatggcaccaccaccaccacagataccagcagtggtagtaccggtgcaggtaccggtaataccaccaataccagtcagaccgtgagcaatccgaccctgaatacctatcgcagttttggtattgatagcaaaccgaccagtgcaaataagattgatgaaaccaattgggccgatccgaatgttattgaagcccgcatctatgccgaatatcgtctgggcattcagaatgaaattccgattaccaatgcaggtaattttattcgcaataccattggcggtgtgggctttaccagtaccggcagtcgcgtggtgctgcgtgcaagttataatggcgatcagcgtccgaccggcaattttcagccgtttctgtatgtttttggctatctgggttatcagcagacccgcaccggtaccttttggtatggcacctataaactgctgaataatagtccgtatgatgttctggatgcagcccgtgtgggtaccgaaaccaatcagtttcgccgtaccagcctgacctatccggttatgggtggctatctgaccgaagaaggtgcccgtagttttagtaataccccgtatattcgtgcacagggtgacaccccggaaagtcgtagtatttttcagagcggttatagcgataatacctatgaatatatccagagtgtgctgggctttgatggcattcgcaataatctgaatgtgggtgtgaaagccagtagttttctgaatagtaatcgtccgaatccgaatggtctggaaatgattgccgccaccacctatctgcgtagccagattggcctggcccgtaccagcggcctgccgaatcagcagccgtttggtaccacccatcaggttattagtgttagtccgggtgaccagtttagtagcattaagaatattcgtaccatctttccgggtaatcagctgtggtattttctgtttaccaatgaaaataacaagagcagcgtgtataccctgcgtctggccgatagcagtaatccggatgcaagtagtagttttagcccgaccagcctgattgatgtgaatgaaattggtgttattctgccgctgctggataatagtttttataccgtgaatgcagcaggtaatgttgcactgtttagcagcaatccgggcagtccgggtagctataccgccgtgaatacctttaatcagaatctgagtgatatcgcatttgaaggcagcggtgccaaatataccagtgatttttggggtaccattcagtttaaaccggatgaatatctgattcagaatggttttaccagtcaggttgcccgtaattttgtgaccaatcagagctttctgaattcactggttgattttaccccggccaatgcaggtaccaattatcgtgtggtggtggatccggatggtaatctgaccaatcagaatcttccgctgaaagtgcagattcagtatctggatggtaaatattatgacgcaaaactgaaaaacaacaacctggtgacctttagctataataattttgcagcactgccgagttgggtggttccgacccatcatcatcatcaccat

SEQ ID NO: 39 - protein, 916 aa, synthetic construct
  source 1..916: note = Recombinant Crm-EC amino acid sequence
MSFGFKQSDKSNDNTQLVNQARTLDANSVRLAGLGQNGSLFNTVLRDVDDNFITAANGTIIKLDSFTKPLYGLDLSDDFAGYKVKQIVSDYTTSRNRFDQRQTRAYYALLVNDEANVHLKRINTNSNRIGNRNNNSKFVIGGVDNPAHVIRFTDDGTKFNFTKQTQGEIVNDFILDAPILPKDLHPDWYNLYIQRKILPNDVNTAVVPWPVGRVSGTNADDGMFDFGNGQITNTDPIAQTKTTTDNQNPSTFNSGAMPGANNRYDSQLNVKHRIKTSFQLDEKFVYPEWTGSEENKNITRLATGSLPSNERYWILDIPGTPQVTLKEDSVNVFSRLYLNSVNSLSFIGDSIYIFGTSELPSLWYYSFPTRLSDLTALNQVKTDDIEASSTDNGTTTNGTTTTTDTSSGSTGAGTGNTTNTSQTVSNPTLNTYRSFGIDSKPTSANKIDETNWADPNVIEARIYAEYRLGIQNEIPITNAGNFIRNTIGGVGFTSTGSRVVLRASYNGDQRPTGNFQPFLYVFGYLGYQQTRTGTFWYGTYKLLNNSPYDVLDAARVGTETNQFRRTSLTYPVMGGYLTEEGARSFSNTPYIRAQGDTPESRSIFQSGYSDNTYEYIQSVLGFDGIRNNLNVGVKASSFLNSNRPNPNGLEMIAATTYLRSQIGLARTSGLPNQQPFGTTHQVISVSPGDQFSSIKNIRTIFPGNQLWYFLFTNENNKSSVYTLRLADSSNPDASSSFSPTSLIDVNEIGVILPLLDNSFYTVNAAGNVALFSSNPGSPGSYTAVNTFNQNLSDIAFEGSGAKYTSDFWGTIQFKPDEYLIQNGFTSQVARNFVTNQSFLNSLVDFTPANAGTNYRVVVDPDGNLTNQNLPLKVQIQYLDGKYYDAKLKNNNLVTFSYNNFAALPSWVVPTHHHHHH

SEQ ID NO: 40 - DNA, 2925 bp, synthetic construct
  source 1..2925: mol_type = other DNA; note = Recombinant Gap-EC nucleotide sequence
atgaagccggttaatcaggtgggcgaaatgaatagtcagggccagagcaatctgctggaaaaagcacgccgctggcgcaatagtaattttaccagtctgagtattgacggtaccaatccgggtgccctggttctgaccggcagtaaaagtattagccgtattgatctgtatggcaatgtgatttggacctttgatccgggcaataccaatgatctgaccggtaaagttggtttttatgatgccaataatcgtctgaccgcctttagtggtgacgtgccgtttaatgttagtgatctgagtagcaaaaccgttgtggaagcaacccaggatcaggaagatccgaatgtgttttatctgctgctgattccggatgcagccgtgcagcaggaacagaaaaccaaagatcaggtgtttgaaaattactttatgagtgatgccccggccaccggtgacaccagcgctgaaggtagcgccaccccggcaggcggtggttcaagcagcagcgccgccggtggcggtgcagttgcacctgcagccgccagtagtaccgcacgcctggtggaagaaggtaatagtgcaggtatgggtaccatgaccccgaccgccagcaccagcgaaaccgttattgattataatagcgatcagaataagatcccgaaaccgaaaaccctgctggatagcagcgaaagtagtgaaagcattaatggcggccgcacctatgcaaatattaatacccagaataacctgcagggcgttattgttaaagttaatgaaaatctgttcaacagcgaaaatccgtttgcagttgaaaatatggcattcattaagccgaaagatatggtggataattatccgagcacctggacccagggtagcgccaatggtaaaatgaccaatgtgctgcagttttataaacatgataacccgaatgccgttaataatcgcttttatcgtgcaaaatactacccgaaacgcctggaaacccagaccaccaccccgctgattgatagcagctttagcccgtatgaacatccggaatggtatgaagataatcagtttgttatgccgtggatgcagtatattaccaatctgggtggtctgtatgccaaagatggcatggtgtatctgtttggtggtaatggcacctgggtgaataatgaaagcgccctgagcattggcgtgtttcgcaccaaatttgaaaatcgtaccgccgaagccccgggtaataccaaaaccgttggttatccgtatggtattctgctgagcgccattagctttgatgcaacccgcaatggtctggcactggcaccggccagtctgggccaggatgtgggttatcattttgtgccgcgtctggccgtgggtggcgttagcagtccgcgtggtgcaaatggcaatatttttctgggtagtgcaattacctggggtaccaatggtggtaattttctggataccaaatggcatagtccggccgttattgaagatgccccgaccacctttgttaccgtgaatagcagtggcgcactgcagaatagcggtaatccgcagccgaccagcaccccgatgccgaatagtaatggtaatgaaagcattccgtatcgctggaccaatagctatgattataattcagtgcgctttgcagccctgattagcaaaccggcaggcggcaataccaaacaggttgaaagtctgtttaccaccgcactgaaactggataccctgaatagcctgccgaataagtttacccaggaaaataatattttcttcagctatgcaatgctggatggccgccagtggagcctgggtacccgcaaagatagtgcatggctgaccaccaataccattaataattttacctacaacacccagcagcagctggccagcaccgtggcaggtgaaaatgcaaatccgcgcaatattctgaatgccctgaccaccgcaaaaggttttgatcgtcgtgatattggtaatgttgtttatacctatagcaacaataccaataagttcacctattattaccaggttggtggtgccattaccacctggccggaagtgcaggtgaattataaaaccagcgcaaatattacctactataatctgacccgtaccgattttggtagcaccaccccggccacccaggatgcaaataccgttagtagtaaactgaatggcgcctatctgagtagtaccggtgaccagcagggctggtataatggtagcatctatgtgaaaaaagcaagttttaccccgagtagtcagggctatacctggcaggattttaaaggtctgaccaccaccgccagtaatgcagtgattagcaattggaccaaagccggctatagcattcgtccggatgatgataccgtttttaatgtgagcaaaattccgtttgaaaaggaaattaccgccgcagtgaatgtgcgcagtctggatagttattatgttcagctgaatggtgaaaccagcgttaataccgttgcccgtgttagtccggatagcagcgcactggcactgaatccgaatcgtattaccaatccgctgatgaatcgcgataatgtgattggccagggcgcattcattagtcgcaatgatattccgagtagctttttcgaaaataagattaatgacatcgtgaccaccgaagcagatggtaaagaagtgctggatagtaaatatattaacagtatctaccgctacaccccgccgcagaataatccggatattcgcctgcgtctgctggttattgatcgcagccgtgcaaccaatgattttattaagctgctgccgcaggtgctggttgatggtgaatatgttgcagttccgcaggccaatagtgtgtttgttagcgatcaggaattcactggctttgatgcactgccgggttatgtgctgccgcatcatcatcatcaccatcatcatcatcat

SEQ ID NO: 41 - protein, 975 aa, synthetic construct
  source 1..975: note = Recombinant GapEC amino acid sequence
MKPVNQVGEMNSQGQSNLLEKARRWRNSNFTSLSIDGTNPGALVLTGSKSISRIDLYGNVIWTFDPGNTNDLTGKVGFYDANNRLTAFSGDVPFNVSDLSSKTVVEATQDQEDPNVFYLLLIPDAAVQQEQKTKDQVFENYFMSDAPATGDTSAEGSATPAGGGSSSSAAGGGAVAPAAASSTARLVEEGNSAGMGTMTPTASTSETVIDYNSDQNKIPKPKTLLDSSESSESINGGRTYANINTQNNLQGVIVKVNENLFNSENPFAVENMAFIKPKDMVDNYPSTWTQGSANGKMTNVLQFYKHDNPNAVNNRFYRAKYYPKRLETQTTTPLIDSSFSPYEHPEWYEDNQFVMPWMQYITNLGGLYAKDGMVYLFGGNGTWVNNESALSIGVFRTKFENRTAEAPGNTKTVGYPYGILLSAISFDATRNGLALAPASLGQDVGYHFVPRLAVGGVSSPRGANGNIFLGSAITWGTNGGNFLDTKWHSPAVIEDAPTTFVTVNSSGALQNSGNPQPTSTPMPNSNGNESIPYRWTNSYDYNSVRFAALISKPAGGNTKQVESLFTTALKLDTLNSLPNKFTQENNIFFSYAMLDGRQWSLGTRKDSAWLTTNTINNFTYNTQQQLASTVAGENANPRNILNALTTAKGFDRRDIGNVVYTYSNNTNKFTYYYQVGGAITTWPEVQVNYKTSANITYYNLTRTDFGSTTPATQDANTVSSKLNGAYLSSTGDQQGWYNGSIYVKKASFTPSSQGYTWQDFKGLTTTASNAVISNWTKAGYSIRPDDDTVFNVSKIPFEKEITAAVNVRSLDSYYVQLNGETSVNTVARVSPDSSALALNPNRITNPLMNRDNVIGQGAFISRNDIPSSFFENKINDIVTTEADGKEVLDSKYINSIYRYTPPQNNPDIRLRLLVIDRSRATNDFIKLLPQVLVDGEYVAVPQANSVFVSDQEFTGFDALPGYVLPHHHHHHHHHH

SEQ ID NO: 42 - DNA, 2055 bp, Mycoplasma gallisepticum
  source 1..2055: mol_type = genomic DNA; note = VlhA 4.07.1 native nucleotide sequence
gtgaaaagaaaaaacattttaaagtttgttagtttattaggtattggttcgtttgtaatgttagcagctgctagctgtactcaagctagcactccaactccaactccaactccaactccaacacctaaccctgaaccaaagccaaatcctggtggtggtgacacgaataatcctcctagtggtggtatgaatggcgtagatactaatccaggtggtggacaaggcatgatgaatgctactaaccaggaattagtgaatgctaaaaaagctttaagtgatttaattggtggagaatctaagactgttgaattatatgcagactatgcaaaaattaaagctgacttgacttctgcgtacgcagttgctaaaacgacttcagatagttcaacatcaactttagatcaagttaaaacagcaacatcaacgttacaaactgctattaatactgctgctagtgacaaagaaaaatttgatcaacaaaatagccaattattaatggcatacaaagttttaaaagacactcttaataaaaaagaagctatagtaatgtcattaaatcaagaaaaatatagcgcaattttgagtgaaattaatgcagcttcttcaacagctgaagaaatcgttaaacaaacattgaatccagtgaatggtaatcttccagttgtagctacacttaatgctgaaaacaccaaaatcctagaagcaataaaagaagaaaaaattaattcagaaaaaagtaatgctgatctgtttgcaaattatcaattgtataaattggatagaactaaattaatgagtgaaggttctaataatacgaaacaaccaggaaattatagctttgtagcttatgctagtgacatagcaagtccaaactgaaattttgcacaaagaacagtttgaacagcagatagtagaacttgaaccagccctttacctaataattcacaaaactctgctcctttaacagatgtgtcatgaatctacactttaagcgggacaggagctaaatatacactaacatttgattattatggcccacaaactggttatttatactttccttataagttggctaaaaccagtgataaagtcgggcttcaatataaattaaatcaagcagattctgtagcgattcagtttagtgaagcggctgctactgcttcagctccagctgaaacagatggtagacaagaatcagctgaaactacaactgctaatgagaaagttaacccaatgccatcagttaatactattaatgttgctaaagtgactttatctaatttaaagtttggttctaacacaattgaatttagtgttccaatggatcaagacaatatgaataaagttgcgccaatgattggtaatatctatattacttcatctaatgatgaagcaaataaaaagcaaatttacgatagtatttttggaaacacttcatcacaaactgctagccaaacatctgttagtgttgatctattaaaaggatatagtcttgcaactagttgaagcacatatattcgtcaatttactggtttaacaggtaatggtgtacaaacctctgaccttgtttatttaattggtttgattggtggtagccagaatcgtacagttgcaagcggtccaatgaatattaaaaatattcctaatatgaatggcgctacaagaacattcacaatatatgcaaatgcaccagtaaacgggaactatcacataagtggtgcatatttacaaggagcgaatcaagcaagaagtctaaaattctcaaccggtacaagtggcagtaataatgaagttacagtcactggtttaaaacaaaaaaattgaaccacattaggtcactttgatactaagatgactagcactactgtttcaggaacaggtacaccaatgaaaagaactctaaccttaaatcagggtttaaacaagattgttgtatcaggagtaaacaatggcgatacaccatttataggtaatctaacatttactttagaaagcaatcaaacgattagtgaacaagttgagcctagttctgctactgcagaaaagaatatataa

SEQ ID NO: 43 - protein, 684 aa, Mycoplasma gallisepticum
  source 1..684: note = Native VlhA 4.07.1 amino acid sequence
MKRKNILKFVSLLGIGSFVMLAAASCTQASTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNSQNSAPLTDVSWIYTLSGTGAKYTLTFDYYGPQTGYLYFPYKLAKTSDKVGLQYKLNQADSVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLATSWSTYIRQFTGLTGNGVQTSDLVYLIGLIGGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNI

SEQ ID NO: 44 - DNA, 2094 bp, synthetic construct
  source 1..2094: mol_type = other DNA; note = Recombinant VlhA 4.07.1 nucleotide sequence
atgaaagcgaccaaactggtgctgggcgcggtgattctgggcagcaccctgctggcgggctgcagcagcaaccatggcattcgcggcagcattcgcaccacccaagcatcgaccccgaccccgaccccgaccccgaccccgacgccgaatccggaaccgaaacctaacccgggtggcggcgacaccaataatccgccgagcggcggcatgaacggcgtggacaccaacccgggcggcggccagggcatgatgaatgcgaccaaccaggagctggttaacgcaaaaaaagcgctgagcgatctgatcggcggcgaaagcaaaaccgtggaactgtatgccgattatgccaagattaaagcggatctgaccagcgcgtatgcggtggcgaaaaccaccagcgatagcagcacctccaccctggatcaggtgaaaaccgcgaccagcaccctgcagaccgccattaataccgccgccagcgataaagaaaaatttgaccagcagaatagccagctgctgatggcgtacaaagtactgaaagataccctgaataagaaagaagcgattgtgatgtcactgaaccaggaaaaatacagcgcgattctgagcgaaatcaacgccgcgagcagcaccgcggaagaaattgtgaaacagaccctgaacccggtgaatggtaatctgccggtggtggcaaccctgaatgcggaaaataccaaaatcctggaagcgattaaagaagaaaaaattaacagcgaaaaaagcaacgcagatctgtttgcgaattatcagctgtacaaactggatcgtaccaaactgatgagtgaaggcagcaacaataccaaacagccgggcaattatagctttgtggcatacgcgagcgacattgcgagcccgaactggaattttgcacaacgcaccgtgtggacggctgatagccgtacctggaccagcccgctgccgaacaacagccagaatagcgcgccgctgaccgatgtgagctggatttataccctgagcggcaccggcgcgaaatataccctgaccttcgattattatggcccgcagaccggctatctgtactttccgtacaaactggcgaaaaccagcgataaagtgggcctgcagtacaaactgaaccaggcggatagcgtggcgattcaattcagcgaagccgccgcgaccgcgagcgccccggcggaaaccgatggccgccaggaatcagcggaaaccaccaccgcgaacgaaaaagtgaacccgatgccgagcgtgaacacgatcaacgtcgcgaaagttaccctcagcaacctgaaattcggcagcaataccatcgaatttagcgtgccgatggatcaggataacatgaacaaagtggcgccgatgattggcaacatttatattaccagcagcaacgatgaagcgaacaaaaaacagatttatgatagcatttttggcaacaccagcagccagaccgcgagccagaccagcgtgagcgtggatctgctgaaaggctactccctggcgaccagctggtcaacatacatccgccagtttaccggcctgaccggcaatggcgtgcagaccagcgatctggtgtacctgattggcctgattggcggcagccagaatcgtaccgtggcaagcggcccgatgaatattaaaaacattccgaatatgaatggtgcgacccgcacctttaccatttacgcgaacgcgccggtgaacggcaactaccatatcagcggggcgtatctgcagggtgcgaaccaggcccgtagcctgaaatttagcaccggtacctcgggcagcaataacgaagttaccgttaccggcctgaaacagaaaaattggaccaccctgggccacttcgataccaaaatgaccagcaccaccgtgagcggtacgggcaccccgatgaaacgcaccctgaccctgaaccagggcctgaataaaattgtggtgagcggcgtgaacaatggcgataccccgtttattggcaacctgacctttaccctggaaagcaaccagaccattagcgaacaggtggaaccgagctcagcgaccgcggaaaagaacattgaaaacctgtattttcagagc

SEQ ID NO: 45 - protein, 698 aa, synthetic construct
  source 1..698: note = Recombinant VlhA 4.07.1 amino acid sequence
MKATKLVLGAVILGSTLLAGCSSNHGIRGSIRTTQASTPTPTPTPTPTPNPEPKPNPGGGDTNNPPSGGMNGVDTNPGGGQGMMNATNQELVNAKKALSDLIGGESKTVELYADYAKIKADLTSAYAVAKTTSDSSTSTLDQVKTATSTLQTAINTAASDKEKFDQQNSQLLMAYKVLKDTLNKKEAIVMSLNQEKYSAILSEINAASSTAEEIVKQTLNPVNGNLPVVATLNAENTKILEAIKEEKINSEKSNADLFANYQLYKLDRTKLMSEGSNNTKQPGNYSFVAYASDIASPNWNFAQRTVWTADSRTWTSPLPNNSQNSAPLTDVSWIYTLSGTGAKYTLTFDYYGPQTGYLYFPYKLAKTSDKVGLQYKLNQADSVAIQFSEAAATASAPAETDGRQESAETTTANEKVNPMPSVNTINVAKVTLSNLKFGSNTIEFSVPMDQDNMNKVAPMIGNIYITSSNDEANKKQIYDSIFGNTSSQTASQTSVSVDLLKGYSLATSWSTYIRQFTGLTGNGVQTSDLVYLIGLIGGSQNRTVASGPMNIKNIPNMNGATRTFTIYANAPVNGNYHISGAYLQGANQARSLKFSTGTSGSNNEVTVTGLKQKNWTTLGHFDTKMTSTTVSGTGTPMKRTLTLNQGLNKIVVSGVNNGDTPFIGNLTFTLESNQTISEQVEPSSATAEKNIENLYFQS

SEQ ID NO: 46 - DNA, 2004 bp, Mycoplasma gallisepticum
  source 1..2004: mol_type = genomic DNA; note = Native VlhA 4.07.6 nucleotide sequence
gtgaagagaaaaaacattttaaagtttgttagtttattaggtattggttcgtttgtgatgttggctgcagctagttgtacttcagcaactacaccaacacctaacccaacaccaaagccagatccaatgccaaatcctggtggtggtatgaatggtggaaatactaatcgagggaatggtggtggtatgatgggggataatcctaatcctgggaacactacaccagaacaacaattagcagctgctagaaaaacgctaacggatctacttggtacagaaaataccaacgttgccttttatgctgattatgccaagattcaaagcactttaaacgctgcttatatgacagctaaaactgcatcagaaaatacaagtgcatctttagaaaatctaagatctgcatcaactacactacaagcagctattgataaagctgctagtgataaacgtgcatttgatagtgctaatcaatctttagtaactgcatataataatttaaaaactacactgcaatttaaaacaactagtttagaagaattgtctgaaaataaatatagtggtattaaaaatcacttaagtaaactgtttgatacaggtagtgcaataacagctaaaacattagatcctactagtggtgaaagacctactttagagaaagtgaacgaagctaacaatagcattaaaatggctattagtcctgaatcgttgaagaaatgaaaagataatgcagacaaatttaatgagtttgagaaaaacccgttgtctaaagagaagttatctacaggaacagattcagctcataatcaagaacaaccagcaaactatagttttgtaggttatagcgttgatgtaactggaactactacagctcctgcttcagaaaaagctcttcttaactggaactatgctcagagaacgatttttacaagtggtgatcaaccaactaagattgatactcctgcagcttcagacgaagctccagctcaacctttatcaaatgtttcttgaatttatagtttagcaggtaatggagctaaatatacgctagatttcacctactatggaccatcaacaggttatttatatttcccttataagttagttaaaaatagtgatagtgttggtttacaatataagttaaataatagtcctactgaaacagcaatcgcttttggtaatatgcaaaatgccaacggtccaactgcaacagtggatagtattaatatagctaaagttactttatctaatttaaattttggagcaaacaaaattgaattcagtgttccttcagaaaaagtagctccgatgattggtaatatgtatcttacttcatcagctgatgaagctaataaacaaaaaatcgaaaatagtatttttggtaatagtgttacaactgaaaataatagaacaataatttcagttgatgctttaagtggttatagtttagcttctgattgatcaacatttattggaaggtatgcagaccaacagttaaccttagatggaaaaatgatgcgtgatcaaaaatattatctaatcggttatgttggtggtaatactggcgctcgtaatgttactggaataacagaaaaaacaaacgagcaaagatctccagtagttagtactccaaaaacaagaacttatacattctatgtaaatgctccaaaagcaggtgcttattatattaaaggagtgttcgcttcaaaggttcgtagagatcttaaattcagtaccggagatatgtcttctaataatgtaactatacaacaattgtctacaaataatttaactacattaagaacctttgacacctctgcaacaacaggagctacccgagttacaactgtttctgatagaaagacattaactctagtagagggattaaataagatagtagttagtggagctactgcagataatggtaatgctccaaattttggatatttagaatttattcttaatgaaacccaacctgaaactactaatgtttcaaatccatcttag

SEQ ID NO: 47 - protein, 667 aa, Mycoplasma gallisepticum
  source 1..667: note = Native VlhA 4.07.6 amino acid sequence
MKRKNILKFVSLLGIGSFVMLAAASCTSATTPTPNPTPKPDPMPNPGGGMNGGNTNRGNGGGMMGDNPNPGNTTPEQQLAAARKTLTDLLGTENTNVAFYADYAKIQSTLNAAYMTAKTASENTSASLENLRSASTTLQAAIDKAASDKRAFDSANQSLVTAYNNLKTTLQFKTTSLEELSENKYSGIKNHLSKLFDTGSAITAKTLDPTSGERPTLEKVNEANNSIKMAISPESLKKWKDNADKFNEFEKNPLSKEKLSTGTDSAHNQEQPANYSFVGYSVDVTGTTTAPASEKALLNWNYAQRTIFTSGDQPTKIDTPAASDEAPAQPLSNVSWIYSLAGNGAKYTLDFTYYGPSTGYLYFPYKLVKNSDSVGLQYKLNNSPTETAIAFGNMQNANGPTATVDSINIAKVTLSNLNFGANKIEFSVPSEKVAPMIGNMYLTSSADEANKQKIENSIFGNSVTTENNRTIISVDALSGYSLASDWSTFIGRYADQQLTLDGKMMRDQKYYLIGYVGGNTGARNVTGITEKTNEQRSPVVSTPKTRTYTFYVNAPKAGAYYIKGVFASKVRRDLKFSTGDMSSNNVTIQQLSTNNLTTLRTFDTSATTGATRVTTVSDRKTLTLVEGLNKIVVSGATADNGNAPNFGYLEFILNETQPETTNVSNPS

SEQ ID NO: 48 - DNA, 2043 bp, synthetic construct
  source 1..2043: mol_type = other DNA; note = Recombinant VlhA 4.07.6 nucleotide sequence
atgaaagcgaccaaactggtgctgggcgcggtgattctgggcagcaccctgctggcgggctgcagcagcaaccatggcattcgcggcagcattcgcaccaccagcgcgaccaccccgaccccgaatccgaccccgaaaccggatccgatgccgaacccgggcggcggcatgaatggtggcaataccaaccgcggcaatggcggcggtatgatgggcgataatccgaatccgggcaacaccaccccggaacagcagctggccgccgcgcgcaaaaccctgaccgatctgctgggtaccgaaaacaccaatgttgcattctatgcagattacgcgaaaattcagtcaaccctgaatgcggcctacatgaccgcgaaaaccgccagcgaaaacaccagcgcgagcctcgaaaacctgcgtagcgccagcaccacactgcaggcggcgattgataaagcggcgagcgataaacgcgcgtttgattctgcaaatcagagcctggtgactgcgtacaacaatctgaaaaccaccctgcagtttaaaacgaccagcctggaagaattaagcgaaaacaaatatagcggaattaaaaaccacctgagcaaactgtttgataccggtagcgccattaccgccaaaaccctggatccgaccagcggcgaacgcccgaccctggaaaaagttaacgaagcgaataacagcattaaaatggcgatttcgccggaaagcctgaaaaaatggaaagataacgccgataaatttaatgaatttgaaaaaaacccgctgagcaaggaaaaactgtcgaccggcaccgacagcgcgcacaaccaggaacagccggcgaactatagctttgtgggttacagcgtggatgtgaccggcaccaccaccgccccggcgagcgaaaaagcgctgctgaattggaactatgcgcagcgcacgatttttaccagcggcgatcagccgaccaaaattgacaccccggcggcctcggatgaagccccggcgcagccgctgagcaacgtgagctggatttatagcctggcgggcaacggcgcgaaatacaccctggattttacctactacggcccgtcaaccggctatctgtattttccgtataaactggtgaaaaacagcgattcagtgggcctgcagtacaaactgaataacagcccgaccgaaaccgccattgcgttcggcaacatgcagaacgccaacggcccgaccgccaccgtggatagcattaacattgccaaagttacgctgagcaacctgaattttggcgcgaataaaattgaattctcggtgccgagcgaaaaagtggcgccgatgatcggcaatatgtatctgaccagcagcgcggatgaagcaaacaaacagaaaattgaaaactcaatttttggcaacagcgtgaccaccgaaaataatcgcaccatcatttccgtcgatgcgctgagcggctacagcctggcgagcgattggagcaccttcattggccgttatgcggatcagcagctgaccctggatggcaaaatgatgcgcgatcagaaatattatctgattggctatgtgggcggcaataccggcgcgcgcaacgtgaccggcattaccgaaaaaaccaatgaacagcgcagcccggttgttagcaccccgaaaacccgcacgtataccttttatgtgaatgcgccgaaagcgggtgcgtactatattaaaggtgtgttcgcctcgaaagtgcgccgtgatctgaaattcagtaccggcgatatgagctcgaacaacgtgaccattcagcagctgagcaccaacaacctgaccaccctgcgcaccttcgataccagcgcgaccaccggcgcgacccgtgttaccaccgtgagcgaccgtaaaaccctgaccctggtcgaaggcctgaacaaaattgtggtgagcggcgcgaccgcggataacggcaatgccccgaactttggctacctggaattcattctgaacgaaacccagccggaaaccaccaatgtgagcaatccgagcgaaaacctgtattttcagagc

SEQ ID NO: 49 - protein, 681 aa, synthetic construct
  source 1..681: note = Recombinant VlhA 4.07.6 amino acid sequence
MKATKLVLGAVILGSTLLAGCSSNHGIRGSIRTTSATTPTPNPTPKPDPMPNPGGGMNGGNTNRGNGGGMMGDNPNPGNTTPEQQLAAARKTLTDLLGTENTNVAFYADYAKIQSTLNAAYMTAKTASENTSASLENLRSASTTLQAAIDKAASDKRAFDSANQSLVTAYNNLKTTLQFKTTSLEELSENKYSGIKNHLSKLFDTGSAITAKTLDPTSGERPTLEKVNEANNSIKMAISPESLKKWKDNADKFNEFEKNPLSKEKLSTGTDSAHNQEQPANYSFVGYSVDVTGTTTAPASEKALLNWNYAQRTIFTSGDQPTKIDTPAASDEAPAQPLSNVSWIYSLAGNGAKYTLDFTYYGPSTGYLYFPYKLVKNSDSVGLQYKLNNSPTETAIAFGNMQNANGPTATVDSINIAKVTLSNLNFGANKIEFSVPSEKVAPMIGNMYLTSSADEANKQKIENSIFGNSVTTENNRTIISVDALSGYSLASDWSTFIGRYADQQLTLDGKMMRDQKYYLIGYVGGNTGARNVTGITEKTNEQRSPVVSTPKTRTYTFYVNAPKAGAYYIKGVFASKVRRDLKFSTGDMSSNNVTIQQLSTNNLTTLRTFDTSATTGATRVTTVSDRKTLTLVEGLNKIVVSGATADNGNAPNFGYLEFILNETQPETTNVSNPSENLYFQS

SEQ ID NO: 50 - protein, 10 aa, Synthetic construct
  source 1..10: note = 10-his tag
HHHHHHHHHH